(19) United States
(12) Patent Application Publication
(10) Pub. No.: US 2017/0016001 A1
(43) Pub. Date: Jan. 19, 2017

(54) ASYMMETRIC INTERFERING RNA COMPOSITIONS THAT SILENCE K-RAS AND METHODS OF USES THEREOF

(71) Applicant: BOSTON BIOMEDICAL, INC., Cambridge, MA (US)

(72) Inventor: Chiang Jia LI, Cambridge, MA (US)

(73) Assignee: Boston Biomedical, Inc., Cambridge, MA (US)

(21) Appl. No.: 15/125,655

(22) PCT Filed: Mar. 16, 2015
(86) PCT No.: PCT/US15/20776

§ 371 Date: Sep. 13, 2016

Related U.S. Application Data
(60) Provisional application No. 61/953,590, filed on Mar. 14, 2014.
(60) Provisional application No. 62/121,721, filed on Feb. 27, 2015.

(51) Int. Cl. C12N 15/113; C12Q 1/68; G01N 33/574

(52) U.S. Cl. CPC C12N 15/1135; G01N 33/5748; C12Q 1/6886; C12N 2310/14; G01N 2333/914; C12Q 2600/158

(57) ABSTRACT

The invention provides novel compositions for use in silencing K-Ras gene expression. More particularly, the invention provides novel asymmetrical interfering RNA molecules as inhibitors of K-Ras expression, and to pharmaceutical compositions and uses thereof in the treatment of cancer or a related disorder in a mammal.

### FIELD OF THE INVENTION

The invention generally relates to compositions for use in silencing K-Ras gene expression. More particularly, the invention relates to novel asymmetrical interfering RNA molecules as inhibitors of K-Ras expression, and to pharmaceutical compositions and uses thereof in the treatment of cancer or a related disorder in a mammal.

### BACKGROUND OF THE INVENTION

Gene silencing through RNAi (RNA-interference) by use of small or short interfering RNA (siRNA) has emerged as a therapeutic tool. However, other than the prominent delivery issue, the development of RNAi-based drugs faces challenges of limited efficacy of siRNA, non-specific effects of siRNA such as interferon-like responses and sense-strand mediated off-target gene silencing, and the prohibitive or high cost associated with siRNA synthesis. The gene silencing efficacy by siRNA is limited to about 50% or less for majority of genes in mammalian cells. The manufacture of these molecules is expensive (much more expensive than manufacturing anti sense deoxynucleotides), inefficient, and requires chemical modification. Finally, the observation that the extracellular administration of synthetic siRNAs can trigger interferon-like responses has added a significant barrier for RNAi-based research and RNAi-based therapeutic development.

The protein K-Ras is a molecular switch that under normal conditions regulates cell growth and cell division. Mutations in this protein lead to the formation of tumors through continuous cell growth. About 30% of human cancers have a mutated Ras protein that is constitutively bound to GTP due to decreased GTPase activity and insensitivity to GAP action. Ras is also an important factor in many cancers in which it is not mutated but rather functionally activated through inappropriate activity of other signal transduction elements. Mutated K-Ras proteins are found in a large proportion of all tumour cells. K-Ras protein occupies a central position of interest. The identification of oncogenically mutated K-Ras in many human cancers led to major efforts to target this constitutively activated protein as a rational and selective treatment. Despite decades of active agent research, significant challenges still remain to develop therapeutic inhibitors of K-Ras.

Hypermalignant cancer cells that are highly tumorigenic and metastatic have been isolated from cancer patients with a variety of tumor types and found to have high stemness properties, termed cancer stem cells (CSCs). These stemness-high cancer cells are hypothesized to be fundamentally responsible for cancer metastasis and relapse. A number of stemness genes, such as β-catenin, Nanog, Sox2, Oct3/4 have been implicated in cancer cell stemness. However, the role of oncogenes, such as K-Ras, in cancer cell stemness is not clear.

Accordingly, there exists a need to develop novel compositions and methods for selectively silencing K-Ras gene express or K-Ras activity in a subject diagnosed with cancer, with better efficacy and potency, rapid onset of action, better durability, and fewer adverse side effects.

### SUMMARY OF THE INVENTION

To elucidate the role of K-Ras in the maintenance of cancer cell stemness, the present inventors employed asymmetric silencing RNA technology (aiRNA) which is able to silence target genes with high potency and precision. Moreover, aiRNA technology can be readily applied to CSCs. The present inventors made a surprising discovery that CSCs are not only addicted to activating mutations of K-Ras, or activation of the downstream regulators of the Ras pathway, but also that CSCs with amplified mutant K-Ras become highly sensitive to K-Ras silencing. Furthermore, the present inventors made a surprising discovery that the DNA copy numbers of the mutant K-Ras directly predicts sensitivity of cancer stem cells to K-Ras silencing, which suggests that amplified mutated K-Ras is required to the maintenance of the malignancy and cancer cell stemness, which may have significant implication for understanding the connection between oncogene and cancer cell stemness and for developing cancer stem cell inhibitors.

The present invention provides compositions and methods that use a class of small duplex RNA that can induce potent gene silencing in mammalian cells, which is termed herein asymmetrical interfering RNAs (aiRNA). aiRNA is described, for example, in PCT Publication No. WO 2009/029688, the contents of which are hereby incorporated by reference in their entirety. This class of RNAi-inducers is identified by the length asymmetry of the two RNA strands. This structural design is not only functionally potent in effecting gene silencing, but offers several advantages over the current state-of-art siRNAs. Among the advantages, aiRNA can have RNA duplex structure of much shorter length than the other siRNA, which should reduce the cost of synthesis and abrogate/reduce the length-dependent triggering of nonspecific interferon-like responses. In addition, the asymmetry of the aiRNA structure abrogates and/or otherwise reduces the sense-strand mediated off-target effects. Furthermore, aiRNA is more efficacious, potent, rapid-onset, and durable than siRNA in inducing gene silencing. AiRNA can be used in all areas that other siRNA or shRNA are being applied or contemplated to be used, including biology research, R&D research in biotechnology and pharmaceutical industry, and RNAi-based therapies.

The duplex RNA molecule comprises a first strand with a length from 18-23 nucleotides and a second strand with a length from 12-17 nucleotides, wherein the second strand is substantially complementary to the first strand, and forms a double-stranded region with the first strand, wherein the first strand has a 3′-overhang from 1-9 nucleotides, and a 5′-overhang from 0-8 nucleotides, wherein said duplex RNA molecule is capable of effecting selective K-Ras gene silencing in a eukaryotic cell. In some embodiments, the first strand comprises a sequence being substantially complementary to a target K-Ras mRNA sequence. In a further embodiment, the first strand comprises a sequence being at least 70 percent complementary to a target K-Ras mRNA sequence. In another embodiment, the eukaryotic cell is a mammalian cell or an avian cell.

In some embodiments, the target K-Ras mRNA sequence is a human K-Ras target sequence. In some embodiments, the target K-Ras mRNA sequence is a human K-Ras target sequence selected from at least a portion of the sequence shown in GenBank Accession No. NM_004985 shown below as SEQ ID NO: 1:

| (SEQ ID NO: 1) | |
|---|---|
| 1 | tcctaggcgg cggccgcggc ggcggaggca gcagcggcgg cggcagtggc ggcggcgaag |
| 61 | gtggcggcgg ctcggccagt actcccggcc cccgccattt cggactggga gcgagcgcgg |
| 121 | cgcaggcact gaaggcggcg gcggggccag aggctcagcg gctcccaggt gcgggagaga |
| 181 | ggcctgctga aaatgactga atataaactt gtggtagttg gagctggtgg cgtaggcaag |
| 241 | agtgccttga cgatacagct aattcagaat cattttgtgg acgaatatga tccaacaata |
| 301 | gaggattcct acaggaagca agtagtaatt gatggagaaa cctgtctctt ggatattctc |
| 361 | gacacagcag gtcaagagga gtacagtgca atgagggacc agtacatgag gactggggag |
| 421 | ggctttcttt gtgtatttgc cataaataat actaaatcat ttgaagatat tcaccattat |
| 481 | agagaacaaa ttaaaagagt taaggactct gaagatgtac ctatggtcct agtaggaaat |
| 541 | aaatgtgatt tgccttctag aacagtagac acaaaacagg ctcaggactt agcaagaagt |
| 601 | tatggaattc cttttattga aacatcagca aagacaagac agggtgttga tgatgccttc |
| 661 | tatacattag ttcgagaaat tcgaaaacat aaagaaaaga tgagcaaaga tggtaaaaag |
| 721 | aagaaaaaga agtcaaagac aaagtgtgta attatgtaaa tacaatttgt acttttttct |
| 781 | taaggcatac tagtacaagt ggtaattttt gtacattaca ctaaattatt agcatttgtt |
| 841 | ttagcattac ctaatttttt tcctgctcca tgcagactgt tagcttttac cttaaatgct |
| 901 | tattttaaaa tgacagtgga agtttttttt tcctctaagt gccagtattc ccagagtttt |
| 961 | ggtttttgaa ctagcaatgc ctgtgaaaaa gaaactgaat acctaagatt tctgtcttgg |
| 1021 | ggtttttggt gcatgcagtt gattacttct tatttttctt accaattgtg aatgttggtg |
| 1081 | tgaaacaaat taatgaagct tttgaatcat ccctattctg tgttttatct agtcacataa |
| 1141 | atggattaat tactaatttc agttgagacc ttctaattgg tttttactga aacattgagg |
| 1201 | gaacacaaat ttatgggctt cctgatgatg attcttctag gcatcatgtc ctatagtttg |
| 1261 | tcatccctga tgaatgtaaa gttacactgt tcacaaaggt tttgtctcct ttccactgct |
| 1321 | attagtcatg gtcactctcc ccaaaatatt atattttttc tataaaaaga aaaaaatgga |
| 1381 | aaaaaattac aaggcaatgg aaactattat aaggccattt ccttttcaca ttagataaat |
| 1441 | tactataaag actcctaata gcttttcctg ttaaggcaga cccagtatga aatggggatt |
| 1501 | attatagcaa ccattttggg gctatattta catgctacta aatttttata ataattgaaa |
| 1561 | agattttaac aagtataaaa aattctcata ggaattaaat gtagtctccc tgtgtcagac |
| 1621 | tgctctttca tagtataact ttaaatcttt tcttcaactt gagtctttga agatagtttt |
| 1681 | aattctgctt gtgacattaa aagattattt gggccagtta tagcttatta ggtgttgaag |
| 1741 | agaccaaggt tgcaaggcca ggccctgtgt gaacctttga gctttcatag agagtttcac |
| 1801 | agcatggact gtgtccccac ggtcatccag tgttgtcatg cattggttag tcaaaatggg |
| 1861 | gagggactag ggcagtttgg atagctcaac aagatacaat ctcactctgt ggtggtcctg |
| 1921 | ctgacaaatc aagagcattg cttttgtttc ttaagaaaac aaactctttt ttaaaaatta |
| 1981 | cttttaaata ttaactcaaa agttgagatt ttggggtggt ggtgtgccaa gacattaatt |
| 2041 | ttttttttaa acaatgaagt gaaaaagttt tacaatctct aggtttggct agttctctta |
| 2101 | acactggtta aattaacatt gcataaacac ttttcaagtc tgatccatat ttaataatgc |
| 2161 | tttaaaataa aaataaaaac aatccttttg ataaatttaa aatgttactt attttaaaat |
| 2221 | aaatgaagtg agatggcatg gtgaggtgaa agtatcactg gactaggaag aaggtgactt |
| 2281 | aggttctaga taggtgtctt ttaggactct gattttgagg acatcactta ctatccattt |
| 2341 | cttcatgtta aaagaagtca tctcaaactc ttagtttttt ttttttacaa ctatgtaatt |
| 2401 | tatattccat ttacataagg atacacttat ttgtcaagct cagcacaatc tgtaaatttt |
| 2461 | taacctatgt tacaccatct tcagtgccag tcttgggcaa aattgtgcaa gaggtgaagt |
| 2521 | ttatatttga atatccattc tcgttttagg actcttcttc catattagtg tcatcttgcc |
| 2581 | tccctacctt ccacatgccc catgacttga tgcagtttta atacttgtaa ttcccctaac |
| 2641 | cataagattt actgctgctg tggatatctc catgaagttt tcccactgag tcacatcaga |
| 2701 | aatgccctac atcttatttc ctcagggctc aagagaatct gacagatacc ataaagggat |
| 2761 | ttgacctaat cactaatttt caggtggtgg ctgatgcttt gaacatctct ttgctgccca |
| 2821 | atccattagc gacagtagga tttttcaaac ctggtatgaa tagacagaac cctatccagt |
| 2881 | ggaaggagaa tttaataaag atagtgctga aagaattcct taggtaatct ataactagga |
| 2941 | ctactcctgg taacagtaat acattccatt gttttagtaa ccagaaatct tcatgcaatg |
| 3001 | aaaaatactt taattcatga agcttacttt ttttttttgg tgtcagagtc tcgctcttgt |
| 3061 | cacccaggct ggaatgcagt ggcgccatct cagctcactg caacctccat ctcccaggtt |
| 3121 | caagcgattc tcgtgcctcg gcctcctgag tagctgggat tacaggcgtg tgccactaca |
| 3181 | ctcaactaat ttttgtattt ttaggagaga cggggtttca ccctgttggc caggctggtc |
| 3241 | tcgaactcct gacctcaagt gattcaccca ccttggcctc ataaacctgt tttgcagaac |
| 3301 | tcatttattc agcaaatatt tattgagtgc ctaccagatg ccagtcaccg cacaaggcac |
| 3361 | tgggtatatg gtatccccaa acaagagaca taatcccggt ccttaggtag tgctagtgtg |
| 3421 | gtctgtaata tcttactaag gcctttggta tacgacccag agataacacg atgcgtattt |
| 3481 | tagttttgca aagaaggggt ttggtctctg tgccagctct ataattgttt tgctacgatt |
| 3541 | ccactgaaac tcttcgatca agctacttta tgtaaatcac ttcattgttt taaaggaata |
| 3601 | aacttgatta tattgttttt ttatttggca taactgtgat tcttttagga caattactgt |
| 3661 | acacattaag gtgtatgtca gatattcata ttgacccaaa tgtgtaatat tccagttttc |
| 3721 | tctgcataag taattaaaat atacttaaaa attaatagtt ttatctgggt acaaataaac |
| 3781 | aggtgcctga actagttcac agacaaggaa acttctatgt aaaaatcact atgatttctg |
| 3841 | aattgctatg tgaaactaca gatctttgga acactgttta ggtagggtgt taagacttac |
| 3901 | acagtacctc gtttctacac agagaaagaa atggccatac ttcaggaact gcagtgctta |
| 3961 | tgaggggata tttaggcctc ttgaattttt gatgtagatg ggcatttttt taaggtagtg |
| 4021 | gttaattacc tttatgtgaa ctttgaatgg tttaacaaaa gatttgtttt tgtagagatt |
| 4081 | ttaaaggggg agaattctag aaataaatgt tacctaatta ttacagcctt aaagacaaaa |
| 4141 | atccttgttg aagttttttt aaaaaaagct aaattacata gacttaggca ttaacatgtt |
| 4201 | tgtggaagaa tatagcagac gtatattgta tcatttgagt gaatgttccc aagtaggcat |
| 4261 | tctaggctct atttaactga gtcacactgc ataggaattt agaacctaac ttttataggt |
| 4321 | tatcaaaact gttgtcacca ttgcacaatt ttgtcctaat atatacatag aaactttgtg |
| 4381 | gggcatgtta agttacagtt tgcacaagtt catctcattt gtattccatt gatttttttt |
| 4441 | ttcttctaaa cattttttct tcaaacagta tataactttt tttaggggat ttttttttag |
| 4501 | acagcaaaaa ctatctgaag atttccattt gtcaaaaagt aatgatttct tgataattgt |
| 4561 | gtagtaatgt tttttagaac ccagcagtta ccttaaagct gaatttatat ttagtaactt |
| 4621 | ctgtgttaat actggatagc atgaattctg cattgagaaa ctgaatagct gtcataaaat |
| 4681 | gaaactttct ttctaaagaa agatactcac atgagttctt gaagaatagt cataactaga |
| 4741 | ttaagatctg tgttttagtt taatagtttg aagtgcctgt ttgggataat gataggtaat |
| 4801 | ttagatgaat ttaggggaaa aaaaagttat ctgcagatat gttgagggcc catctctccc |
| 4861 | cccacacccc cacagagcta actgggttac agtgttttat ccgaaagttt ccaattccac |
| 4921 | tgtcttgtgt tttcatgttg aaaatacttt tgcatttttc ctttgagtgc caatttctta |
| 4981 | ctagtactat ttcttaatgt aacatgttta cctggaatgt attttaacta tttttgtata |
| 5041 | gtgtaaactg aaacatgcac attttgtaca ttgtgctttc ttttgtggga catatgcagt |
| 5101 | gtgatccagt tgttttccat catttggttg cgctgaccta ggaatgttgg tcatatcaaa |
| 5161 | cattaaaaat gaccactctt ttaattgaaa ttaactttta aatgtttata ggagtatgtg |
| 5221 | ctgtgaagtg atctaaaatt tgtaatattt ttgtcatgaa ctgtactact cctaattatt |
| 5281 | gtaatgtaat aaaaatagtt acagtgacta tgagtgtgta tttattcatg aaatttgaac |
| 5341 | tgtttgcccc gaaatggata tggaatactt tataagccat agacactata gtataccagt |
| 5401 | gaatctttta tgcagcttgt tagaagtatc ctttatttct aaaaggtgct gtggatatta |
| 5461 | tgtaaaggcg tgtttgctta aacttaaaac catatttaga agtagatgca aaacaaatct |
| 5521 | gcctttatga caaaaaaata ggataacatt atttatttat ttccttttat caaagaaggt |
| 5581 | aattgataca caacaggtga cttggtttta ggcccaaagg tagcagcagc aacattaata |
| 5641 | atggaaataa ttgaatagtt agttatgtat gttaatgcca gtcaccagca ggctatttca |
| 5701 | aggtcagaag taatgactcc atacatatta tttatttcta taactacatt taaatcatta |
| 5761 | ccagg |

In some embodiments, the target K-Ras mRNA sequence is a target sequence shown in Table 1 below.

TABLE 1
Target K-Ras Sequences

| Target | Position in | | Targeted by |
|---|---|---|---|
| SEQ ID | NM_004985 | K-Ras Target | aiRNA ID |
| NO: | Sequence | Sequence | NO: |
| 2 | 1701 | GGCCAGTTATAGCTTATTA | 1 |
| 3 | 514 | GGTCCTAGTAGGAAATAAA | 2 |
| 4 | 1464 | GGCAGACCCAGTATGAAAT | 3 |
| 5 | 2010 | GGTGTGCCAAGACATTAAT | 4 |
| 6 | 2538 | GGACTCTTCTTCCATATTA | 5 |
| 7 | 1382 | GGCAATGGAAACTATTATA | 6 |
| 8 | 1024 | GCAGTTGATTACTTCTTAT | 7 |
| 9 | 574 | GGACTTAGCAAGAAGTTAT | 8 |
| 10 | 2427 | GCTCAGCACAATCTGTAAA | 9 |
| 11 | 1295 | CTCCTTTCCACTGCTATTA | 10 |
| 12 | 1063 | GTTGGTGTGAAACAAATTA | 11 |
| 13 | 240 | CGAUACAGCUAAUUCAGAA | 12 |
| 14 | 245 | CAGCUAAUUCAGAAUCAUU | 13 |
| 15 | 247 | GCUAAUUCAGAAUCAUUUU | 14 |
| 16 | 271 | CGAAUAUGAUCCAACAAUA | 15 |
| 17 | 2935 | CCTGGTAACAGTAATACAT | 16 |
| 18 | 569 | GCTCAGGACTTAGCAAGAA | 17 |
| 19 | 3495 | CTCTGTGCCAGCTCTATAA | 18 |
| 20 | 1508 | GGGCTATATTTACATGCTA | 19 |
| 21 | 330 | CCTGTCTCTTGGATATTCT | 20 |
| 22 | 406 | GGAGGGCTTTCTTTGTGTA | 21 |
| 23 | 2649 | GTGGATATCTCCATGAAGT | 22 |
| 24 | 461 | CACCATTATAGAGAACAAA | 23 |
| 25 | 3409 | GGTCTGTAATATCTTACTA | 24 |
| 26 | 234 | CCTTGACGATACAGCTAAT | 25 |
| 27 | 2779 | GCTGATGCTTTGAACATCT | 26 |
| 28 | 1251 | CATCCCTGATGAATGTAAA | 27 |
| 29 | 420 | GUGUAUUUGCCAUAAAUAA | 28 |
| 30 | 430 | CAUAAAUAAUACUAAAUCA | 29 |
| 31 | 441 | CUAAAUCAUUUGAAGAUAU | 30 |
| 32 | 452 | GAAGAUAUUCACCAUUAUA | 31 |
| 33 | 4055 | TGGTTTAACAAAAGATTTG | W32 |
| 34 | 4359 | TGTCCTAATATATACATAG | W33 |
| 35 | 991 | TGAAAAAGAAACTGAATAC | W34 |
| 36 | 2428 | CTCAGCACAATCTGTAAAT | 35 |
| 37 | 1611 | GCTCTTTCATAGTATAACT | 36 |
| 38 | 3399 | GTGCTAGTGTGGTCTGTAA | 37 |
| 39 | 3402 | CTAGTGTGGTCTGTAATAT | 38 |
| 40 | 4204 | GCAGACGTATATTGTATCA | 39 |
| 41 | 4234 | GTTCCCAAGTAGGCATTCT | 40 |
| 42 | 268 | GGACGAATATGATCCAACA | 41 |
| 43 | 304 | GAAGCAAGTAGTAATTGAT | 42 |
| 44 | 1206 | GCTTCCTGATGATGATTCT | 43 |
| 45 | 3237 | CCTGACCTCAAGTGATTCA | 44 |
| 46 | 2567 | GCCTCCCTACCTTCCACAT | W45 |
| 47 | 1403 | GCCATTTCCTTTTCACATT | W46 |
| 48 | 4207 | GACGTATATTGTATCATTT | W47 |
| 49 | 1402 | GGCCATTTCCTTTTCACAT | W48 |
| 50 | 4075 | GGGGGAGAATTCTAGAAAT | W49 |
| 51 | 4234 | GTTCCCAAGTAGGCATTCT | 50 |
| 52 | 268 | GGACGAATATGATCCAACA | 51 |
| 53 | 304 | GAAGCAAGTAGTAATTGAT | 52 |
| 54 | 1206 | GCTTCCTGATGATGATTCT | 53 |
| 55 | 5247 | GAACTGTACTACTCCTAAT | 54 |
| 56 | 3237 | CCTGACCTCAAGTGATTCA | 55 |
| 57 | 3386 | GTCCTTAGGTAGTGCTAGT | 56 |
| 58 | 1601 | GTGTCAGACTGCTCTTTCA | 57 |
| 59 | 1607 | GACTGCTCTTTCATAGTAT | 58 |
| 60 | 1255 | CCTGATGAATGTAAAGTTA | 59 |
| 61 | 2124 | CAAGTCTGATCCATATTTA | 60 |
| 62 | 688 | GATGAGCAAAGATGGTAAA | 61 |
| 63 | 2497 | CAAGAGGTGAAGTTTATAT | 62 |
| 64 | 3870 | GGTAGGGTGTTAAGACTTA | 63 |
| 65 | 1226 | CTAGGCATCATGTCCTATA | 64 |
| 66 | 4226 | GAGTGAATGTTCCCAAGTA | 65 |
| 67 | 517 | CCTAGTAGGAAATAAATGT | 66 |
| 68 | 3774 | GCCTGAACTAGTTCACAGA | 67 |
| 69 | 2970 | CCAGAAATCTTCATGCAAT | 68 |
| 70 | 2646 | GCTGTGGATATCTCCATGA | 69 |
| 71 | 303 | GGAAGCAAGTAGTAATTGA | 70 |
| 72 | 4203 | CAGACGTATATTGTATCAT | 71 |
| 73 | 233 | GCCTTGACGATACAGCTAA | 72 |
| 74 | 2259 | GAAGGTGACTTAGGTTCTA | 73 |
| 75 | 2076 | GGCTAGTTCTCTTAACACT | 74 |
| 76 | 3660 | GTGTATGTCAGATATTCAT | 75 |
| 77 | 1760 | GAACCTTTGAGCTTTCATA | 76 |
| 78 | 3789 | CAGACAAGGAAACTTCTAT | 77 |
| 79 | 3541 | CTTCGATCAAGCTACTTTA | 78 |
| 80 | 4954 | GAGTGCCAATTTCTTACTA | 79 |
| 81 | 1909 | GCTGACAAATCAAGAGCAT | 80 |
| 82 | 2346 | GTCATCTCAAACTCTTAGT | 81 |
| 83 | 638 | GATGATGCCTTCTATACAT | 82 |
| 84 | 2840 | CTGGTATGAATAGACAGAA | 83 |
| 85 | 2673 | CACTGAGTCACATCAGAAA | 84 |
| 86 | 4320 | GTTGTCACCATTGCACAAT | 85 |
| 87 | 2422 | GTCAAGCTCAGCACAATCT | 86 |
| 88 | 1484 | GGGATTATTATAGCAACCA | 87 |
| 89 | 2252 | CTAGGAAGAAGGTGACTTA | 88 |
| 90 | 493 | GGACTCTGAAGATGTACCT | 89 |
| 91 | 3135 | CTGAGTAGCTGGGATTACA | 90 |
| 92 | 4921 | CATGAGTTCTTGAAGAATA | 91 |
| 93 | 266 | GTGGACGAATATGATCCAA | 92 |
| 94 | 2647 | CTGTGGATATCTCCATGAA | 93 |
| 95 | 3791 | GACAAGGAAACTTCTATGT | 94 |
| 96 | 4197 | GAATATAGCAGACGTATAT | 95 |
| 97 | 3544 | CGATCAAGCTACTTTATGT | 96 |
| 98 | 2839 | CCTGGTATGAATAGACAGA | 97 |
| 99 | 2943 | CAGTAATACATTCCATTGT | 98 |
| 100 | 1758 | GTGAACCTTTGAGCTTTCA | 99 |
| 101 | 175 | GCTGAAAATGACTGAATAT | 101 |
| 102 | 176 | CTGAAAATGACTGAATATA | 102 |
| 103 | 178 | GAAAATGACTGAATATAAA | 103 |
| 104 | 240 | CGATACAGCTAATTCAGAA | 104 |
| 105 | 245 | CAGCTAATTCAGAATCATT | 105 |
| 106 | 247 | GCTAATTCAGAATCATTTT | 106 |
| 107 | 256 | GAATCATTTTGTGGACGAA | 107 |
| 108 | 271 | CGAATATGATCCAACAATA | 108 |
| 109 | 278 | GATCCAACAATAGAGGATT | 109 |
| 110 | 282 | CAACAATAGAGGATTCCTA | 110 |
| 111 | 292 | GGATTCCTACAGGAAGCAA | 111 |
| 112 | 297 | CCTACAGGAAGCAAGTAGT | 112 |
| 113 | 298 | CTACAGGAAGCAAGTAGTA | 113 |
| 114 | 301 | CAGGAAGCAAGTAGTAATT | 114 |
| 115 | 307 | GCAAGTAGTAATTGATGGA | 115 |
| 116 | 311 | GTAGTAATTGATGGAGAAA | 116 |
| 117 | 320 | GATGGAGAAACCTGTCTCT | 117 |
| 118 | 324 | GAGAAACCTGTCTCTTGGA | 118 |
| 119 | 326 | GAAACCTGTCTCTTGGATA | 119 |
| 120 | 333 | GTCTCTTGGATATTCTCGA | 120 |
| 121 | 335 | CTCTTGGATATTCTCGACA | 121 |
| 122 | 337 | CTTGGATATTCTCGACACA | 122 |
| 123 | 340 | GGATATTCTCGACACAGCA | 123 |
| 124 | 347 | CTCGACACAGCAGGTCAAG | 124 |
| 125 | 356 | GCAGGTCAAGAGGAGTACA | 125 |
| 126 | 362 | CAAGAGGAGTACAGTGCAA | 126 |
| 127 | 365 | GAGGAGTACAGTGCAATGA | 127 |
| 128 | 377 | CAATGAGGGACCAGTACA | 128 |
| 129 | 385 | GGACCAGTACATGAGGACT | 129 |
| 130 | 405 | GGGAGGGCTTTCTTTGTGT | 130 |
| 131 | 407 | GAGGGCTTTCTTTGTGTAT | 131 |
| 132 | 409 | GGGCTTTCTTTGTGTATTT | 132 |
| 133 | 416 | CTTTGTGTATTTGCCATAA | 133 |
| 134 | 422 | GTATTTGCCATAAATAAT | 134 |
| 135 | 441 | CTAAATCATTTGAAGATAT | 135 |
| 136 | 452 | GAAGATATTCACCATTATA | 136 |
| 137 | 463 | CCATTATAGAGAACAAATT | 137 |
| 138 | 464 | CATTATAGAGAACAAATTA | 138 |
| 139 | 471 | GAGAACAAATTAAAAGAGT | 139 |
| 140 | 473 | GAACAAATTAAAAGAGTTA | 140 |
| 141 | 486 | GAGTTAAGGACTCTGAAGA | 141 |
| 142 | 488 | GTTAAGGACTCTGAAGATG | 142 |
| 143 | 493 | GGACTCTGAAGATGTACCT | 143 |
| 144 | 494 | GACTCTGAAGATGTACCTA | 144 |
| 145 | 498 | CTGAAGATGTACCTATGGT | 145 |
| 146 | 509 | CCTATGGTCCTAGTAGGAA | 146 |
| 147 | 510 | CTATGGTCCTAGTAGGAAA | 147 |
| 148 | 515 | GTCCTAGTAGGAAATAAAT | 148 |
| 149 | 521 | GTAGGAAATAAATGTGATT | 149 |
| 150 | 542 | CCTTCTAGAACAGTAGACA | 150 |
| 151 | 546 | CTAGAACAGTAGACACAAA | 151 |
| 152 | 549 | GAACAGTAGACACAAAACA | 152 |
| 153 | 561 | CAAAACAGGCTCAGGACTT | 153 |
| 154 | 566 | CAGGCTCAGGACTTAGCAA | 154 |
| 155 | 568 | GGCTCAGGACTTAGCAAGA | 155 |
| 156 | 572 | CAGGACTTAGCAAGAAGTT | 156 |
| 157 | 577 | CTTAGCAAGAAGTTATGGA | 157 |
| 158 | 581 | GCAAGAAGTTATGGAATTC | 158 |
| 159 | 585 | GAAGTTATGGAATTCCTTT | 159 |
| 160 | 588 | GTTATGGAATTCCTTTTAT | 160 |
| 161 | 593 | GGAATTCCTTTTATTGAAA | 161 |
| 162 | 608 | GAAACATCAGCAAAGACAA | 162 |
| 163 | 612 | CATCAGCAAAGACAAGACA | 163 |
| 164 | 618 | GCAAAGACAAGACAGGGTG | 164 |
| 165 | 619 | CAAAGACAAGACAGGGTGT | 165 |
| 166 | 622 | GACAAGACAGGGTGTTGAT | 166 |
| 167 | 624 | CAAGACAGGGTGTTGATGA | 167 |
| 168 | 629 | CAGGGTGTTGATGATGCCT | 168 |
| 169 | 632 | GGTGTTGATGATGCCTTCT | 169 |
| 170 | 633 | GTGTTGATGATGCCTTCTA | 170 |
| 171 | 635 | GTTGATGATGCCTTCTATA | 171 |
| 172 | 639 | ATGATGCCTTCTATACATT | 172 |
| 173 | 641 | GATGCCTTCTATACATTAG | 173 |
| 174 | 644 | GCCTTCTATACATTAGTTC | 174 |
| 175 | 646 | CTTCTATACATTAGTTCGA | 175 |
| 176 | 649 | CTATACATTAGTTCGAGAA | 176 |
| 177 | 662 | CGAGAAATTCGAAAACATA | 177 |
| 178 | 663 | GAGAAATTCGAAAACATAA | 178 |
| 179 | 671 | CGAAAACATAAAGAAAAGA | 179 |
| 180 | 672 | GAAAACATAAAGAAAAGAT | 180 |
| 181 | 677 | CATAAAGAAAAGATGAGCA | 181 |
| 182 | 693 | GCAAAGATGGTAAAAAGAA | 182 |
| 183 | 694 | CAAAGATGGTAAAAAGAAG | 183 |
| 184 | 698 | GATGGTAAAAAGAAGAAAA | 184 |
| 185 | 701 | GGTAAAAAGAAGAAAAAGA | 185 |
| 186 | 702 | GTAAAAAGAAGAAAAAGAA | 186 |
| 187 | 709 | GAAGAAAAAGAAGTCAAAG | 187 |
| 188 | 712 | GAAAAAGAAGTCAAAGACA | 188 |
| 189 | 718 | GAAGTCAAAGACAAAGTGT | 189 |
| 190 | 721 | GTCAAAGACAAAGTGTGTA | 190 |
| 191 | 723 | CAAAGACAAAGTGTGTAAT | 191 |
| 192 | 727 | GACAAAGTGTGTAATTATG | 192 |
| 193 | 729 | CAAAGTGTGTAATTATGTA | 193 |
| 194 | 752 | CAATTTGTACTTTTTTCTT | 194 |
| 195 | 758 | GTACTTTTTTCTTAAGGCA | 195 |
| 196 | 761 | CTTTTTTCTTAAGGCATAC | 196 |
| 197 | 768 | CTTAAGGCATACTAGTACA | 197 |
| 198 | 775 | CATACTAGTACAAGTGGTA | 198 |
| 199 | 779 | CTAGTACAAGTGGTAATTT | 199 |
| 200 | 782 | GTACAAGTGGTAATTTTTG | 200 |
| 201 | 788 | GTGGTAATTTTTGTACATT | 201 |
| 202 | 791 | GTAATTTTTGTACATTACA | 202 |
| 203 | 800 | GUACAUUACACUAAAUUAU | 203 |
| 204 | 808 | CATTACACTAAATTATTAG | 204 |
| 205 | 810 | CTAAATTATTAGCATTTGT | 205 |
| 206 | 821 | GCATTTGTTTTAGCATTAC | 206 |
| 207 | 827 | GTTTTAGCATTACCTAATT | 207 |
| 208 | 851 | CCTGCTCCATGCAGACTGT | 208 |
| 209 | 852 | CTGCTCCATGCAGACTGTT | 209 |
| 210 | 854 | GCTCCATGCAGACTGTTAG | 210 |
| 211 | 857 | CCATGCAGACTGTTAGCTT | 211 |
| 212 | 862 | GACTGTTAGCTTTTACCTTA | 212 |
| 213 | 868 | GUUAGCUUUUACCUUAAAU | 213 |
| 214 | 872 | GCUUUUACCUUAAAUGCUU | 214 |
| 215 | 873 | CUUUUACCUUAAAUGCUUA | 215 |
| 216 | 911 | GUUUUUUUUUCCUCUAAGU | 216 |
| 217 | 931 | CCAGUAUUCCCAGAGUUUU | 217 |
| 218 | 941 | CAGAGUUUUGGUUUUUGAA | 218 |
| 219 | 943 | GAGUUUUGGUUUUUGAACU | 219 |
| 220 | 960 | CUAGCAAUGCCUGUGAAAA | 220 |
| 221 | 970 | CUGUGAAAAAGAAACUGAA | 221 |
| 222 | 972 | GUGAAAAAGAAACUGAAUA | 222 |
| 223 | 984 | CUGAAUACCUAAGAUUUCU | 223 |
| 224 | 986 | GAAUACCUAAGAUUUCUGU | 224 |
| 225 | 1025 | CAGUUGAUUACUUCUUAUU | 225 |
| 226 | 1027 | GUUGAUUACUUCUUAUUUU | 226 |
| 227 | 1030 | GAUUACUUCUUAUUUUUCU | 227 |
| 228 | 1038 | CUUAUUUUUCUUACCAAUU | 228 |
| 229 | 1047 | CUUACCAAUUGUGAAUGUU | 229 |
| 230 | 1059 | GAAUGUUGGUGUGAAACAA | 230 |
| 231 | 1067 | GUGUGAAACAAAUUAAUGA | 231 |
| 232 | 1101 | CCUAUUCUGUGUUUUAUCU | 232 |
| 233 | 1102 | CUAUUCUGUGUUUUAUCUA | 233 |
| 234 | 1125 | CAUAAAUGGAUUAAUUACU | 234 |
| 235 | 1159 | CUUCUAAUUGGUUUUUACU | 235 |
| 236 | 1162 | CUAAUUGGUUUUUACUGAA | 236 |
| 237 | 1169 | GUUUUUACUGAAACAUUGA | 237 |
| 238 | 1230 | GCAUCAUGUCCUAUAGUUU | 238 |
| 239 | 1278 | GUUCACAAAGGUUUUGUCU | 239 |
| 240 | 1403 | GCCAUUUCCUUUUCACAUU | 240 |
| 241 | 1404 | CCAUUUCCUUUUCACAUUA | 241 |
| 242 | 855 | CTCCATGCAGACTGTTAGC | 242 |
| 243 | 858 | CATGCAGACTGTTAGCTTT | 243 |
| 244 | 861 | GCAGACTGTTAGCTTTTAC | 244 |
| 245 | 866 | CTGTTAGCTTTTACCTTAA | 245 |
| 246 | 879 | CCTTAAATGCTTATTTTAA | 246 |
| 247 | 901 | GACAGTGGAAGTTTTTTTT | 247 |
| 248 | 902 | ACAGTGGAAGTTTTTTTTT | 248 |
| 249 | 921 | CCTCTAAGTGCCAGTATTC | 249 |
| 250 | 924 | CTAAGTGCCAGTATTCCCA | 250 |
| 251 | 928 | GTGCCAGTATTCCCAGAGT | 251 |
| 252 | 930 | GCCAGTATTCCCAGAGTTT | 252 |
| 253 | 931 | CCAGTATTCCCAGAGTTTT | 253 |
| 254 | 932 | CAGTATTCCCAGAGTTTTG | 254 |
| 255 | 934 | GTATTCCCAGAGTTTTGGT | 255 |
| 256 | 939 | CCCAGAGTTTTGGTTTTTG | 256 |
| 257 | 940 | CCAGAGTTTTGGTTTTTGA | 257 |
| 258 | 942 | AGAGTTTTGGTTTTTGAAC | 258 |
| 259 | 945 | GTTTTGGTTTTTGAACTAG | 259 |
| 260 | 950 | GGTTTTTGAACTAGCAATG | 260 |
| 261 | 957 | GAACTAGCAATGCCTGTGA | 261 |
| 262 | 963 | GCAATGCCTGTGAAAAAGA | 262 |
| 263 | 964 | CAATGCCTGTGAAAAAGAA | 263 |
| 264 | 968 | GCCTGTGAAAAAGAAACTG | 264 |
| 265 | 969 | CCTGTGAAAAAGAAACTGA | 265 |
| 266 | 973 | TGAAAAAGAAACTGAATAC | 266 |
| 267 | 980 | GAAACTGAATACCTAAGAT | 267 |
| 268 | 1001 | CTGTCTTGGGGTTTTTGGT | 268 |
| 269 | 1003 | GTCTTGGGGTTTTTGGTGC | 269 |
| 270 | 1005 | CTTGGGGTTTTTGGTGCAT | 270 |
| 271 | 1010 | GCATGCAGTGTTTTTGGTG | 271 |
| 272 | 1011 | GTTTTTGGTGCATGCAGTT | 272 |
| 273 | 1410 | CCTTTTCACATTAGATAAA | 273 |
| 274 | 1411 | CTTTTCACATTAGATAAAT | 274 |
| 275 | 1474 | GTATGAAATGGGGATTATT | 275 |
| 276 | 1450 | CCATTTTGGGGCTATATTT | 276 |
| 277 | 1451 | CATTTTGGGGCTATATTTA | 277 |
| 278 | 1546 | GAAAAGATTTTAACAAGTA | 278 |
| 279 | 1559 | CAAGTATAAAAAATTCTCA | 279 |
| 280 | 1576 | CATAGGAATTAAATGTAGT | 280 |
| 281 | 1611 | GCTCTTTCATAGTATAACT | 281 |
| 282 | 1612 | CTCTTTCATAGTATAACTT | 282 |
| 283 | 1614 | CTTTCATAGTATAACTTTA | 283 |
| 284 | 1628 | CTTTAAATCTTTTCTTCAA | 284 |
| 285 | 1641 | CTTCAACTTGAGTCTTTGA | 285 |
| 286 | 1644 | CAACTTGAGTCTTTGAAGA | 286 |
| 287 | 1650 | GAGTCTTTGAAGATAGTTT | 287 |
| 288 | 1652 | GTCTTTGAAGATAGTTTTA | 288 |
| 289 | 1654 | CTTTGAAGATAGTTTTAAT | 289 |
| 290 | 1704 | CAGTTATAGCTTATTAGGT | 290 |
| 291 | 1712 | GCTTATTAGGTGTTGAAGA | 291 |
| 292 | 1770 | GCTTTCATAGAGAGTTTCA | 292 |
| 293 | 1826 | CATGCATTGGTTAGTCAAA | 293 |
| 294 | 1925 | CATTGCTTTTGTTTCTTAA | 294 |
| 295 | 1929 | GCTTTTGTTTCTTAAGAAA | 295 |
| 296 | 1930 | CTTTTGTTTCTTAAGAAAA | 296 |
| 297 | 1939 | CTTAAGAAAACAAACTCTT | 297 |
| 298 | 1944 | GAAAACAAACTCTTTTTTA | 298 |
| 299 | 2041 | CAATGAAGTGAAAAAGTTT | 299 |
| 300 | 2045 | GAAGTGAAAAAGTTTTACA | 300 |
| 301 | 2084 | CTCTTAACACTGGTTAAAT | 301 |
| 302 | 2086 | CTTAACACTGGTTAAATTA | 302 |
| 303 | 2096 | GTTAAATTAACATTGCATA | 303 |
| 304 | 2110 | GCATAAACACTTTTCAAGT | 304 |
| 305 | 2169 | CAATCCTTTTGATAAATTT | 305 |
| 306 | 2263 | GTGACTTAGGTTCTAGATA | 306 |
| 307 | 2287 | CTTTTAGGACTCTGATTTT | 307 |
| 308 | 2311 | CATCACTTACTATCCATTT | 308 |
| 309 | 2314 | CACTTACTATCCATTTCTT | 309 |
| 310 | 2316 | CTTACTATCCATTTCTTCA | 310 |
| 311 | 2320 | CTATCCATTTCTTCATGTT | 311 |
| 312 | 2324 | CCATTTCTTCATGTTAAAA | 312 |
| 313 | 2343 | GAAGTCATCTCAAACTCTT | 313 |
| 314 | 2348 | CATCTCAAACTCTTAGTTT | 314 |
| 315 | 2351 | CTCAAACTCTTAGTTTTTT | 315 |
| 316 | 2380 | CTATGTAATTTATATTCCA | 316 |
| 317 | 2403 | CATAAGGATACACTTATTT | 317 |
| 318 | 2432 | GCACAATCTGTAAATTTTT | 318 |
| 319 | 2454 | CTATGTTACACCATCTTCA | 319 |

In some embodiments, the RNA duplex molecule, also referred to herein as an asymmetrical interfering RNA molecule or aiRNA molecule, comprises a sense strand sequence, an antisense strand sequence or a combination of a sense strand sequence and antisense strand sequence selected from those shown in Table 2 below.

TABLE 2

| | | | | Antisense |
|---|---|---|---|---|
| aiRNA | Sense Strand | Sense Strand | Antisense Strand | Strand SEQ |
| ID NO: | Sequence | SEQ ID NO: | Sequence | ID NO: |
| 1 | CAGUUAUAGCUUAUU | 320 | AAUAAUAAGCUAUAACUGGCC | 638 |
| 2 | CCUAGUAGGAAAUAA | 321 | AAUUUAUUUCCUACUAGGACC | 639 |
| 3 | AGACCCAGUAUGAAA | 322 | AAAUUUCAUACUGGGUCUGCC | 640 |
| 4 | GUGCCAAGACAUUAA | 323 | AAAUUAAUGUCUUGGCACACC | 641 |
| 5 | CUCUUCUUCCAUAUU | 324 | AAUAAUAUGGAAGAAGAGUCC | 642 |
| 6 | AAUGGAAACUAUUAU | 325 | AAUAUAAUAGUUUCCAUUGCC | 643 |
| 7 | GUUGAUUACUUCUUA | 326 | AAAUAAGAAGUAAUCAACUGC | 644 |
| 8 | CUUAGCAAGAAGUUA | 327 | AAAUAACUUCUUGCUAAGUCC | 645 |
| 9 | CAGCACAAUCUGUAA | 328 | AAUUUACAGAUUGUGCUGAGC | 646 |
| 10 | CUUUCCACUGCUAUU | 329 | AAUAAUAGCAGUGGAAAGGAG | 647 |
| 11 | GGUGUGAAACAAAUU | 330 | AAUAAUUUGUUUCACACCAAC | 648 |
| 12 | UACAGCUAAUUCAGA | 331 | AAUUCUGAAUUAGCUGUAUCG | 649 |
| 13 | CUAAUUCAGAAUCAU | 332 | AAAAUGAUUCUGAAUUAGCUG | 650 |
| 14 | AAUUCAGAAUCAUUU | 333 | AAAAAAUGAUUCUGAAUUAGC | 651 |
| 15 | AUAUGAUCCAACAAU | 334 | AAUAUUGUUGGAUCAUAUUCG | 652 |
| 16 | GGUAACAGUAAUACA | 335 | AAAUGUAUUACUGUUACCAGG | 653 |
| 17 | CAGGACUUAGCAAGA | 336 | AAUUCUUGCUAAGUCCUGAGC | 654 |
| 18 | UGUGCCAGCUCUAUA | 337 | AAUUAUAGAGCUGGCACAGAG | 655 |
| 19 | CUAUAUUUACAUGCU | 338 | AAUAGCAUGUAAAUAUAGCCC | 656 |
| 20 | GUCUCUUGGAUAUUC | 339 | AAAGAAUAUCCAAGAGACAGG | 657 |
| 21 | GGGCUUUCUUUGUGU | 340 | AAUACACAAAGAAAGCCCUCC | 658 |
| 22 | GAUAUCUCCAUGAAG | 341 | AAACUUCAUGGAGAUAUCCAC | 659 |
| 23 | CAUUAUAGAGAACAA | 342 | AAUUUGUUCUCUAUAAUGGUG | 660 |
| 24 | CUGUAAUAUCUUACU | 343 | AAUAGUAAGAUAUUACAGACC | 661 |
| 25 | UGACGAUACAGCUAA | 344 | AAAUUAGCUGUAUCGUCAAGG | 662 |
| 26 | GAUGCUUUGAACAUC | 345 | AAAGAUGUUCAAAGCAUCAGC | 663 |
| 27 | CCCUGAUGAAUGUAA | 346 | AAUUUACAUUCAUCAGGGAUG | 664 |
| 28 | UAUUUGCCAUAAAUA | 347 | AAUUAUUUAUGGCAAAUACAC | 665 |
| 29 | AAAUAAUACUAAAUC | 348 | AAUGAUUUAGUAUUAUUUAUG | 666 |
| 30 | AAUCAUUUGAAGAUA | 349 | AAAUAUCUUCAAAUGAUUUAG | 667 |
| 31 | GAUAUUCACCAUUAU | 350 | AAUAUAAUGGUGAAUAUCUUC | 668 |
| W32 | UUUAACAAAAGAUUU | 351 | AACAAAUCUUUUGUUAAACCA | 669 |
| W33 | CCUAAUAUAUACAUA | 352 | AACUAUGUAUAUAUUAGGACA | 670 |
| W34 | AAAAGAAACUGAAUA | 353 | AAGUAUUCAGUUUCUUUUUCA | 671 |
| 35 | AGCACAAUCUGUAAA | 354 | AAAUUUACAGAUUGUGCUGAG | 672 |
| 36 | CUUUCAUAGUAUAAC | 355 | AAAGUUAUACUAUGAAAGAGC | 673 |
| 37 | CUAGUGUGGUCUGUA | 356 | AAUUACAGACCACACUAGCAC | 674 |
| 38 | GUGUGGUCUGUAAUA | 357 | AAAUAUUACAGACCACACUAG | 675 |
| 39 | GACGUAUAUUGUAUC | 358 | AAUGAUACAAUAUACGUCUGC | 676 |
| 40 | CCCAAGUAGGCAUUC | 359 | AAAGAAUGCCUACUUGGGAAC | 677 |
| 41 | CGAAUAUGAUCCAAC | 360 | AAUGUUGGAUCAUAUUCGUCC | 678 |
| 42 | GCAAGUAGUAAUUGA | 361 | AAAUCAAUUACUACUUGCUUC | 679 |
| 43 | UCCUGAUGAUGAUUC | 362 | AAAGAAUCAUCAUCAGGAAGC | 680 |
| 44 | GACCUCAAGUGAUUC | 363 | AAUGAAUCACUUGAGGUCAGG | 681 |
| W45 | UCCCUACCUUCCACA | 364 | AAAUGUGGAAGGUAGGGAGGC | 682 |
| W46 | AUUUCCUUUUCACAU | 365 | AAAAUGUGAAAAGGAAAUGGC | 683 |
| W47 | GUAUAUUGUAUCAUU | 366 | AAAAAUGAUACAAUAUACGUC | 684 |
| W48 | CAUUUCCUUUUCACA | 367 | AAAUGUGAAAAGGAAAUGGCC | 685 |
| W49 | GGAGAAUUCUAGAAA | 368 | AAAUUUCUAGAAUUCUCCCCC | 686 |
| 50 | CCCAAGUAGGCAUUC | 369 | AAAGAAUGCCUACUUGGGAAC | 687 |
| 51 | CGAAUAUGAUCCAAC | 370 | AAUGUUGGAUCAUAUUCGUCC | 688 |
| 52 | GCAAGUAGUAAUUGA | 371 | AAAUCAAUUACUACUUGCUUC | 689 |
| 53 | UCCUGAUGAUGAUUC | 372 | AAAGAAUCAUCAUCAGGAAGC | 690 |
| 54 | CUGUACUACUCCUAA | 373 | AAAUUAGGAGUAGUACAGUUC | 691 |
| 55 | GACCUCAAGUGAUUC | 374 | AAUGAAUCACUUGAGGUCAGG | 692 |
| 56 | CUUAGGUAGUGCUAG | 375 | AAACUAGCACUACCUAAGGAC | 693 |
| 57 | UCAGACUGCUCUUUC | 376 | AAUGAAAGAGCAGUCUGACAC | 694 |
| 58 | UGCUCUUUCAUAGUA | 377 | AAAUACUAUGAAAGAGCAGUC | 695 |
| 59 | GAUGAAUGUAAAGUU | 378 | AAUAACUUUACAUUCAUCAGG | 696 |
| 60 | GUCUGAUCCAUAUUU | 379 | AAUAAAUAUGGAUCAGACUUG | 697 |
| 61 | GAGCAAAGAUGGUAA | 380 | AAUUUACCAUCUUUGCUCAUC | 698 |
| 62 | GAGGUGAAGUUUAUA | 381 | AAAUAUAAACUUCACCUCUUG | 699 |
| 63 | AGGGUGUUAAGACUU | 382 | AAUAAGUCUUAACACCCUACC | 700 |
| 64 | GGCAUCAUGUCCUAU | 383 | AAUAUAGGACAUGAUGCCUAG | 701 |
| 65 | UGAAUGUUCCCAAGU | 384 | AAUACUUGGGAACAUUCACUC | 702 |
| 66 | AGUAGGAAAUAAAUG | 385 | AAACAUUUAUUUCCUACUAGG | 703 |
| 67 | UGAACUAGUUCACAG | 386 | AAUCUGUGAACUAGUUCAGGC | 704 |
| 68 | GAAAUCUUCAUGCAA | 387 | AAAUUGCAUGAAGAUUUCUGG | 705 |
| 69 | GUGGAUAUCUCCAUG | 388 | AAUCAUGGAGAUAUCCACAGC | 706 |
| 70 | AGCAAGUAGUAAUUG | 389 | AAUCAAUUACUACUUGCUUCC | 707 |
| 71 | ACGUAUAUUGUAUCA | 390 | AAAUGAUACAAUAUACGUCUG | 708 |
| 72 | UUGACGAUACAGCUA | 391 | AAUUAGCUGUAUCGUCAAGGC | 709 |
| 73 | GGUGACUUAGGUUCU | 392 | AAUAGAACCUAAGUCACCUUC | 710 |
| 74 | UAGUUCUCUUAACAC | 393 | AAAGUGUUAAGAGAACUAGCC | 711 |
| 75 | UAUGUCAGAUAUUCA | 394 | AAAUGAAUAUCUGACAUACAC | 712 |
| 76 | CCUUUGAGCUUUCAU | 395 | AAUAUGAAAGCUCAAAGGUUC | 713 |
| 77 | ACAAGGAAACUUCUA | 396 | AAAUAGAAGUUUCCUUGUCUG | 714 |
| 78 | CGAUCAAGCUACUUU | 397 | AAUAAAGUAGCUUGAUCGAAG | 715 |
| 79 | UGCCAAUUUCUUACU | 398 | AAUAGUAAGAAAUUGGCACUC | 716 |
| 80 | GACAAAUCAAGAGCA | 399 | AAAUGCUCUUGAUUUGUCAGC | 717 |
| 81 | AUCUCAAACUCUUAG | 400 | AAACUAAGAGUUUGAGAUGAC | 718 |
| 82 | GAUGCCUUCUAUACA | 401 | AAAUGUAUAGAAGGCAUCAUC | 719 |
| 83 | GUAUGAAUAGACAGA | 402 | AAUUCUGUCUAUUCAUACCAG | 720 |
| 84 | UGAGUCACAUCAGAA | 403 | AAUUUCUGAUGUGACUCAGUG | 721 |
| 85 | GUCACCAUUGCACAA | 404 | AAAUUGUGCAAUGGUGACAAC | 722 |
| 86 | AAGCUCAGCACAAUC | 405 | AAAGAUUGUGCUGAGCUUGAC | 723 |
| 87 | AUUAUUAUAGCAACC | 406 | AAUGGUUGCUAUAAUAAUCCC | 724 |
| 88 | GGAAGAAGGUGACUU | 407 | AAUAAGUCACCUUCUUCCUAG | 725 |
| 89 | CUCUGAAGAUGUACC | 408 | AAAGGUACAUCUUCAGAGUCC | 726 |
| 90 | AGUAGCUGGGAUUAC | 409 | AAUGUAAUCCCAGCUACUCAG | 727 |
| 91 | GAGUUCUUGAAGAAU | 410 | AAUAUUCUUCAAGAACUCAUG | 728 |
| 92 | GACGAAUAUGAUCCA | 411 | AAUUGGAUCAUAUUCGUCCAC | 729 |
| 93 | UGGAUAUCUCCAUGA | 412 | AAUUCAUGGAGAUAUCCACAG | 730 |
| 94 | AAGGAAACUUCUAUG | 413 | AAACAUAGAAGUUUCCUUGUC | 731 |
| 95 | UAUAGCAGACGUAUA | 414 | AAAUAUACGUCUGCUAUAUUC | 732 |
| 96 | UCAAGCUACUUUAUG | 415 | AAACAUAAAGUAGCUUGAUCG | 733 |
| 97 | GGUAUGAAUAGACAG | 416 | AAUCUGUCUAUUCAUACCAGG | 734 |
| 98 | UAAUACAUUCCAUUG | 417 | AAACAAUGGAAUGUAUUACUG | 735 |
| 99 | AACCUUUGAGCUUUC | 418 | AAUGAAAGCUCAAAGGUUCAC | 736 |
| 101 | GAAAAUGACUGAAUA | 419 | AAAUAUUCAGUCAUUUUCAGC | 737 |
| 102 | AAAAUGACUGAAUAU | 420 | AAUAUAUUCAGUCAUUUUCAG | 738 |
| 103 | AAUGACUGAAUAUAA | 421 | AAUUUAUAUUCAGUCAUUUUC | 739 |
| 104 | UACAGCUAAUUCAGA | 422 | AAUUCUGAAUUAGCUGUAUCG | 740 |
| 105 | CUAAUUCAGAAUCAU | 423 | AAAAUGAUUCUGAAUUAGCUG | 741 |
| 106 | AAUUCAGAAUCAUUU | 424 | AAAAAAUGAUUCUGAAUUAGC | 742 |
| 107 | UCAUUUUGUGGACGA | 425 | AAUUCGUCCACAAAAUGAUUC | 743 |
| 108 | AUAUGAUCCAACAAU | 426 | AAUAUUGUUGGAUCAUAUUCG | 744 |
| 109 | CCAACAAUAGAGGAU | 427 | AAAAUCCUCUAUUGUUGGAUC | 745 |
| 110 | CAAUAGAGGAUUCCU | 428 | AAUAGGAAUCCUCUAUUGUUG | 746 |
| 111 | UUCCUACAGGAAGCA | 429 | AAUUGCUUCCUGUAGGAAUCC | 747 |
| 112 | ACAGGAAGCAAGUAG | 430 | AAACUACUUGCUUCCUGUAGG | 748 |
| 113 | CAGGAAGCAAGUAGU | 431 | AAUACUACUUGCUUCCUGUAG | 749 |
| 114 | GAAGCAAGUAGUAAU | 432 | AAAAUUACUACUUGCUUCCUG | 750 |
| 115 | AGUAGUAAUUGAUGG | 433 | AAUCCAUCAAUUACUACUUGC | 751 |
| 116 | GUAAUUGAUGGAGAA | 434 | AAUUUCUCCAUCAAUUACUAC | 752 |
| 117 | GGAGAAACCUGUCUC | 435 | AAAGAGACAGGUUUCUCCAUC | 753 |
| 118 | AAACCUGUCUCUUGG | 436 | AAUCCAAGAGACAGGUUUCUC | 754 |
| 119 | ACCUGUCUCUUGGAU | 437 | AAUAUCCAAGAGACAGGUUUC | 755 |
| 120 | UCUUGGAUAUUCUCG | 438 | AAUCGAGAAUAUCCAAGAGAC | 756 |
| 121 | UUGGAUAUUCUCGAC | 439 | AAUGUCGAGAAUAUCCAAGAG | 757 |
| 122 | GGAUAUUCUCGACAC | 440 | AAUGUGUCGAGAAUAUCCAAG | 758 |
| 123 | UAUUCUCGACACAGC | 441 | AAUGCUGUGUCGAGAAUAUCC | 759 |
| 124 | GACACAGCAGGUCAA | 442 | AACUUGACCUGCUGUGUCGAG | 760 |
| 125 | GGUCAAGAGGAGUAC | 443 | AAUGUACUCCUCUUGACCUGC | 761 |
| 126 | GAGGAGUACAGUGCA | 444 | AAUUGCACUGUACUCCUCUUG | 762 |
| 127 | GAGUACAGUGCAAUG | 445 | AAUCAUUGCACUGUACUCCUC | 763 |
| 128 | UGAGGGACCAGUAC | 446 | AAUGUACUGGUCCCUCAUUG | 764 |
| 129 | CCAGUACAUGAGGAC | 447 | AAAGUCCUCAUGUACUGGUCC | 765 |
| 130 | AGGGCUUUCUUUGUG | 448 | AAACACAAAGAAAGCCCUCCC | 766 |
| 131 | GGCUUUCUUUGUGUA | 449 | AAAUACACAAAGAAAGCCCUC | 767 |
| 132 | CUUUCUUUGUGUAUU | 450 | AAAAAUACACAAAGAAAGCCC | 768 |
| 133 | UGUGUAUUUGCCAUA | 451 | AAUUAUGGCAAAUACACAAAG | 769 |
| 134 | UUUGCCAUAAAUAA | 452 | AAAUUAUUUAUGGCAAAUAC | 770 |
| 135 | AAUCAUUUGAAGAUA | 453 | AAAUAUCUUCAAAUGAUUUAG | 771 |
| 136 | GAUAUUCACCAUUAU | 454 | AAUAUAAUGGUGAAUAUCUUC | 772 |
| 137 | UUAUAGAGAACAAAU | 455 | AAAAUUUGUUCUCUAUAAUGG | 773 |
| 138 | UAUAGAGAACAAAUU | 456 | AAUAAUUUGUUCUCUAUAAUG | 774 |
| 139 | AACAAAUUAAAAGAG | 457 | AAACUCUUUUAAUUUGUUCUC | 775 |
| 140 | CAAAUUAAAAGAGUU | 458 | AAUAACUCUUUUAAUUUGUUC | 776 |
| 141 | UUAAGGACUCUGAAG | 459 | AAUCUUCAGAGUCCUUAACUC | 777 |
| 142 | AAGGACUCUGAAGAU | 460 | AACAUCUUCAGAGUCCUUAAC | 778 |
| 143 | CUCUGAAGAUGUACC | 461 | AAAGGUACAUCUUCAGAGUCC | 779 |
| 144 | UCUGAAGAUGUACCU | 462 | AAUAGGUACAUCUUCAGAGUC | 780 |
| 145 | AAGAUGUACCUAUGG | 463 | AAACCAUAGGUACAUCUUCAG | 781 |
| 146 | AUGGUCCUAGUAGGA | 464 | AAUUCCUACUAGGACCAUAGG | 782 |
| 147 | UGGUCCUAGUAGGAA | 465 | AAUUUCCUACUAGGACCAUAG | 783 |
| 148 | CUAGUAGGAAAUAAA | 466 | AAAUUUAUUUCCUACUAGGAC | 784 |
| 149 | GGAAAUAAAUGUGAU | 467 | AAAAUCACAUUUAUUUCCUAC | 785 |
| 150 | UCUAGAACAGUAGAC | 468 | AAUGUCUACUGUUCUAGAAGG | 786 |
| 151 | GAACAGUAGACACAA | 469 | AAUUUGUGUCUACUGUUCUAG | 787 |
| 152 | CAGUAGACACAAAAC | 470 | AAUGUUUUGUGUCUACUGUUC | 788 |
| 153 | AACAGGCUCAGGACU | 471 | AAAAGUCCUGAGCCUGUUUUG | 789 |
| 154 | GCUCAGGACUUAGCA | 472 | AAUUGCUAAGUCCUGAGCCUG | 790 |
| 155 | UCAGGACUUAGCAAG | 473 | AAUCUUGCUAAGUCCUGAGCC | 791 |
| 156 | GACUUAGCAAGAAGU | 474 | AAAACUUCUUGCUAAGUCCUG | 792 |
| 157 | AGCAAGAAGUUAUGG | 475 | AAUCCAUAACUUCUUGCUAAG | 793 |
| 158 | AGAAGUUAUGGAAUU | 476 | AAGAAUUCCAUAACUUCUUGC | 794 |
| 159 | GUUAUGGAAUUCCUU | 477 | AAAAAGGAAUUCCAUAACUUC | 795 |
| 160 | AUGGAAUUCCUUUUA | 478 | AAAUAAAAGGAAUUCCAUAAC | 796 |
| 161 | AUUCCUUUUAUUGAA | 479 | AAUUUCAAUAAAAGGAAUUCC | 797 |
| 162 | ACAUCAGCAAAGACA | 480 | AAUUGUCUUUGCUGAUGUUUC | 798 |
| 163 | CAGCAAAGACAAGAC | 481 | AAUGUCUUGUCUUUGCUGAUG | 799 |
| 164 | AAGACAAGACAGGGU | 482 | AACACCCUGUCUUGUCUUUGC | 800 |
| 165 | AGACAAGACAGGGUG | 483 | AAACACCCUGUCUUGUCUUUG | 801 |
| 166 | AAGACAGGGUGUUGA | 484 | AAAUCAACACCCUGUCUUGUC | 802 |
| 167 | GACAGGGUGUUGAUG | 485 | AAUCAUCAACACCCUGUCUUG | 803 |
| 168 | GGUGUUGAUGAUGCC | 486 | AAAGGCAUCAUCAACACCCUG | 804 |
| 169 | GUUGAUGAUGCCUUC | 487 | AAAGAAGGCAUCAUCAACACC | 805 |
| 170 | UUGAUGAUGCCUUCU | 488 | AAUAGAAGGCAUCAUCAACAC | 806 |
| 171 | GAUGAUGCCUUCUAU | 489 | AAUAUAGAAGGCAUCAUCAAC | 807 |
| 172 | AUGCCUUCUAUACAU | 490 | AAAAUGUAUAGAAGGCAUCAU | 808 |
| 173 | GCCUUCUAUACAUUA | 491 | AACUAAUGUAUAGAAGGCAUC | 809 |
| 174 | UUCUAUACAUUAGUU | 492 | AAGAACUAAUGUAUAGAAGGC | 810 |
| 175 | CUAUACAUUAGUUCG | 493 | AAUCGAACUAAUGUAUAGAAG | 811 |
| 176 | UACAUUAGUUCGAGA | 494 | AAUUCUCGAACUAAUGUAUAG | 812 |
| 177 | GAAAUUCGAAAACAU | 495 | AAUAUGUUUUCGAAUUUCUCG | 813 |
| 178 | AAAUUCGAAAACAUA | 496 | AAUUAUGUUUUCGAAUUUCUC | 814 |
| 179 | AAACAUAAAGAAAAG | 497 | AAUCUUUUCUUUAUGUUUUCG | 815 |
| 180 | AACAUAAAGAAAAGA | 498 | AAAUCUUUUCUUUAUGUUUUC | 816 |
| 181 | AAAGAAAAGAUGAGC | 499 | AAUGCUCAUCUUUUCUUUAUG | 817 |
| 182 | AAGAUGGUAAAAAGA | 500 | AAUUCUUUUUACCAUCUUUGC | 818 |
| 183 | AGAUGGUAAAAAGAA | 501 | AACUUCUUUUUACCAUCUUUG | 819 |
| 184 | GGUAAAAAGAAGAAA | 502 | AAUUUUCUUCUUUUUACCAUC | 820 |
| 185 | AAAAAGAAGAAAAAG | 503 | AAUCUUUUUCUUCUUUUUACC | 821 |
| 186 | AAAAGAAGAAAAAGA | 504 | AAUUCUUUUUCUUCUUUUUAC | 822 |
| 187 | GAAAAAGAAGUCAAA | 505 | AACUUUGACUUCUUUUUCUUC | 823 |
| 188 | AAAGAAGUCAAAGAC | 506 | AAUGUCUUUGACUUCUUUUUC | 824 |
| 189 | GUCAAAGACAAAGUG | 507 | AAACACUUUGUCUUUGACUUC | 825 |
| 190 | AAAGACAAAGUGUGU | 508 | AAUACACACUUUGUCUUUGAC | 826 |
| 191 | AGACAAAGUGUGUAA | 509 | AAAUUACACACUUUGUCUUUG | 827 |
| 192 | AAAGUGUGUAAUUAU | 510 | AACAUAAUUACACACUUUGUC | 828 |
| 193 | AGUGUGUAAUUAUGU | 511 | AAUACAUAAUUACACACUUUG | 829 |
| 194 | UUUGUACUUUUUUCU | 512 | AAAAGAAAAAAGUACAAAUUG | 830 |
| 195 | CUUUUUUCUUAAGGC | 513 | AAUGCCUUAAGAAAAAAGUAC | 831 |
| 196 | UUUUCUUAAGGCAUA | 514 | AAGUAUGCCUUAAGAAAAAAG | 832 |
| 197 | AAGGCAUACUAGUAC | 515 | AAUGUACUAGUAUGCCUUAAG | 833 |
| 198 | ACUAGUACAAGUGGU | 516 | AAUACCACUUGUACUAGUAUG | 834 |
| 199 | GUACAAGUGGUAAUU | 517 | AAAAAUUACCACUUGUACUAG | 835 |
| 200 | CAAGUGGUAAUUUUU | 518 | AACAAAAAUUACCACUUGUAC | 836 |
| 201 | GUAAUUUUUGUACAU | 519 | AAAAUGUACAAAAAUUACCAC | 837 |
| 202 | AUUUUUGUACAUUAC | 520 | AAUGUAAUGUACAAAAAUUAC | 838 |
| 203 | CAUUACACUAAAUUA | 521 | AAAUAAUUUAGUGUAAUGUAC | 839 |
| 204 | UACACUAAAUUAUUA | 522 | AACUAAUAAUUUAGUGUAAUG | 840 |
| 205 | AAUUAUUAGCAUUUG | 523 | AAACAAAUGCUAAUAAUUUAG | 841 |
| 206 | UUUGUUUUAGCAUUA | 524 | AAGUAAUGCUAAAACAAAUGC | 842 |
| 207 | UUAGCAUUACCUAAU | 525 | AAAAUUAGGUAAUGCUAAAAC | 843 |
| 208 | GCUCCAUGCAGACUG | 526 | AAACAGUCUGCAUGGAGCAGG | 844 |
| 209 | CUCCAUGCAGACUGU | 527 | AAAACAGUCUGCAUGGAGCAG | 845 |
| 210 | CCAUGCAGACUGUUA | 528 | AACUAACAGUCUGCAUGGAGC | 846 |
| 211 | UGCAGACUGUUAGCU | 529 | AAAAGCUAACAGUCUGCAUGG | 847 |
| 212 | UGUUAGCUUUUACCUU | 530 | AAUAAGGUAAAAGCUAACAGUC | 848 |
| 213 | AGCUUUUACCUUAAA | 531 | AAAUUUAAGGUAAAAGCUAAC | 849 |
| 214 | UUUACCUUAAAUGCU | 532 | AAAAGCAUUUAAGGUAAAAGC | 850 |
| 215 | UUACCUUAAAUGCUU | 533 | AAUAAGCAUUUAAGGUAAAAG | 851 |
| 216 | UUUUUUUCCUCUAAG | 534 | AAACUUAGAGGAAAAAAAAAC | 852 |
| 217 | GUAUUCCCAGAGUUU | 535 | AAAAAACUCUGGGAAUACUGG | 853 |
| 218 | AGUUUUGGUUUUUGA | 536 | AAUUCAAAAACCAAAACUCUG | 854 |
| 219 | UUUUGGUUUUUGAAC | 537 | AAAGUUCAAAAACCAAAACUC | 855 |
| 220 | GCAAUGCCUGUGAAA | 538 | AAUUUUCACAGGCAUUGCUAG | 856 |
| 221 | UGAAAAAGAAACUGA | 539 | AAUUCAGUUUCUUUUUCACAG | 857 |
| 222 | AAAAAGAAACUGAAU | 540 | AAUAUUCAGUUUCUUUUUCAC | 858 |
| 223 | AAUACCUAAGAUUUC | 541 | AAAGAAAUCUUAGGUAUUCAG | 859 |
| 224 | UACCUAAGAUUUCUG | 542 | AAACAGAAAUCUUAGGUAUUC | 860 |
| 225 | UUGAUUACUUCUUAU | 543 | AAAAUAAGAAGUAAUCAACUG | 861 |
| 226 | GAUUACUUCUUAUUU | 544 | AAAAAAUAAGAAGUAAUCAAC | 862 |
| 227 | UACUUCUUAUUUUUC | 545 | AAAGAAAAAUAAGAAGUAAUC | 863 |
| 228 | AUUUUUCUUACCAAU | 546 | AAAAUUGGUAAGAAAAAUAAG | 864 |
| 229 | ACCAAUUGUGAAUGU | 547 | AAAACAUUCACAAUUGGUAAG | 865 |
| 230 | UGUUGGUGUGAAACA | 548 | AAUUGUUUCACACCAACAUUC | 866 |
| 231 | UGAAACAAAUUAAUG | 549 | AAUCAUUAAUUUGUUUCACAC | 867 |
| 232 | AUUCUGUGUUUUAUC | 550 | AAAGAUAAAACACAGAAUAGG | 868 |
| 233 | UUCUGUGUUUUAUCU | 551 | AAUAGAUAAAACACAGAAUAG | 869 |
| 234 | AAAUGGAUUAAUUAC | 552 | AAAGUAAUUAAUCCAUUUAUG | 870 |
| 235 | CUAAUUGGUUUUUAC | 553 | AAAGUAAAAACCAAUUAGAAG | 871 |
| 236 | AUUGGUUUUUACUGA | 554 | AAUUCAGUAAAAACCAAUUAG | 872 |
| 237 | UUUACUGAAACAUUG | 555 | AAUCAAUGUUUCAGUAAAAAC | 873 |
| 238 | UCAUGUCCUAUAGUU | 556 | AAAAACUAUAGGACAUGAUGC | 874 |
| 239 | CACAAAGGUUUUGUC | 557 | AAAGACAAAACCUUUGUGAAC | 875 |
| 240 | AUUUCCUUUUCACAU | 558 | AAAAUGUGAAAAGGAAAUGGC | 876 |
| 241 | UUUCCUUUUCACAUU | 559 | AAUAAUGUGAAAAGGAAAUGG | 877 |
| 242 | CAUGCAGACUGUUAG | 560 | AAGCUAACAGUCUGCAUGGAG | 878 |
| 243 | GCAGACUGUUAGCUU | 561 | AAAAAGCUAACAGUCUGCAUG | 879 |
| 244 | GACUGUUAGCUUUUA | 562 | AAGUAAAAGCUAACAGUCUGC | 880 |
| 245 | UUAGCUUUUACCUUA | 563 | AAUUAAGGUAAAAGCUAACAG | 881 |
| 246 | UAAAUGCUUAUUUUA | 564 | AAUUAAAAUAAGCAUUUAAGG | 882 |
| 247 | AGUGGAAGUUUUUUU | 565 | AAAAAAAAAACUUCCACUGUC | 883 |
| 248 | GUGGAAGUUUUUUUU | 566 | AAAAAAAAAAACUUCCACUGU | 884 |
| 249 | CUAAGUGCCAGUAUU | 567 | AAGAAUACUGGCACUUAGAGG | 885 |
| 250 | AGUGCCAGUAUUCCC | 568 | AAUGGGAAUACUGGCACUUAG | 886 |
| 251 | CCAGUAUUCCCAGAG | 569 | AAACUCUGGGAAUACUGGCAC | 887 |
| 252 | AGUAUUCCCAGAGUU | 570 | AAAAACUCUGGGAAUACUGGC | 888 |
| 253 | GUAUUCCCAGAGUUU | 571 | AAAAAACUCUGGGAAUACUGG | 889 |
| 254 | UAUUCCCAGAGUUUU | 572 | AACAAAACUCUGGGAAUACUG | 890 |
| 255 | UUCCCAGAGUUUUGG | 573 | AAACCAAAACUCUGGGAAUAC | 891 |
| 256 | AGAGUUUUGGUUUUU | 574 | AACAAAAACCAAAACUCUGGG | 892 |
| 257 | GAGUUUUGGUUUUUG | 575 | AAUCAAAAACCAAAACUCUGG | 893 |
| 258 | GUUUUGGUUUUUGAA | 576 | AAGUUCAAAAACCAAAACUCU | 894 |
| 259 | UUGGUUUUUGAACUA | 577 | AACUAGUUCAAAAACCAAAAC | 895 |
| 260 | UUUUGAACUAGCAAU | 578 | AACAUUGCUAGUUCAAAAACC | 896 |
| 261 | CUAGCAAUGCCUGUG | 579 | AAUCACAGGCAUUGCUAGUUC | 897 |
| 262 | AUGCCUGUGAAAAAG | 580 | AAUCUUUUUCACAGGCAUUGC | 898 |
| 263 | UGCCUGUGAAAAAGA | 581 | AAUUCUUUUUCACAGGCAUUG | 899 |
| 264 | UGUGAAAAAGAAACU | 582 | AACAGUUUCUUUUUCACAGGC | 900 |
| 265 | GUGAAAAAGAAACUG | 583 | AAUCAGUUUCUUUUUCACAGG | 901 |
| 266 | AAAAGAAACUGAAUA | 584 | AAGUAUUCAGUUUCUUUUUCA | 902 |
| 267 | ACUGAAUACCUAAGA | 585 | AAAUCUUAGGUAUUCAGUUUC | 903 |
| 268 | UCUUGGGGUUUUUGG | 586 | AAACCAAAAACCCCAAGACAG | 904 |
| 269 | UUGGGGUUUUUGGUG | 587 | AAGCACCAAAAACCCCAAGAC | 905 |
| 270 | GGGGUUUUUGGUGCA | 588 | AAAUGCACCAAAAACCCCAAG | 906 |
| 271 | UGCAGUGUUUUUGGU | 589 | AACACCAAAAACACUGCAUGC | 907 |
| 272 | UUUGGUGCAUGCAGU | 590 | AAAACUGCAUGCACCAAAAAC | 908 |
| 273 | UUUCACAUUAGAUAA | 591 | AAUUUAUCUAAUGUGAAAAGG | 909 |
| 274 | UUCACAUUAGAUAAA | 592 | AAAUUUAUCUAAUGUGAAAAG | 910 |
| 275 | UGAAAUGGGGAUUAU | 593 | AAAAUAAUCCCCAUUUCAUAC | 911 |
| 276 | UUUUGGGGCUAUAUU | 594 | AAAAAUAUAGCCCCAAAAUGG | 912 |
| 277 | UUUGGGGCUAUAUUU | 595 | AAUAAAUAUAGCCCCAAAAUG | 913 |
| 278 | AAGAUUUUAACAAGU | 596 | AAUACUUGUUAAAAUCUUUUC | 914 |
| 279 | GUAUAAAAAAUUCUC | 597 | AAUGAGAAUUUUUUAUACUUG | 915 |
| 280 | AGGAAUUAAAUGUAG | 598 | AAACUACAUUUAAUUCCUAUG | 916 |
| 281 | CUUUCAUAGUAUAAC | 599 | AAAGUUAUACUAUGAAAGAGC | 917 |
| 282 | UUUCAUAGUAUAACU | 600 | AAAAGUUAUACUAUGAAAGAG | 918 |
| 283 | UCAUAGUAUAACUUU | 601 | AAUAAAGUUAUACUAUGAAAG | 919 |
| 284 | UAAAUCUUUUCUUCA | 602 | AAUUGAAGAAAAGAUUUAAAG | 920 |
| 285 | CAACUUGAGUCUUUG | 603 | AAUCAAAGACUCAAGUUGAAG | 921 |
| 286 | CUUGAGUCUUUGAAG | 604 | AAUCUUCAAAGACUCAAGUUG | 922 |
| 287 | UCUUUGAAGAUAGUU | 605 | AAAAACUAUCUUCAAAGACUC | 923 |
| 288 | UUUGAAGAUAGUUUU | 606 | AAUAAAACUAUCUUCAAAGAC | 924 |
| 289 | UGAAGAUAGUUUUAA | 607 | AAAUUAAAACUAUCUUCAAAG | 925 |
| 290 | UUAUAGCUUAUUAGG | 608 | AAACCUAAUAAGCUAUAACUG | 926 |
| 291 | UAUUAGGUGUUGAAG | 609 | AAUCUUCAACACCUAAUAAGC | 927 |
| 292 | UUCAUAGAGAGUUUC | 610 | AAUGAAACUCUCUAUGAAAGC | 928 |
| 293 | GCAUUGGUUAGUCAA | 611 | AAUUUGACUAACCAAUGCAUG | 929 |
| 294 | UGCUUUUGUUUCUUA | 612 | AAUUAAGAAACAAAAGCAAUG | 930 |
| 295 | UUUGUUUCUUAAGAA | 613 | AAUUUCUUAAGAAACAAAAGC | 931 |
| 296 | UUGUUUCUUAAGAAA | 614 | AAUUUUCUUAAGAAACAAAAG | 932 |
| 297 | AAGAAAACAAACUCU | 615 | AAAAGAGUUUGUUUUCUUAAG | 933 |
| 298 | AACAAACUCUUUUUU | 616 | AAUAAAAAAGAGUUUGUUUUC | 934 |
| 299 | UGAAGUGAAAAAGUU | 617 | AAAAACUUUUUCACUUCAUUG | 935 |
| 300 | GUGAAAAAGUUUUAC | 618 | AAUGUAAAACUUUUUCACUUC | 936 |
| 301 | UUAACACUGGUUAAA | 619 | AAAUUUAACCAGUGUUAAGAG | 937 |
| 302 | AACACUGGUUAAAUU | 620 | AAUAAUUUAACCAGUGUUAAG | 938 |
| 303 | AAAUUAACAUUGCAU | 621 | AAUAUGCAAUGUUAAUUUAAC | 939 |
| 304 | UAAACACUUUUCAAG | 622 | AAACUUGAAAAGUGUUUAUGC | 940 |
| 305 | UCCUUUUGAUAAAUU | 623 | AAAAAUUUAUCAAAAGGAUUG | 941 |
| 306 | ACUUAGGUUCUAGAU | 624 | AAUAUCUAGAACCUAAGUCAC | 942 |
| 307 | UUAGGACUCUGAUUU | 625 | AAAAAAUCAGAGUCCUAAAAG | 943 |
| 308 | CACUUACUAUCCAUU | 626 | AAAAAUGGAUAGUAAGUGAUG | 944 |
| 309 | UUACUAUCCAUUUCU | 627 | AAAAGAAAUGGAUAGUAAGUG | 945 |
| 310 | ACUAUCCAUUUCUUC | 628 | AAUGAAGAAAUGGAUAGUAAG | 946 |
| 311 | UCCAUUUCUUCAUGU | 629 | AAAACAUGAAGAAAUGGAUAG | 947 |
| 312 | UUUCUUCAUGUUAAA | 630 | AAUUUUAACAUGAAGAAAUGG | 948 |
| 313 | GUCAUCUCAAACUCU | 631 | AAAAGAGUUUGAGAUGACUUC | 949 |
| 314 | CUCAAACUCUUAGUU | 632 | AAAAACUAAGAGUUUGAGAUG | 950 |
| 315 | AAACUCUUAGUUUUU | 633 | AAAAAAAACUAAGAGUUUGAG | 951 |
| 316 | UGUAAUUUAUAUUCC | 634 | AAUGGAAUAUAAAUUACAUAG | 952 |
| 317 | AAGGAUACACUUAUU | 635 | AAAAAUAAGUGUAUCCUUAUG | 953 |
| 318 | CAAUCUGUAAAUUUU | 636 | AAAAAAAUUUACAGAUUGUGC | 954 |
| 319 | UGUUACACCAUCUUC | 637 | AAUGAAGAUGGUGUAACAUAG | 955 |

In some embodiments, the RNA duplex molecule (aiRNA) comprises a sense strand sequence selected from the group consisting of SEQ ID NOs: 320-637. In some embodiments, the RNA duplex molecule (aiRNA) comprises an antisense strand sequence selected from the group consisting of SEQ ID NOs: 638-955. In some embodiments, the RNA duplex molecule (aiRNA) comprises a sense strand sequence selected from the group consisting of SEQ ID NOs: 320-637 and an antisense strand sequence selected from the group consisting of SEQ ID NOs: 638-955.

In some embodiments, the RNA duplex molecule (aiRNA) comprises a sense strand sequence that is at least, e.g., 50%, 60%, 70%, 75%, 80%, 85%, 90%, 95% or more identical to a sequence selected from the group consisting of SEQ ID NOs: 320-637. In some embodiments, the RNA duplex molecule (aiRNA) comprises an antisense strand sequence that is at least, e.g., 50%, 60%, 70%, 75%, 80%, 85%, 90%, 95% or more identical to a sequence selected from the group consisting of SEQ ID NOs: 638-955. In some embodiments, the RNA duplex molecule (aiRNA) comprises a sense strand sequence that is at least, e.g., 50%, 60%, 70%, 75%, 80%, 85%, 90%, 95% or more identical to a sequence selected from the group consisting of SEQ ID NOs: 320-637 and an antisense strand sequence that is at least, e.g., 50%, 60%, 70%, 75%, 80%, 85%, 90%, 95% or more identical to a sequence selected from the group consisting of SEQ ID NOs: 638-955.

In some embodiments, at least one nucleotide of the sequence of 5′ overhang is selected from the group consisting of A, U, and dT.

In some embodiments, the GC content of the double stranded region is 20%-70%.

In some embodiments, the first strand has a length from 19-22 nucleotides.

In some embodiments, the first strand has a length of 21 nucleotides. In a further embodiment, the second strand has a length of 14-16 nucleotides.

In some embodiments, the first strand has a length of 21 nucleotides, and the second strand has a length of 15 nucleotides. In a further embodiment, the first strand has a 3′-overhang of 2-4 nucleotides. In an even further embodiment, the first strand has a 3′-overhang of 3 nucleotides.

In some embodiments, the duplex RNA molecule contains at least one modified nucleotide or its analogue. In a further embodiment, the at least one modified nucleotide or its analogue is sugar-, backbone-, and/or base-modified ribonucleotide. In an even further embodiment, the backbone-modified ribonucleotide has a modification in a phosphodiester linkage with another ribonucleotide. In some embodiments, the phosphodiester linkage is modified to include at least one of a nitrogen or a sulphur heteroatom. In another embodiment, the nucleotide analogue is a backbone-modified ribonucleotide containing a phosphothioate group.

In some embodiments, the at least one modified nucleotide or its analogue is an unusual base or a modified base. In another embodiment, the at least one modified nucleotide or its analogue comprises inosine, or a tritylated base.

In a further embodiment, the nucleotide analogue is a sugar-modified ribonucleotide, wherein the 2′-OH group is replaced by a group selected from H, OR, R, halo, SH, SR, NH₂, NHR, NR₂, or CN, wherein each R is independently C1-C6 alkyl, alkenyl or alkynyl, and halo is F, Cl, Br or I.

In some embodiments, the first strand comprises at least one deoxynucleotide. In a further embodiment, the at least one deoxynucleotides are in one or more regions selected from the group consisting of 3′-overhang, 5′-overhang, and double-stranded region. In another embodiment, the second strand comprises at least one deoxynucleotide.

The present invention also provides a method of modulating K-Ras expression, e.g., silencing K-Ras expression or otherwise reducing K-Ras expression, in a cell or an organism comprising the steps of contacting said cell or organism with an asymmetrical duplex RNA molecule of the disclosure under conditions wherein selective K-Ras gene silencing can occur, and mediating a selective K-Ras gene silencing effected by the duplex RNA molecule towards K-Ras or nucleic acid having a sequence portion substantially corresponding to the double-stranded RNA. In a further embodiment, said contacting step comprises the step of introducing said duplex RNA molecule into a target cell in culture or in an organism in which the selective K-Ras silencing can occur. In an even further embodiment, the introducing step is selected from the group consisting of transfection, lipofection, electroporation, infection, injection, oral administration, inhalation, topical and regional administration. In another embodiment, the introducing step comprises using a pharmaceutically acceptable excipient, carrier, or diluent selected from the group consisting of a pharmaceutical carrier, a positive-charge carrier, a liposome, a protein carrier, a polymer, a nanoparticle, a nanoemulsion, a lipid, and a lipoid.

In some embodiments, the modulating method is used for determining the function or utility of a gene in a cell or an organism.

In some embodiments, the modulating method is used for treating or preventing a disease or an undesirable condition. In some embodiments, the disease or undesirable condition is a cancer, for example, gastric cancer.

The disclosure provides compositions and methods for targeting K-Ras in the treatment, prevention, delaying the progression of, or otherwise ameliorating a symptom of gastric cancer. In some embodiments, the method comprises administering to subject in need thereof a therapeutically effective amount of a duplex RNA molecule of the disclosure. In some embodiments, the subject is human. In some embodiments, the subject is suffering from gastric cancer. In some embodiments, the subject is diagnosed with gastric cancer. In some embodiments, the subject is predisposed to gastric cancer.

The disclosure also provides compositions and methods for targeting K-Ras to inhibit the survival and/or proliferation of cancer stem cells. In some embodiments, the method comprises administering to subject in need thereof a therapeutically effective amount of a duplex RNA molecule of the disclosure. In some embodiments, the subject is human. In some embodiments, the subject is suffering from gastric cancer. In some embodiments, the subject is diagnosed with gastric cancer. In some embodiments, the subject is predisposed to gastric cancer.

The disclosure also provides compositions and methods for targeting K-Ras in the inhibition of to inhibit the survival and/or proliferation of CSCs in the treatment, prevention, delaying the progression of, or otherwise ameliorating a symptom of gastric cancer. In some embodiments, the method comprises administering to subject in need thereof a therapeutically effective amount of a duplex RNA molecule of the disclosure. In some embodiments, the subject is human. In some embodiments, the subject is suffering from gastric cancer. In some embodiments, the subject is diagnosed with gastric cancer. In some embodiments, the subject is predisposed to gastric cancer.

The disclosure also provides a method for treating cancer in a selected patient population, the method comprising the steps of: (a) measuring a level of mutant K-Ras gene amplification in a biological sample obtained from a patient candidate diagnosed of a cancer; (b) confirming that the patient candidate's mutant K-Ras gene amplification level is above a benchmark level; and (c) administering to the patient candidate a duplex RNA molecule comprising a first strand comprising a nucleotide sequence with a length from 18-23 nucleotides, wherein the nucleotide sequence of the first strand is substantially complementary to a target K-Ras mRNA sequence, and a second strand comprising a nucleotide sequence with a length from 12-17 nucleotides, wherein the second strand is substantially complementary to the first strand, and forms a double-stranded region with the first strand, wherein the first strand has a 3′-overhang from 1-9 nucleotides, and a 5′-overhang from 0-8 nucleotides, and wherein said duplex RNA molecule is capable of effecting selective K-Ras gene silencing.

In some embodiments, the steps (a), (b), and (c) may be performed by one actor or several actors.

In some embodiments, a patient candidate's mutant K-Ras gene amplification level is considered to be above a benchmark level if it is at least, e.g., 2-fold greater relative to that of a control patient who would not respond favorably to the claimed treatment method according to the present invention. Likewise, a skilled physician may determine that the optimal benchmark level of the DNA copy number is, e.g., about 3-fold or 4-fold greater relative to that of a non-responsive patient, based on the data presented in the present disclosure.

The disclosure also provides a method for treating cancer in a selected patient population, the method comprising the steps of: (a) measuring an expression level of mutant K-Ras protein in a biological sample obtained from a patient candidate diagnosed of a cancer; (b) confirming that the patient candidate's mutant K-Ras protein expression level is above a benchmark level; and (c) administering to the patient candidate a duplex RNA molecule comprising a first strand comprising a nucleotide sequence with a length from 18-23 nucleotides, wherein the nucleotide sequence of the first strand is substantially complementary to a target K-Ras mRNA sequence, and a second strand comprising a nucleotide sequence with a length from 12-17 nucleotides, wherein the second strand is substantially complementary to the first strand, and forms a double-stranded region with the first strand, wherein the first strand has a 3′-overhang from 1-9 nucleotides, and a 5′-overhang from 0-8 nucleotides, and wherein said duplex RNA molecule is capable of effecting selective K-Ras gene silencing.

In some embodiments, the steps (a), (b), and (c) may be performed by one actor or several actors.

In some embodiments, a patient candidate's mutant K-Ras protein expression level is considered to be above a benchmark level if it is at least, e.g., 2-fold greater relative to that of a control patient who would not respond favorably to the claimed treatment method according to the present invention. Likewise, a skilled physician may determine that the optimal benchmark level of the mutant K-Ras protein expression is, e.g., about 3-fold or 4-fold greater relative to that of a non-responsive patient, based on the data presented in the present disclosure.

The present invention further provides a kit. The kit comprises a first RNA strand with a length from 18-23 nucleotides and a second RNA strand with a length from 12-17 nucleotides, wherein the second strand is substantially complementary to the first strand, and capable of forming a duplex RNA molecule with the first strand, wherein the duplex RNA molecule has a 3′-overhang from 1-9 nucleotides, and a 5′-overhang from 0-8 nucleotides, wherein said duplex RNA molecule is capable of effecting K-Ras specific gene silencing.

The present invention also provides a method of preparing the duplex RNA molecule. The method comprises the steps of synthesizing the first strand and the second strand, and combining the synthesized strands under conditions, wherein the duplex RNA molecule is formed, which is capable of effecting sequence-specific gene silencing. In some embodiments, the method further comprises a step of introducing at least one modified nucleotide or its analogue into the duplex RNA molecule during the synthesizing step, after the synthesizing and before the combining step, or after the combining step. In another embodiment, the RNA strands are chemically synthesized, or biologically synthesized.

The present invention provides an expression vector. The vector comprises a nucleic acid or nucleic acids encoding the duplex RNA molecule operably linked to at least one expression-control sequence. In some embodiments, the vector comprises a first nucleic acid encoding the first strand operably linked to a first expression-control sequence, and a second nucleic acid encoding the second strand operably linked to a second expression-control sequence. In another embodiment, the vector is a viral, eukaryotic, or bacterial expression vector.

The present invention also provides a cell. In some embodiments, the cell comprises the vector. In another embodiment, the cell comprises the duplex RNA molecule. In a further embodiment, the cell is a mammalian, avian, or bacterial cell.

The modulating method can also be used for studying drug target in vitro or in vivo. The present invention provides a reagent comprising the duplex RNA molecule.

The present invention also provides a method of preparing a duplex RNA molecule of the disclosure comprising the steps of synthesizing the first strand and the second strand, and combining the synthesized strands under conditions, wherein the duplex RNA molecule is formed, which is capable of effecting K-Ras sequence-specific gene silencing. In some embodiments, the RNA strands are chemically synthesized, or biologically synthesized. In another embodiment, the first strand and the second strand are synthesized separately or simultaneously.

In some embodiments, the method further comprises a step of introducing at least one modified nucleotide or its analogue into the duplex RNA molecule during the synthesizing step, after the synthesizing and before the combining step, or after the combining step.

The present invention further provides a pharmaceutical composition. The pharmaceutical composition comprises as an active agent at least one duplex RNA molecule and one or more carriers selected from the group consisting of a pharmaceutical carrier, a positive-charge carrier, a liposome, a protein carrier, a polymer, a nanoparticle, a cholesterol, a lipid, and a lipoid.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1(A) shows an in vitro study in which aiRNA ID NO: 21 (“aiK-Ras #1”) was used to target K-Ras Target SEQ ID NO: 22 to determine the IC₅₀ for aiK-Ras #1.

FIG. 1(B) shows an in vitro study in which aiRNA ID NO: 142 (“aiK-Ras #2”) was used to target K-Ras Target SEQ ID NO: 142 to determine the IC₅₀ for aiK-Ras #2.

FIG. 2(A) shows detection of siRNA and aiRNA loading to RISC by northern blot analysis.

FIG. 2(B) shows detection of TLR3/aiRNA or siRNA binding.

FIG. 2(C) shows that TLR3/RNA complexes were immunoprecipitated with anti-HA antibody.

FIG. 3(A) shows colony formation assay in AGS and DLD1 transfected with aiK-Ras #1 or aiK-Ras #2.

FIG. 3(B) shows western blot analysis of lysate from AGS and DLD1.

FIG. 3(C) shows colony formation assay results in large cell panel.

FIG. 4 shows western blot analysis of K-Ras and EGFR-RAS pathway molecules.

FIG. 5(A) shows that aiK-Ras sensitivity was correlated with K-Ras amplification in K-Ras mutant large cell panel.

FIG. 5(B) shows that aiK-Ras sensitivity was correlated with K-Ras amplification in K-Ras mutant large cell panel.

FIG. 6(A) shows stemness gene expression in CSC culture.

FIG. 6(B) shows the results of sphere formation assay in various cell lines.

FIG. 6(C) shows depletion of CD44-high population in AGS and DLD1 cells with aiK-Ras #1 and aiK-Ras #2.

FIG. 7(A) shows heat map of CSC-related genes in cancer cells transfected with aiK-Ras.

FIG. 7(B) shows confirmation of down-regulated Notch signaling by western blot.

### DETAILED DESCRIPTION OF THE INVENTION

The present invention relates to asymmetric duplex RNA molecules that are capable of effecting selective K-Ras gene silencing in a eukaryotic cell. In some embodiments, the duplex RNA molecule comprises a first strand and a second strand. The first strand is longer than the second strand. The second strand is substantially complementary to the first strand, and forms a double-stranded region with the first strand.

The protein K-Ras is a molecular switch that under normal conditions regulates cell growth and cell division. Mutations in this protein lead to the formation of tumors through continuous cell growth. About 30% of human cancers have a mutated Ras protein that is constitutively bound to GTP due to decreased GTPase activity and insensitivity to GAP action. Ras is also an important factor in many cancers in which it is not mutated but rather functionally activated through inappropriate activity of other signal transduction elements. Mutated K-Ras proteins are found in a large proportion of all tumor cells. K-Ras protein occupies a central position of interest. The identification of oncogenically mutated K-Ras in many human cancers led to major efforts to target this constitutively activated protein as a rational and selective treatment. Despite decades of active agent research, significant challenges still remain to develop therapeutic inhibitors of K-Ras.

The compositions and methods provided herein are useful in elucidating the function of K-Ras in the cancer development and maintenance. The compositions and methods use asymmetric interfering RNAs (aiRNAs) that are able to silence target genes with high potency leading to long-lasting knockdown, and reducing off-target effects, and investigated the dependency of K-Ras on cell survival in several types of human cancer cell lines. Much to our surprise, we found K-Ras plays a more significant role for gastric cancer maintenance compared to other types of cancer aiRNA-induced silencing of K-Ras was found to inhibit the cell proliferation of gastric cancer cells and the ability of gastric cancer cells to form colonies compared to other cancer types. Accumulating evidence has revealed that cancer stem cells (CSCs) are highly associated with prognosis, metastasis, and recurrence. To investigate the effect of K-Ras on CSCs, we tested the K-Ras gene silencing effects on an in vitro CSC culturing system. As a result, K-Ras inhibition decreased the colonies derived from gastric CSCs and altered the gene expression patterns of several genes involved in “stemness” compared to other cancer types. The results of these studies suggest that gastric cancer and gastric CSCs are affected by the K-Ras oncogene and that Kras aiRNAs are promising therapeutic candidates for the treatment of gastric cancer. Accordingly, the disclosure provides compositions and methods for targeting K-Ras in the treatment, prevention, delaying the progression of, or otherwise ameliorating a symptom of gastric cancer. The disclosure also provides compositions and methods for targeting K-Ras to inhibit the survival and/or proliferation of CSCs, as well as compositions and methods for targeting K-Ras in the inhibition of to inhibit the survival and/or proliferation of CSCs in the treatment, prevention, delaying the progression of, or otherwise ameliorating a symptom of gastric cancer. In some embodiments, the method comprises administering to subject in need thereof a therapeutically effective amount of a duplex RNA molecule of the disclosure. In some embodiments, the subject is human. In some embodiments, the subject is suffering from gastric cancer. In some embodiments, the subject is diagnosed with gastric cancer. In some embodiments, the subject is predisposed to gastric cancer.

In some embodiments, the duplex RNA molecule used in the compositions and methods of the disclosure has a 3′-overhang from 1-8 nucleotides and a 5′-overhang from 1-8 nucleotides, a 3′-overhang from 1-10 nucleotides and a blunt end, or a 5′-overhang from 1-10 nucleotides and a blunt end. In another embodiment, the duplex RNA molecule has two 5′-overhangs from 1-8 nucleotides or two 3′-overhangs from 1-10 nucleotides. In a further embodiment, the first strand has a 3′-overhang from 1-8 nucleotides and a 5′-overhang from 1-8 nucleotides. In an even further embodiment, the duplex RNA molecule is an isolated duplex RNA molecule.

In some embodiments, the first strand has a 3′-overhang from 1-10 nucleotides, and a 5′-overhang from 1-10 nucleotides or a 5′-blunt end. In another embodiment, the first strand has a 3¹-overhang from 1-10 nucleotides, and a 5¹-overhang from 1-10 nucleotides. In an alternative embodiment, the first strand has a 3′-overhang from 1-10 nucleotides, and a 5′-blunt end.

In some embodiments, the first strand has a length from 5-100 nucleotides, from 12-30 nucleotides, from 15-28 nucleotides, from 18-27 nucleotides, from 19-23 nucleotides, from 20-22 nucleotides, or 21 nucleotides.

In another embodiment, the second strand has a length from 3-30 nucleotides, from 12-26 nucleotides, from 13-20 nucleotides, from 14-23 nucleotides, 14 or 15 nucleotides.

In some embodiments, the first strand has a length from 5-100 nucleotides, and the second strand has a length from 3-30 nucleotides; or the first strand has a length from 10-30 nucleotides, and the second strand has a length from 3-29 nucleotides; or the first strand has a length from 12-30 nucleotides and the second strand has a length from 10-26 nucleotides; or the first strand has a length from 15-28 nucleotides and the second strand has a length from 12-26 nucleotides; or the first strand has a length from 19-27 nucleotides and the second strand has a length from 14-23 nucleotides; or the first strand has a length from 20-22 nucleotides and the second strand has a length from 14-15 nucleotides. In a further embodiment, the first strand has a length of 21 nucleotides and the second strand has a length of 13-20 nucleotides, 14-19 nucleotides, 14-17 nucleotides, 14 or 15 nucleotides.

In some embodiments, the first strand is at least 1, 2, 3, 4, 5, 6, 7, 8, 9, or 10 nucleotides longer than the second strand.

In some embodiments, the duplex RNA molecule further comprises 1-10 unmatched or mismatched nucleotides. In a further embodiment, the unmatched or mismatched nucleotides are at or near the 3′ recessed end. In an alternative embodiment, the unmatched or mismatched nucleotides are at or near the 5′ recessed end. In an alternative embodiment, the unmatched or mismatched nucleotides are at the double-stranded region. In another embodiment, the unmatched or mismatched nucleotide sequence has a length from 1-5 nucleotides. In an even further embodiment, the unmatched or mismatched nucleotides form a loop structure.

In some embodiments, the first strand or the second strand contains at least one nick, or formed by two nucleotide fragments.

In some embodiments, the gene silencing is achieved through one or two, or all of RNA interference, modulation of translation, and DNA epigenetic modulations.

In some embodiments, the target K-Ras mRNA sequence to be silenced is a target sequence shown in Table 1.

In some embodiments, the RNA duplex molecule, also referred to herein as an asymmetrical interfering RNA molecule or aiRNA molecule, comprises a sense strand sequence, an antisense strand sequence or a combination of a sense strand sequence and antisense strand sequence selected from those shown in Table 2.

In some embodiments, the RNA duplex molecule (aiRNA) comprises a sense strand sequence selected from the group consisting of SEQ ID NOs: 320-637. In some embodiments, the RNA duplex molecule (aiRNA) comprises an antisense strand sequence selected from the group consisting of SEQ ID NOs: 638-955. In some embodiments, the RNA duplex molecule (aiRNA) comprises a sense strand sequence selected from the group consisting of SEQ ID NOs: 320-637 and an antisense strand sequence selected from the group consisting of SEQ ID NOs: 638-955.

In some embodiments, the RNA duplex molecule (aiRNA) comprises a sense strand sequence that is at least, e.g, 50%, 60%, 70%, 75%, 80%, 85%, 90%, 95% or more identical to a sequence selected from the group consisting of SEQ ID NOs: 320-637. In some embodiments, the RNA duplex molecule (aiRNA) comprises an antisense strand sequence that is at least, e.g., 50%, 60%, 70%, 75%, 80%, 85%, 90%, 95% or more identical to a sequence selected from the group consisting of SEQ ID NOs: 638-955. In some embodiments, the RNA duplex molecule (aiRNA) comprises a sense strand sequence that is at least, e.g., 50%, 60%, 70%, 75%, 80%, 85%, 90%, 95% or more identical to a sequence selected from the group consisting of SEQ ID NOs: 320-637 and an antisense strand sequence that is at least, e.g., 50%, 60%, 70%, 75%, 80%, 85%, 90%, 95% or more identical to a sequence selected from the group consisting of SEQ ID NOs: 638-955.

As used in the specification and claims, the singular form “a”, “an”, and “the” include plural references unless the context clearly dictate otherwise. For example, the term “a cell” includes a plurality of cells including mixtures thereof.

As used herein, a “double stranded RNA,” a “duplex RNA” or a “RNA duplex” refers to an RNA of two strands and with at least one double-stranded region, and includes RNA molecules that have at least one gap, nick, bulge, and/or bubble either within a double-stranded region or between two neighboring double-stranded regions. If one strand has a gap or a single-stranded region of unmatched nucleotides between two double-stranded regions, that strand is considered as having multiple fragments. A double-stranded RNA as used here can have terminal overhangs on either end or both ends. In some embodiments, the two strands of the duplex RNA can be linked through certain chemical linker.

As used herein, an “antisense strand” refers to an RNA strand that has substantial sequence complementarity against a target messenger RNA.

The term “isolated” or “purified” as used herein refers to a material that is substantially or essentially free from components that normally accompany it in its native state. Purity and homogeneity are typically determined using analytical chemistry techniques such as polyacrylamide gel electrophoresis or high performance liquid chromatography.

As used herein, “modulating” and its grammatical equivalents refer to either increasing or decreasing (e.g., silencing), in other words, either up-regulating or down-regulating. As used herein, “gene silencing” refers to reduction of gene expression, and may refer to a reduction of gene expression about, e.g., 20%, 30%, 40%, 50%, 60%, 70%, 80%, 90% or 95% of the targeted gene.

As used herein, the term “subject” refers to any animal (e.g., a mammal), including, but not limited to humans, non-human primates, rodents, and the like, which is to be the recipient of a particular treatment. Under some circumstances, the terms “subject” and “patient” are used interchangeably herein in reference to a human subject.

Terms such as “treating” or “treatment” or “to treat” or “alleviating” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

As used herein, the terms “inhibiting”, “to inhibit” and their grammatical equivalents, when used in the context of a bioactivity, refer to a down-regulation of the bioactivity, which may reduce or eliminate the targeted function, such as the production of a protein or the phosphorylation of a molecule. When used in the context of an organism (including a cell), the terms refer to a down-regulation of a bioactivity of the organism, which may reduce or eliminate a targeted function, such as the production of a protein or the phosphorylation of a molecule. In particular embodiments, inhibition may refer to a reduction of about, e.g., 20%, 30%, 40%, 50%, 60%, 70%, 80%, 90% or 95% of the targeted activity. When used in the context of a disorder or disease, the terms refer to success at preventing the onset of symptoms, alleviating symptoms, or eliminating the disease, condition or disorder.

As used herein, the term “substantially complementary” refers to complementarity in a base-paired, double-stranded region between two nucleic acids and not any single-stranded region such as a terminal overhang or a gap region between two double-stranded regions. The complementarity does not need to be perfect; there may be any number of base pair mismatches, for example, between the two nucleic acids. However, if the number of mismatches is so great that no hybridization can occur under even the least stringent hybridization conditions, the sequence is not a substantially complementary sequence. When two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, substantially complementary sequences can refer to sequences with base-pair complementarity of, e.g., 100%, 95%, 90%, 80%, 75%, 70%, 60%, 50% or less, or any number in between, in a double-stranded region.

RNA interference (abbreviated as RNAi) is a cellular process for the targeted destruction of single-stranded RNA (ssRNA) induced by double-stranded RNA (dsRNA). The ssRNA is gene transcript such as a messenger RNA (mRNA). RNAi is a form of post-transcriptional gene silencing in which the dsRNA can specifically interfere with the expression of genes with sequences that are complementary to the dsRNA. The antisense RNA strand of the dsRNA targets a complementary gene transcript such as a messenger RNA (mRNA) for cleavage by a ribonuclease.

In RNAi process, long dsRNA is processed by a ribonuclease protein Dicer to short forms called small interfering RNA (siRNA). The siRNA is separated into guide (or antisense) strand and passenger (or sense) strand. The guide strand is integrated into RNA-induced-silencing-complex (RISC), which is a ribonuclease-containing multi-protein complex. The complex then specifically targets complementary gene transcripts for destruction.

RNAi has been shown to be a common cellular process in many eukaryotes. RISC, as well as Dicer, is conserved across the eukaryotic domain. RNAi is believed to play a role in the immune response to virus and other foreign genetic material.

Small interfering RNAs (siRNAs) are a class of short double-stranded RNA (dsRNA) molecules that play a variety of roles in biology. Most notably, it is involved in the RNA interference (RNAi) pathway where the siRNA interferes with the expression of a specific gene. In addition, siRNAs also play roles in the processes such as an antiviral mechanism or shaping the chromatin structure of a genome. In some embodiments, siRNA has a short (19-21 nt) double-strand RNA (dsRNA) region with 2-3 nucleotide 3′ overhangs with 5′-phosphate and 3′-hydroxyl termini.

Dicer is a member of RNase III ribonuclease family. Dicer cleaves long, double-stranded RNA (dsRNA), pre-microRNA (miRNA), and short hairpin RNA (shRNA) into short double-stranded RNA fragments called small interfering RNA (siRNA) about 20-25 nucleotides long, usually with a two-base overhang on the 3′ end. Dicer catalyzes the first step in the RNA interference pathway and initiates formation of the RNA-induced silencing complex (RISC), whose catalytic component argonaute is an endonuclease capable of degrading messenger RNA (mRNA) whose sequence is complementary to that of the siRNA guide strand.

As used herein, an effective siRNA sequence is a siRNA that is effective in triggering RNAi to degrade the transcripts of a target gene. Not every siRNA complementary to the target gene is effective in triggering RNAi to degrade the transcripts of the gene. Indeed, time-consuming screening is usually necessary to identify an effective siRNA sequence. In some embodiments, the effective siRNA sequence is capable of reducing the expression of the target gene by more than 90%, more than 80%, more than 70%, more than 60%, more than 50%, more than 40%, or more than 30%.

The present invention uses a structural scaffold called asymmetric interfering RNA (aiRNA) that can be used to effect siRNA-like results, and also to modulate miRNA pathway activities, initially described in detail PCT Publications WO 2009/029688 and WO 2009/029690, the contents of which are hereby incorporated by reference in their entirety.

The structural design of aiRNA is not only functionally potent in effecting gene regulation, but also offers several advantages over the current state-of-art, RNAi regulators (mainly antisense, siRNA). Among the advantages, aiRNA can have RNA duplex structure of much shorter length than the current siRNA constructs, which should reduce the cost of synthesis and abrogate or reduce length-dependent triggering of nonspecific interferon-like immune responses from host cells. The shorter length of the passenger strand in aiRNA should also eliminate or reduce the passenger strand's unintended incorporation in RISC, and in turn, reduce off-target effects observed in miRNA-mediated gene silencing. AiRNA can be used in all areas that current miRNA-based technologies are being applied or contemplated to be applied, including biology research, R&D in biotechnology and pharmaceutical industries, and miRNA-based diagnostics and therapies.

In some embodiments, the first strand comprises a sequence being substantially complimentary to a target K-Ras mRNA sequence. In another embodiment, the second strand comprises a sequence being substantially complimentary to a target K-Ras mRNA sequence.

The present invention is pertinent to asymmetrical double stranded RNA molecules that are capable of effecting K-Ras gene silencing. In some embodiments, an RNA molecule of the present invention comprises a first strand and a second strand, wherein the second strand is substantially complementary, or partially complementary to the first strand, and the first strand and the second strand form at least one double-stranded region, wherein the first strand is longer than the second strand (length asymmetry). The RNA molecule of the present invention has at least one double-stranded region, and two ends independently selected from the group consisting of a 5′-overhang, a 3′-overhang, and a blunt.

Any single-stranded region of the RNA molecule of the invention, including any terminal overhangs and gaps in between two double-stranded regions, can be stabilized against degradation, either through chemical modification or secondary structure. The RNA strands can have unmatched or imperfectly matched nucleotides. Each strand may have one or more nicks (a cut in the nucleic acid backbone), gaps (a fragmented strand with one or more missing nucleotides), and modified nucleotides or nucleotide analogues. Not only can any or all of the nucleotides in the RNA molecule chemically modified, each strand may be conjugated with one or more moieties to enhance its functionality, for example, with moieties such as one or more peptides, antibodies, antibody fragments, aptamers, polymers and so on.

In some embodiments, the first strand is at least 1 nt longer than the second strand. In a further embodiment, the first strand is at least 2, 3, 4, 5, 6, 7, 8, 9, 10, 11, 12, 13, 14, 15, 16, 17, 18, 19, or 20 nt longer than the second strand. In another embodiment, the first strand is 20-100 nt longer than the second strand. In a further embodiment, the first strand is 2-12 nt longer than the second strand. In an even further embodiment, the first strand is 3-10 nt longer than the second strand.

In some embodiments, the first strand, or the long strand, has a length of 5-100 nt, or preferably 10-30 or 12-30 nt, or more preferably 15-28 nt. In one embodiment, the first strand is 21 nucleotides in length. In some embodiments, the second strand, or the short strand, has a length of 3-30 nt, or preferably 3-29 nt or 10-26 nt, or more preferably 12-26 nt. In some embodiments, the second strand has a length of 15 nucleotides.

In some embodiments, the double-stranded region has a length of 3-98 basepairs (bp). In a further embodiment, the double-stranded region has a length of 5-28 bp. In an even further embodiment, the double-stranded region has a length of 10-19 bp. The length of the double-stranded region can be 3, 4, 5, 6, 7, 8, 9, 10, 11, 12, 13, 14, 15, 16, 17, 18, 19, 20, 21, 22, 23, 24, 25, 26, 27, 28, 29, or 30 bp.

In some embodiments, the double-stranded region of the RNA molecule does not contain any mismatch or bulge, and the two strands are perfectly complementary to each other in the double-stranded region. In another embodiment, the double-stranded region of the RNA molecule contains mismatch and/or bulge.

In some embodiments, the terminal overhang is 1-10 nucleotides. In a further embodiment, the terminal overhang is 1-8 nucleotides. In another embodiment, the terminal overhang is 3 nt.

The present invention also provides a method of modulating K-Ras gene expression in a cell or an organism (silencing method). The method comprises the steps of contacting said cell or organism with the duplex RNA molecule under conditions wherein selective K-Ras gene silencing can occur, and mediating a selective K-Ras gene silencing effected by the said duplex RNA molecule towards a target K-Ras nucleic acid having a sequence portion substantially corresponding to the double-stranded RNA.

In some embodiments, the contacting step comprises the step of introducing said duplex RNA molecule into a target cell in culture or in an organism in which the selective gene silencing can occur. In a further embodiment, the introducing step comprises transfection, lipofection, infection, electroporation, or other delivery technologies.

In some embodiments, the silencing method is used for determining the function or utility of a gene in a cell or an organism.

The silencing method can be used for modulating the expression of a gene in a cell or an organism. In some embodiments, the gene is associated with a disease, e.g., a human disease or an animal disease, a pathological condition, or an undesirable condition. In some embodiments, the disease is gastric cancer.

The RNA molecules of the present invention can be used for the treatment and or prevention of various diseases or undesirable conditions, including gastric cancer. In some embodiments, the present invention can be used as a cancer therapy or to prevent or to delay the progression of cancer. The RNA molecules of the present invention can be used to silence or knock down k-Ras, which is involved with cell proliferation or other cancer phenotypes.

The present invention provides a method to treat a disease or undesirable condition. The method comprises using the asymmetrical duplex RNA molecule to effect gene silencing of a gene associated with the disease or undesirable condition.

The present invention further provided a pharmaceutical composition. The pharmaceutical comprises (as an active agent) at least one asymmetrical duplex RNA molecule. In some embodiments, the pharmaceutical comprises one or more carriers selected from the group consisting of a pharmaceutical carrier, a positive-charge carrier, a liposome, a protein carrier, a polymer, a nanoparticle, a nanoemulsion, a lipid, and a lipoid. In some embodiments, the composition is for diagnostic applications. In some embodiments, the composition is for therapeutic applications.

The pharmaceutical compositions and formulations of the present invention can be the same or similar to the pharmaceutical compositions and formulations developed for siRNA, miRNA, and antisense RNA (see e.g., de Fougerolles et al., 2007, “Interfering with disease: a progress report on siRNA-based therapeutics.” Nat Rev Drug Discov 6, 443453; Kim and Rossi, 2007, “Strategies for silencing human disease using RNA interference.” Nature reviews 8, 173-184), except for the RNA ingredient. The siRNA, miRNA, and antisense RNA in the pharmaceutical compositions and formulations can be replaced by the duplex RNA molecules of the present disclosure. The pharmaceutical compositions and formulations can also be further modified to accommodate the duplex RNA molecules of the present disclosure.

A “pharmaceutically acceptable salt” or “salt” of the disclosed duplex RNA molecule is a product of the disclosed duplex RNA molecule that contains an ionic bond, and is typically produced by reacting the disclosed duplex RNA molecule with either an acid or a base, suitable for administering to a subject. Pharmaceutically acceptable salt can include, but is not limited to, acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkali earth metal salts such as Mg or Ca, or organic amine salts.

A “pharmaceutical composition” is a formulation containing the disclosed duplex RNA molecules in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed duplex RNA molecule or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of a duplex RNA molecule of this invention include powders, sprays, ointments, pastes, creams, lotions, gels, solutions, patches and inhalants. In one embodiment, the active duplex RNA molecule is mixed under sterile conditions with a pharmaceutically acceptable carrier, and with any preservatives, buffers, or propellants that are required.

The present invention provides a method of treatment comprising administering an effective amount of the pharmaceutical composition to a subject in need. In some embodiments, the pharmaceutical composition is administered via a route selected from the group consisting of iv, sc, topical, po, and ip. In another embodiment, the effective amount is 1 ng to 1 g per day, 100 ng to 1 g per day, or 1 ug to 1 mg per day.

The present invention also provides pharmaceutical formulations comprising a duplex RNA molecule of the present invention in combination with at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, Pa., which is incorporated herein by reference. Examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active duplex RNA molecule, use thereof in the compositions is contemplated. Supplementary active duplex RNA molecules can also be incorporated into the compositions.

A duplex RNA molecule of the present invention is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of a duplex RNA molecule of the present invention (as an active ingredient) with standard pharmaceutical carriers or diluents according to conventional procedures (i.e., by producing a pharmaceutical composition of the invention). These procedures may involve mixing, granulating, compressing, or dissolving the ingredients as appropriate to attain the desired preparation. In another embodiment, a therapeutically effective amount of a duplex RNA molecule of the present invention is administered in a suitable dosage form without standard pharmaceutical carriers or diluents.

Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions containing active duplex RNA molecules of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the active duplex RNA molecules into preparations that can be used pharmaceutically. Of course, the appropriate formulation is dependent upon the route of administration chosen.

A duplex RNA molecule or pharmaceutical composition of the invention can be administered to a subject in many of the well-known methods currently used for chemotherapeutic treatment. For example, for treatment of cancers, a duplex RNA molecule of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not as high as to cause unacceptable side effects. The state of the disease condition (e.g., gastric cancer) and the health of the patient should be closely monitored during and for a reasonable period after treatment.

### EXAMPLES

Examples are provided below to further illustrate different features of the present invention. The examples also illustrate useful methodology for practicing the invention. These examples do not limit the claimed invention.

### Example 1

### In Vitro Potency of aiK-Ras

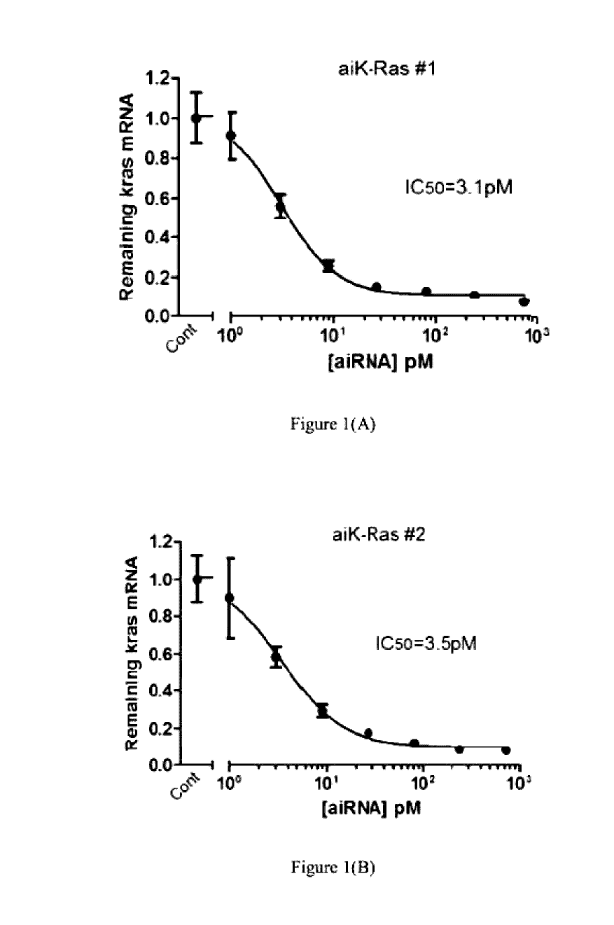

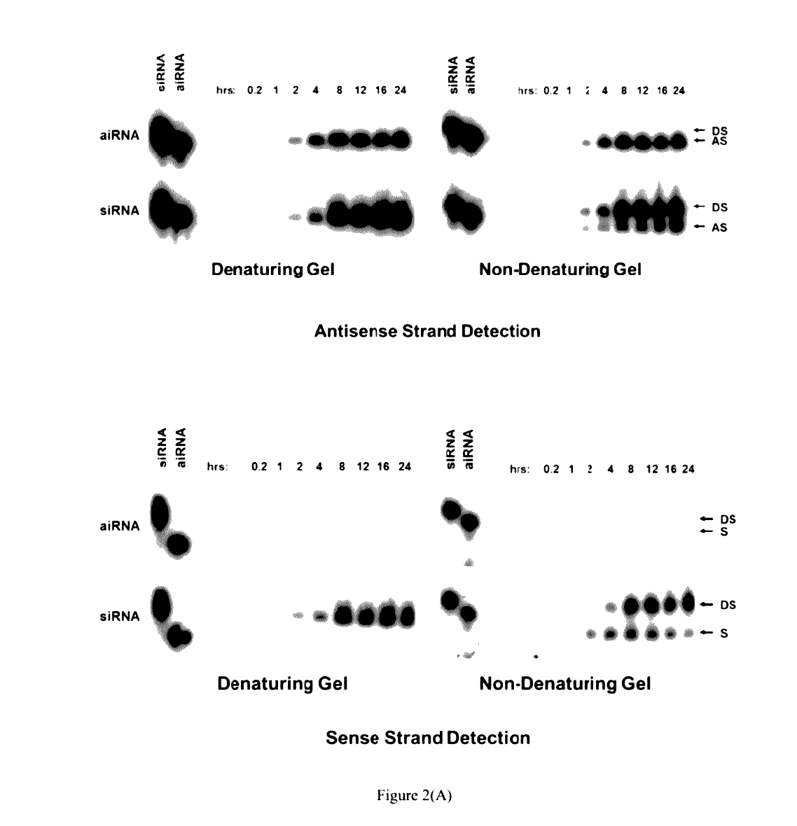

FIG. 1(A) shows an in vitro study in which aiRNA ID NO: 21 (“aiK-Ras #1”) was used to target K-Ras Target SEQ ID NO: 22 to determine the IC₅₀ for aiK-Ras #1. DLD1 cells (ATCC) were transfected with aiK-Ras #1. 48 hours after transfection, cells were collected and RNA was isolated. The IC₅₀ of aiK-Ras #1 was determined by qPCR. Remaining mRNA was standardized to the GAPDH expression level. The IC₅₀ of 3.1 pM indicates that aiK-Ras #1 silences K-Ras gene expression with high potency.

FIG. 1(B) shows an in vitro study in which aiRNA ID NO: 142 (“aiK-Ras #2”) was used to target K-Ras Target SEQ ID NO: 142 to determine the IC₅₀ for aiK-Ras #2. DLD1 cells were transfected with aiK-Ras #2. 48 hours after transfection, cells were collected and RNA was isolated. The IC₅₀ of aiK-Ras #2 was determined by qPCR. Remaining mRNA was standardized to the GAPDH expression level. The IC₅₀ of 3.5 pM indicates that aiK-Ras #1 silences K-Ras gene expression with high potency.

### Example 2

### Reduced Off-Target Effect of aiK-Ras

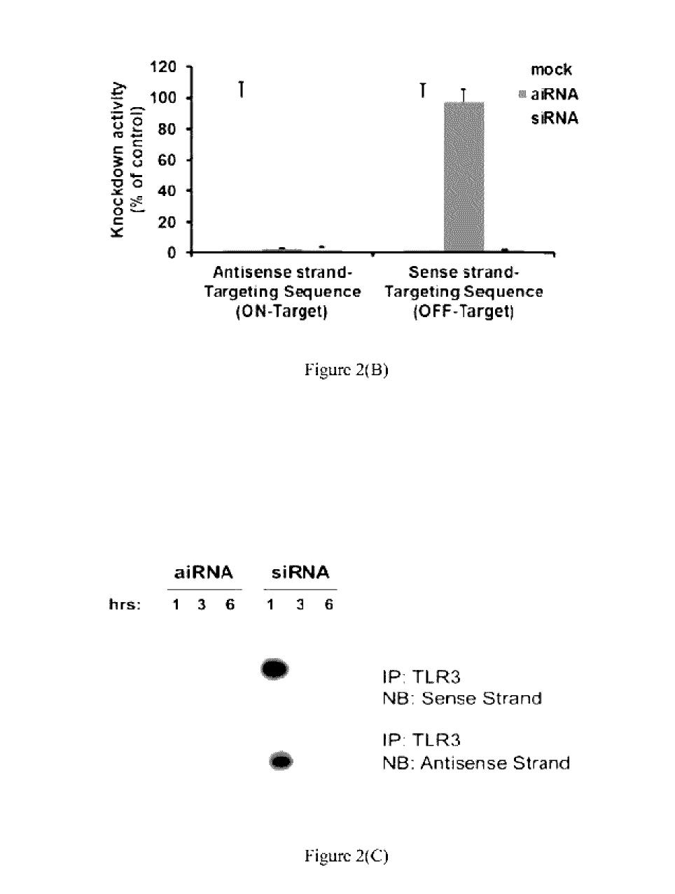

FIG. 2(A) shows detection of siRNA and aiRNA loading to RISC by northern blot analysis. To analyze small RNA RISC loading, HEK293 Flag-Ago2 stable cells were transfected with aiRNA or siRNA duplexes. Cells were lysed at the indicated time points and immunoprecipitated with Flag antibody (Sigma, Catalog # F1804). Immunoprecipitates were washed, RNA isolated from the complex by TRIZOL (Life Technologies, 15596-018) extraction, and loaded on 15% TBE-Urea PAGE or 15% TBE non-denaturing PAGE gels. Following electrophoreses, RNA was transferred to Hybonad-XL Nylon membrane. Then hybridizing the r-P32 labeled detect sense strand or anti-sense strand probe to RNA on the membrane. HEK293 cells (Invivogen, Catalog #293-null) expressing Flag-Ago2 were transfected with siRNA or aiRNA, after which an immunoprecipitation assay was conducted. FLAG-Ago2 HEK 293 cells stably expressing FLAG-Ago2 cells were generated through transient transfection of FLAG-Ago2 neomycin plasmid DNA vectors. After selective neomycin containing medium culture, the monoclonal populations were selected by western blot. Non-denatured gel was used to detect dsRNA.

FIG. 2(B) shows reduced off-target of aiRNA. HeLa cells were transfected with luciferase reporter genes fused with antisense or sense strand-based aiRNA or siRNA target sequences and aiK-Ras#2 or siK-Ras#2 (5 nM). FIG. 2(C) shows that TLR3/RNA complexes were immunoprecipitated with anti-HA antibody (Invivogen, Catalog # ab-hatag). RNA was extracted from the pellet, and northern blot analysis was performed to determine the interaction between aiRNA/siRNA and the TLR3 receptor.

FIGS. 2(A)-(C) show that the asymmetric structure of aiK-Ras #1 and aiK-Ras #2 reduced sense strand mediated off-target effect and LTR3 binding.

### Example 3

### aiK-Ras Sensitivity in K-Ras Mutant Cells

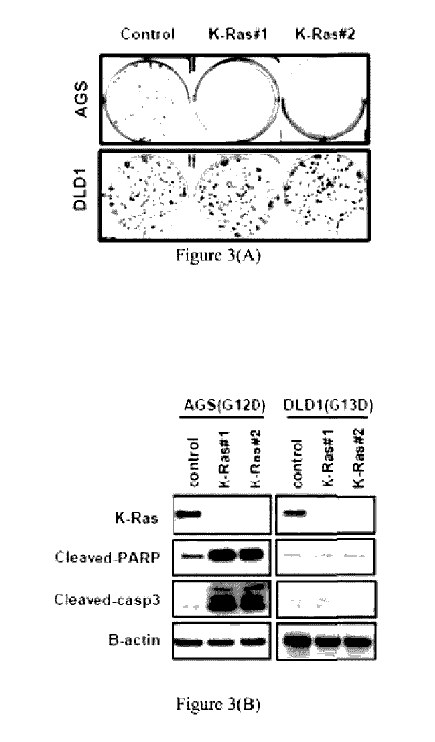

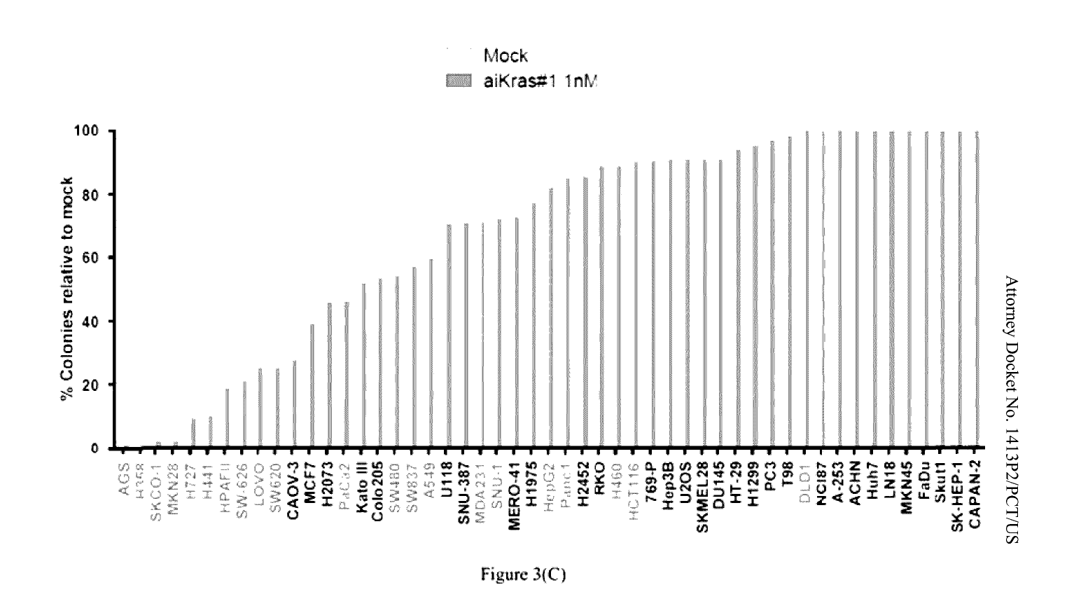

FIG. 3(A) shows colony formation assay in AGS (ATCC) and DLD1 cells transfected with aiK-Ras #1 or aiK-Ras #2. Cells were transfected with 1 nM GFP aiRNA (control; GGTTATGTACAGGAACGCA (SEQ ID NO: 956)) or 1 nM aiK-Ras #1 or aiK-Ras #2 for 24 hours. Cells were then trypsinized and re-plated on 6-well plates at 500-2000 cells/well to determine the colony formation ability of the cells. After 11-14 days, colonies were stained with Giemsa stain and were counted. For the western blot analysis, cells were washed with ice-cold PBS and lysed in lysis buffer [50 mM Hepes (pH 7.5), 1% Nonidet P-40, 150 mM NaCl, 1 mM EDTA, and 1×Halt Protease Inhibitor Cocktail (Thermo Scientefic, Catalog #87786)]. Soluble protein (10 μg) was separated by SDS/PAGE and transferred to PVDF membrane. Primary antibodies against were used in this study. The antigen-antibody complexes were visualized by enhanced chemiluminescence (BioRad, Catalog #170-5060).

FIG. 3(B) shows western blot analysis of lysate from AGS and DLD1, and the transfection effects of aiK-Ras #1 and aiK-Ras #2 on K-Ras expression, cleaved caspase 3, and cleaved PARP.

FIG. 3(C) shows colony formation assay results in a large cell panel. All cell lines in the panel were obtained from ATCC. Cells harboring K-Ras mutant are highlighted.

### Example 4

### Correlation Between aiK-Ras Sensitivity and K-Ras Amplification

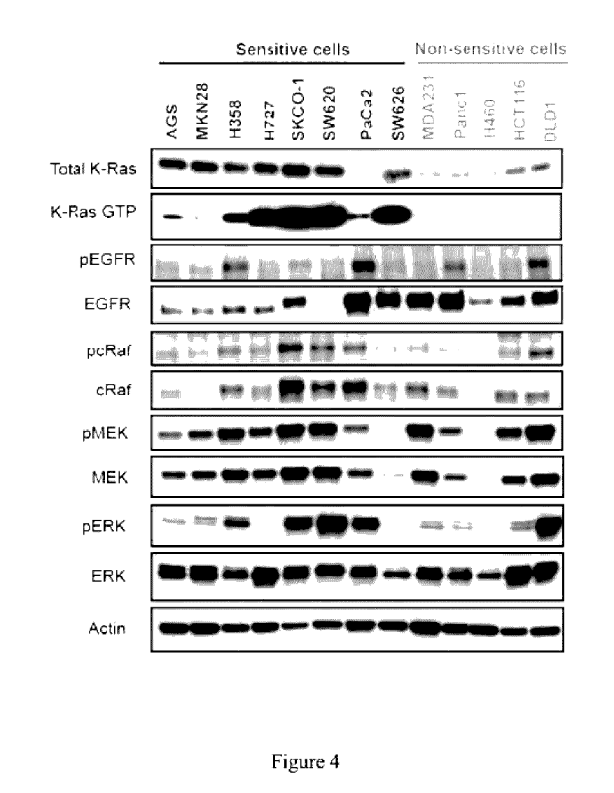

FIG. 4 shows western blot analysis of K-Ras and EGFR-RAS pathway molecules. Lysate (10 μg/lane) was loaded and total and phosphorylated forms of EGFR, cRaf, MEK, and ERK were detected. Activated form of K-Ras (K-Ras GTP) was affinity-purified from cell lysate using GST-Raf-RBD and analyzed by western blotting with K-Ras antibody. The following antibodies were used for western blot: Actin (Sigma, Catalog # A5316) K-RAS (Santa Cruz, sc30 and Cell signaling, Catalog #8955), Cleaved PARP (Cell Signaling, Catalog #5625), Cleaved Caspase-3 (Cell Signaling, Catalog #9664), Phospho-EGF Receptor (Cell Signaling, Catalog #3777), EGF Receptor (Cell Signaling, Catalog #4267), Phospho-c-Raf (Cell signaling, Catalog #9427), c-Raf (Cell Signaling, Catalog #9422), Phospho-MEK1/2 (Cell Signaling, Catalog #9154), MEK1/2 (Cell Signaling, Catalog #8727), Phospho-p44/42 MAPK (Erk1/2) (Cell Signaling, Catalog #4370), p44/42 MAPK (Erk1/2) (Cell Signaling, Catalog #4695), Jagged1 (Cell Signaling, Catalog #2620), Notch1 (Cell Signaling, Catalog #3608), c-Myc (Cell Signaling, Catalog #5605). RBD pulldown was performed using a Ras Activation Kit (Abcam, Catalog # ab128504) according to the manufacturer's protocol. Precipitations were blotted for K-Ras (Santa Cruz, Catalog # sc30). Actin (Sigma, Catalog # A5316) was blotted as loading control. FIG. 4 shows that aiK-Ras sensitivity correlates with K-Ras amplification, and not with the activation state of the Ras pathway molecules.

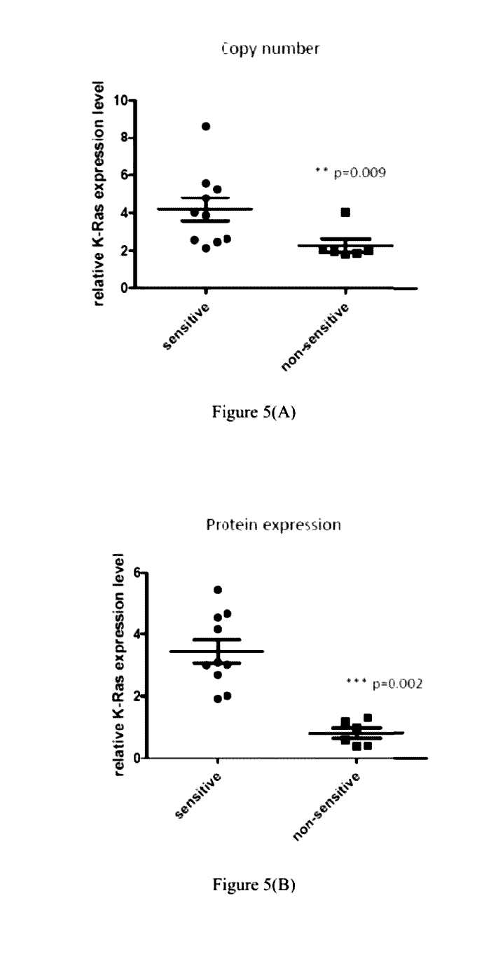

FIG. 5(A) shows that aiK-Ras sensitivity was correlated with K-Ras amplification in K-Ras mutant large cell panel. All cell lines in the panel were obtained from ATCC. Copy number of K-Ras was analyzed by qPCR. Statistical difference was determined by two-sided Mann-Whitney's U test. Difference with p<0.05 was considered statistically significant.

FIG. 5(B) shows that aiK-Ras sensitivity was correlated with K-Ras amplification in K-Ras mutant large cell panel. K-Ras protein expression level was measured by western blot. Band of western blot was quantified by Image Lab (Biorad). Statistical difference was determined by two-sided Mann-Whitney's U test. Difference with p<0.05 was considered statistically significant.

FIGS. 3(A)-(C) and 5(A)-(B) show that aiK-Ras sensitivity varies in K-Ras mutant cells and it correlates with K-Ras copy number.

### Example 6

### Effect of aiK-Ras on CSC-Like Phenotype in Sensitive Cell Lines

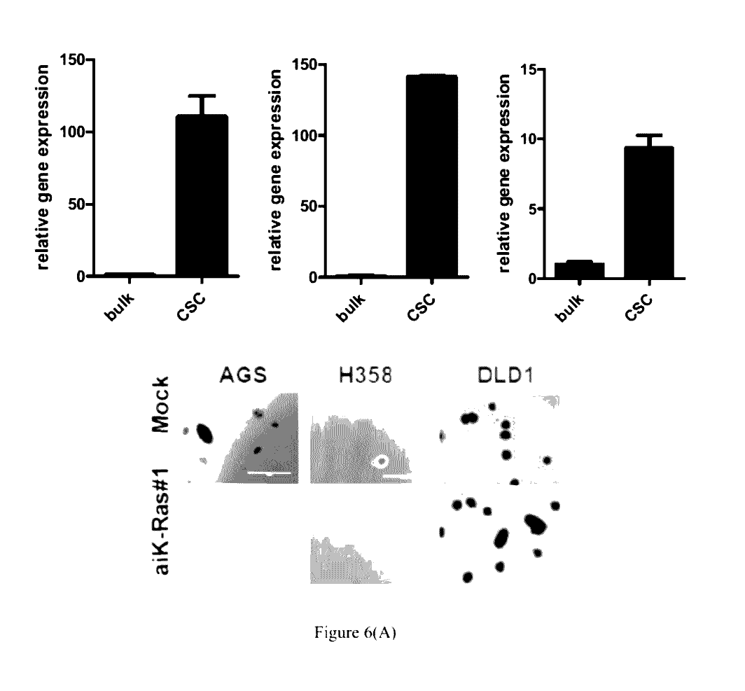

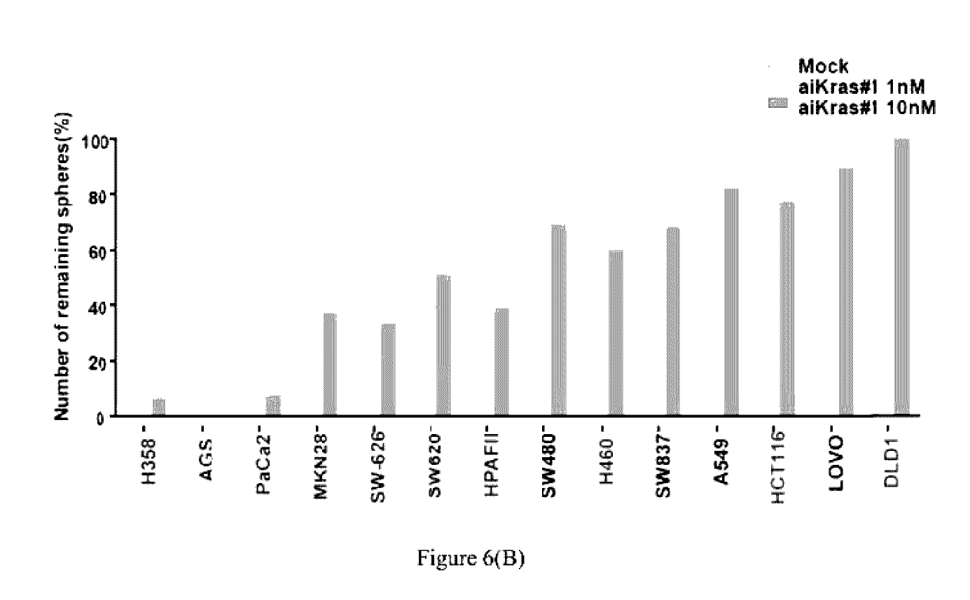

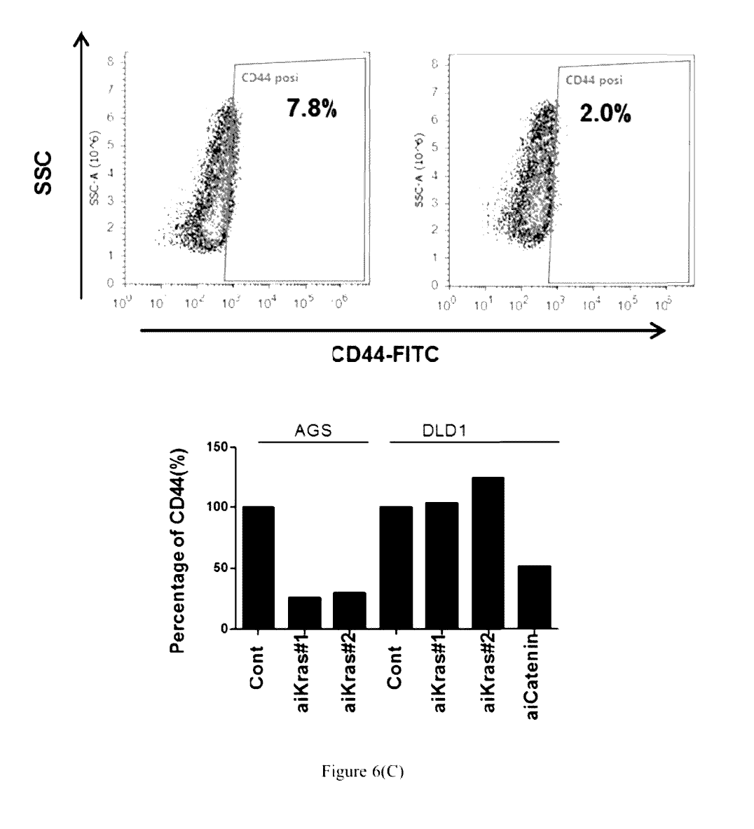

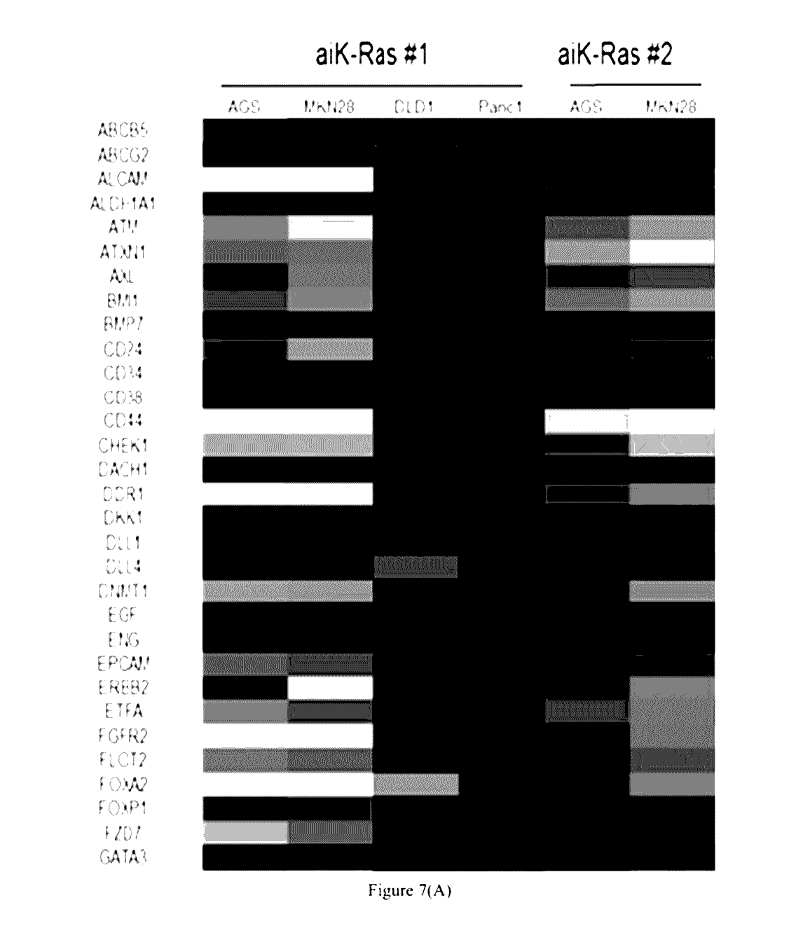

FIG. 6(A) shows stemness gene expression in CSC culture. AGS cells were cultured in CSC medium [DMEM nutrient mixture F-12 (DMEM/F-12, Life technologies, Catalog #11320-033) containing B-27 supplement (Life Technologies, Catalog #17504-044), 20 ng/mL EGF (R&D Systems, Catalog #236-EG), 10 ng/mL FGF (R&D Systems, Catalog #233-FB), and 1% penicillin/streptomycin] for 2 weeks. Nanog, Oct4, and Sox2 gene expression of CSC spheres was quantified by qPCR.

FIG. 6(B) shows the results of sphere formation assay in various cell lines. For the sphere formation assay, agarose coated plates were prepared to dispense autoclaved 0.5% agar and aspirated immediately. Transfected cells were trypsinized and counted, then diluted to 2000 cells/100 uL of 1×CSC medium. 1.9 mL of warmed CSC medium including 0.33% agarose (Sigma type VII, Catalog # A-4018) was added to the cells in CSC medium for final agarose concentration of 0.3%. The plate was placed at 4° C. for 10 minutes to cool. The plate was placed 10 minutes at room temperature and 1 mL of CSC medium was added to the top layer. The plate was incubated in a 37° C./5% CO₂ incubator for 18-25 days. To count spheres, CSC medium was aspirated and Crystal violet (EMD, Catalog #192-12) solution in PBS were added and incubated for 1 hour at room temperature to stain spheres.

Cells were trypsinized and re-plated in CSC medium/3% soft agar onto agar coated 6-well plates at 2000 cells/well to determine the sphere formation ability of the cells. After 18-25 days, spheres were stained with crystal violet, and the number of spheres was counted.

FIG. 6(C) shows depletion of CD44-high population in AGS and DLD1 cells with aiK-Ras #1 and aiK-Ras #2. CD44 expression was detected by flow cytometry, wherein AGS and DLD1 cells were stained with PE conjugated anti-CD44 (BD Pharmingen, Catalog #555479) in Stain Buffer (BD Pharmingen, Catalog #554657) on ice for 45 minutes and washed once with Stain Buffer. CD44 positive population was detected with flow cytometry (Attune Acoustic Focusing Cytometer, Life technologies).

FIGS. 6(A)-(C) show that aiK-Ras according to the present invention modulate CSC-like phenotype in sensitive cell lines.

### Example 7

### Effect of K-Ras Knockdown on CSC-Related Gene Expression Patterns

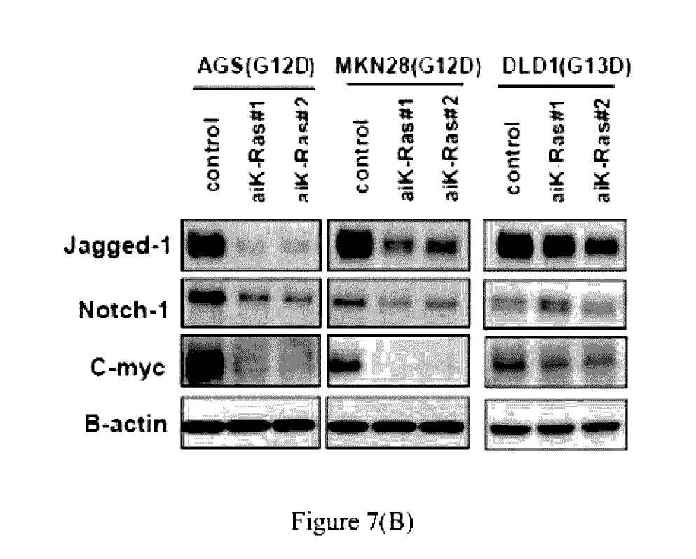

FIG. 7(A) shows heat map of CSC-related genes in cancer cells transfected with aiK-Ras. Cells were transfected with 1 nM control aiRNA or aiK-Ras #1 for 48 hours. Real-time PCR was performed on total RNA using specific validated primers for 84 CSC-related genes with RT2 Profiler PCR array. The fold change in gene expression was calculated as the ratio between aiK-Ras #1 and the control aiRNA samples. FIG. 7(B) shows confirmation of down-regulated Notch signaling by western blot. Table 3 below summarizes the genes down-regulated >3 fold with aiK-Ras #1 corresponding to the heat map as shown in FIG. 7(A)

TABLE 3

| | AGS | MKN28 |
|---|---|---|
| Gene symbol | Fold change | Fold change |
| NOTCH1 | −7.87 | −4.07 |
| SOX2 | −5.49 | −3.97 |
| PTCH1 | −4.94 | −7.04 |
| FOXA2 | −4.85 | −7.35 |
| FGFR2 | −4.29 | −3.67 |
| JAG1 | −4.16 | −3.51 |
| ALCAM | −3.64 | −3.11 |
| MYC | −3.51 | −3.15 |
| ITGA2 | −3.36 | −7.98 |

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A method for treating cancer in a subject in need thereof comprising administering to the subject a duplex RNA molecule comprising
(i) a first strand comprising a nucleotide sequence with a length from 18-23 nucleotides, wherein the nucleotide sequence of the first strand is substantially complementary to a target K-Ras mRNA sequence, and
(ii) a second strand comprising a nucleotide sequence with a length from 12-17 nucleotides, wherein the second strand is substantially complementary to the first strand, and forms a double-stranded region with the first strand,
wherein the first strand has a 3′-overhang from 1-9 nucleotides, and a 5′-overhang from 0-8 nucleotides, and
wherein the duplex RNA molecule is capable of effecting selective K-Ras gene silencing.

2. (canceled)

3. A method for treating cancer in a selected patient population comprising the steps of:
(a) measuring an expression level of mutant K-Ras protein in a biological sample obtained from a patient candidate diagnosed with a cancer and
confirming that the patient candidate's mutant K-Ras protein expression level is above a benchmark level;
or
measuring a level of mutant K-Ras gene amplification in a biological sample obtained from a patient candidate diagnosed with a cancer and confirming that the patient candidates's mutant K-Ras gene amplification level is above a benchmark level; and
(b) administering to the patient candidate a duplex RNA molecule comprising
(i) a first strand comprising a nucleotide sequence with a length from 18-23 nucleotides, wherein the nucleotide sequence of the first strand is substantially complementary to a target K-Ras mRNA sequence, and
(ii) a second strand comprising a nucleotide sequence with a length from 12-17 nucleotides,
wherein the second strand is substantially complementary to the first strand, and forms a double-stranded region with the first strand,
wherein the first strand has a 3′-overhang from 1-9 nucleotides, and a 5′-overhang from 0-8 nucleotides, and
wherein the duplex RNA molecule is capable of effecting selective K-Ras gene silencing.

4. The method of claim 1, wherein the cancer is gastric cancer, or the subject is suffering from or predisposed to gastric cancer.

5. (canceled)

6. (canceled)

7. The method of claim 1, wherein the first strand has a length of 21 nucleotides.

8. (canceled)

9. The method of claim 7, wherein the second strand has a length of 15 nucleotides.

10. (canceled)

11. (canceled)

12. The method of claim 1, wherein the duplex RNA molecule contains at least one modified nucleotide or its analogue.

13. (canceled)

14. (canceled)

15. The method of claim 1, wherein the first strand comprises an antisense strand sequence selected from the group consisting of SEQ ID NOs: 638-955.

16. The method of claim 1, wherein the second strand comprises a sense strand sequence selected from the group consisting of SEQ ID NOs: 320-637.

17. (canceled)

18. (canceled)

19. A duplex RNA molecule comprising
(i) a first strand comprising a nucleotide sequence with a length from 18-23 nucleotides, wherein the nucleotide sequence of the first strand is substantially complementary to a target K-Ras mRNA sequence, and
(ii) a second strand comprising a nucleotide sequence with a length from 12-17 nucleotides,
wherein the second strand is substantially complementary to the first strand, and forms a double-stranded region with the first strand,
wherein the first strand has a 3′-overhang from 1-9 nucleotides, and a 5′-overhang from 0-8 nucleotides, and
wherein said duplex RNA molecule is capable of effecting selective K-Ras gene silencing.

20. The duplex RNA molecule of claim 19, wherein the nucleotide sequence of the first strand comprises a sequence that is at least 70% complementary to the target K-Ras mRNA sequence.

21. The duplex RNA molecule of claim 19, wherein the first strand has a length from 19-23 nucleotides.

22. The duplex RNA molecule of claim 19, wherein the first strand has a length of 21 nucleotides.

23. (canceled)

24. The duplex RNA molecule of claim 22, wherein the second strand has a length of 15 nucleotides.

25. The duplex RNA molecule of claim 24, wherein the first strand has a 3′-overhang of 2-4 nucleotides.

26. (canceled)

27. The duplex RNA molecule of claim 19, wherein the duplex RNA molecule contains at least one modified nucleotide or its analogue.

28. The duplex RNA molecule of claim 27, wherein the at least one modified nucleotide or its analogue is sugar-, backbone-, and/or base-modified ribonucleotide.

29. The duplex RNA molecule of claim 28, wherein the backbone-modified ribonucleotide has a modification in a phosphodiester linkage with another ribonucleotide.

30. The duplex RNA molecule of claim 19, wherein the first strand comprises an antisense strand sequence selected from the group consisting of SEQ ID NOs: 638-955.

31. The duplex RNA molecule of claim 19, wherein the second strand comprises a sense strand sequence selected from the group consisting of SEQ ID NOs: 320-637.

32. (canceled)

33. A method for treating cancer in a subject in need thereof, comprising inhibiting K-Ras gene expression or K-Ras activity in the subject, wherein inhibiting K-Ras gene expression or K-Ras activity inhibits the survival and/or proliferation of cancer stem cells (CSCs) in the subject.

34. (canceled)

35. The method of claim 33, wherein inhibiting K-Ras gene expression or K-Ras activity comprises administering to a subject in need thereof a duplex RNA molecule comprising
(i) a first strand comprising a nucleotide sequence with a length from 18-23 nucleotides, wherein the nucleotide sequence of the first strand is substantially complementary to a target K-Ras mRNA sequence, and
(ii) a second strand comprising a nucleotide sequence with a length from 12-17 nucleotides,
wherein the second strand is substantially complementary to the first strand, and forms a double-stranded region with the first strand,
wherein the first strand has a 3′-overhang from 1-9 nucleotides, and a 5′-overhang from 0-8 nucleotides, and
wherein said duplex RNA molecule is capable of effecting selective K-Ras gene silencing.


SEQUENCE LISTING (956 sequences)

SEQ ID NO: 1 - DNA, 5765 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 2 - DNA, 19 bp, Homo sapiens
ggccagttatagcttatta

SEQ ID NO: 3 - DNA, 19 bp, Homo sapiens
ggtcctagtaggaaataaa

SEQ ID NO: 4 - DNA, 19 bp, Homo sapiens
ggcagacccagtatgaaat

SEQ ID NO: 5 - DNA, 19 bp, Homo sapiens
ggtgtgccaagacattaat

SEQ ID NO: 6 - DNA, 19 bp, Homo sapiens
ggactcttcttccatatta

SEQ ID NO: 7 - DNA, 19 bp, Homo sapiens
ggcaatggaaactattata

SEQ ID NO: 8 - DNA, 19 bp, Homo sapiens
gcagttgattacttcttat

SEQ ID NO: 9 - DNA, 19 bp, Homo sapiens
ggacttagcaagaagttat

SEQ ID NO: 10 - DNA, 19 bp, Homo sapiens
gctcagcacaatctgtaaa

SEQ ID NO: 11 - DNA, 19 bp, Homo sapiens
ctcctttccactgctatta

SEQ ID NO: 12 - DNA, 19 bp, Homo sapiens
gttggtgtgaaacaaatta

SEQ ID NO: 13 - RNA, 19 nt, Homo sapiens
cgauacagcuaauucagaa

SEQ ID NO: 14 - RNA, 19 nt, Homo sapiens
cagcuaauucagaaucauu

SEQ ID NO: 15 - RNA, 19 nt, Homo sapiens
gcuaauucagaaucauuuu

SEQ ID NO: 16 - RNA, 19 nt, Homo sapiens
cgaauaugauccaacaaua

SEQ ID NO: 17 - DNA, 19 bp, Homo sapiens
cctggtaacagtaatacat

SEQ ID NO: 18 - DNA, 19 bp, Homo sapiens
gctcaggacttagcaagaa

SEQ ID NO: 19 - DNA, 19 bp, Homo sapiens
ctctgtgccagctctataa

SEQ ID NO: 20 - DNA, 19 bp, Homo sapiens
gggctatatttacatgcta

SEQ ID NO: 21 - DNA, 19 bp, Homo sapiens
cctgtctcttggatattct

SEQ ID NO: 22 - DNA, 19 bp, Homo sapiens
ggagggctttctttgtgta

SEQ ID NO: 23 - DNA, 19 bp, Homo sapiens
gtggatatctccatgaagt

SEQ ID NO: 24 - DNA, 19 bp, Homo sapiens
caccattatagagaacaaa

SEQ ID NO: 25 - DNA, 19 bp, Homo sapiens
ggtctgtaatatcttacta

SEQ ID NO: 26 - DNA, 19 bp, Homo sapiens
ccttgacgatacagctaat

SEQ ID NO: 27 - DNA, 19 bp, Homo sapiens
gctgatgctttgaacatct

SEQ ID NO: 28 - DNA, 19 bp, Homo sapiens
catccctgatgaatgtaaa

SEQ ID NO: 29 - RNA, 19 nt, Homo sapiens
guguauuugccauaaauaa

SEQ ID NO: 30 - RNA, 19 nt, Homo sapiens
cauaaauaauacuaaauca

SEQ ID NO: 31 - RNA, 19 nt, Homo sapiens
cuaaaucauuugaagauau

SEQ ID NO: 32 - RNA, 19 nt, Homo sapiens
gaagauauucaccauuaua

SEQ ID NO: 33 - DNA, 19 bp, Homo sapiens
tggtttaacaaaagatttg

SEQ ID NO: 34 - DNA, 19 bp, Homo sapiens
tgtcctaatatatacatag

SEQ ID NO: 35 - DNA, 19 bp, Homo sapiens
tgaaaaagaaactgaatac

SEQ ID NO: 36 - DNA, 19 bp, Homo sapiens
ctcagcacaatctgtaaat

SEQ ID NO: 37 - DNA, 19 bp, Homo sapiens
gctctttcatagtataact

SEQ ID NO: 38 - DNA, 19 bp, Homo sapiens
gtgctagtgtggtctgtaa

SEQ ID NO: 39 - DNA, 19 bp, Homo sapiens
ctagtgtggtctgtaatat

SEQ ID NO: 40 - DNA, 19 bp, Homo sapiens
gcagacgtatattgtatca

SEQ ID NO: 41 - DNA, 19 bp, Homo sapiens
gttcccaagtaggcattct

SEQ ID NO: 42 - DNA, 19 bp, Homo sapiens
ggacgaatatgatccaaca

SEQ ID NO: 43 - DNA, 19 bp, Homo sapiens
gaagcaagtagtaattgat

SEQ ID NO: 44 - DNA, 19 bp, Homo sapiens
gcttcctgatgatgattct

SEQ ID NO: 45 - DNA, 19 bp, Homo sapiens
cctgacctcaagtgattca

SEQ ID NO: 46 - DNA, 19 bp, Homo sapiens
gcctccctaccttccacat

SEQ ID NO: 47 - DNA, 19 bp, Homo sapiens
gccatttccttttcacatt

SEQ ID NO: 48 - DNA, 19 bp, Homo sapiens
gacgtatattgtatcattt

SEQ ID NO: 49 - DNA, 19 bp, Homo sapiens
ggccatttccttttcacat

SEQ ID NO: 50 - DNA, 19 bp, Homo sapiens
gggggagaattctagaaat

SEQ ID NO: 51 - DNA, 19 bp, Homo sapiens
gttcccaagtaggcattct

SEQ ID NO: 52 - DNA, 19 bp, Homo sapiens
ggacgaatatgatccaaca

SEQ ID NO: 53 - DNA, 19 bp, Homo sapiens
gaagcaagtagtaattgat

SEQ ID NO: 54 - DNA, 19 bp, Homo sapiens
gcttcctgatgatgattct

SEQ ID NO: 55 - DNA, 19 bp, Homo sapiens
gaactgtactactcctaat

SEQ ID NO: 56 - DNA, 19 bp, Homo sapiens
cctgacctcaagtgattca

SEQ ID NO: 57 - DNA, 19 bp, Homo sapiens
gtccttaggtagtgctagt

SEQ ID NO: 58 - DNA, 19 bp, Homo sapiens
gtgtcagactgctctttca

SEQ ID NO: 59 - DNA, 19 bp, Homo sapiens
gactgctctttcatagtat

SEQ ID NO: 60 - DNA, 19 bp, Homo sapiens
cctgatgaatgtaaagtta

SEQ ID NO: 61 - DNA, 19 bp, Homo sapiens
caagtctgatccatattta

SEQ ID NO: 62 - DNA, 19 bp, Homo sapiens
gatgagcaaagatggtaaa

SEQ ID NO: 63 - DNA, 19 bp, Homo sapiens
caagaggtgaagtttatat

SEQ ID NO: 64 - DNA, 19 bp, Homo sapiens
ggtagggtgttaagactta

SEQ ID NO: 65 - DNA, 19 bp, Homo sapiens
ctaggcatcatgtcctata

SEQ ID NO: 66 - DNA, 19 bp, Homo sapiens
gagtgaatgttcccaagta

SEQ ID NO: 67 - DNA, 19 bp, Homo sapiens
cctagtaggaaataaatgt

SEQ ID NO: 68 - DNA, 19 bp, Homo sapiens
gcctgaactagttcacaga

SEQ ID NO: 69 - DNA, 19 bp, Homo sapiens
ccagaaatcttcatgcaat

SEQ ID NO: 70 - DNA, 19 bp, Homo sapiens
gctgtggatatctccatga

SEQ ID NO: 71 - DNA, 19 bp, Homo sapiens
ggaagcaagtagtaattga

SEQ ID NO: 72 - DNA, 19 bp, Homo sapiens
cagacgtatattgtatcat

SEQ ID NO: 73 - DNA, 19 bp, Homo sapiens
gccttgacgatacagctaa

SEQ ID NO: 74 - DNA, 19 bp, Homo sapiens
gaaggtgacttaggttcta

SEQ ID NO: 75 - DNA, 19 bp, Homo sapiens
ggctagttctcttaacact

SEQ ID NO: 76 - DNA, 19 bp, Homo sapiens
gtgtatgtcagatattcat

SEQ ID NO: 77 - DNA, 19 bp, Homo sapiens
gaacctttgagctttcata

SEQ ID NO: 78 - DNA, 19 bp, Homo sapiens
cagacaaggaaacttctat

SEQ ID NO: 79 - DNA, 19 bp, Homo sapiens
cttcgatcaagctacttta

SEQ ID NO: 80 - DNA, 19 bp, Homo sapiens
gagtgccaatttcttacta

SEQ ID NO: 81 - DNA, 19 bp, Homo sapiens
gctgacaaatcaagagcat

SEQ ID NO: 82 - DNA, 19 bp, Homo sapiens
gtcatctcaaactcttagt

SEQ ID NO: 83 - DNA, 19 bp, Homo sapiens
gatgatgccttctatacat

SEQ ID NO: 84 - DNA, 19 bp, Homo sapiens
ctggtatgaatagacagaa

SEQ ID NO: 85 - DNA, 19 bp, Homo sapiens
cactgagtcacatcagaaa

SEQ ID NO: 86 - DNA, 19 bp, Homo sapiens
gttgtcaccattgcacaat

SEQ ID NO: 87 - DNA, 19 bp, Homo sapiens
gtcaagctcagcacaatct

SEQ ID NO: 88 - DNA, 19 bp, Homo sapiens
gggattattatagcaacca

SEQ ID NO: 89 - DNA, 19 bp, Homo sapiens
ctaggaagaaggtgactta

SEQ ID NO: 90 - DNA, 19 bp, Homo sapiens
ggactctgaagatgtacct

SEQ ID NO: 91 - DNA, 19 bp, Homo sapiens
ctgagtagctgggattaca

SEQ ID NO: 92 - DNA, 19 bp, Homo sapiens
catgagttcttgaagaata

SEQ ID NO: 93 - DNA, 19 bp, Homo sapiens
gtggacgaatatgatccaa

SEQ ID NO: 94 - DNA, 19 bp, Homo sapiens
ctgtggatatctccatgaa

SEQ ID NO: 95 - DNA, 19 bp, Homo sapiens
gacaaggaaacttctatgt

SEQ ID NO: 96 - DNA, 19 bp, Homo sapiens
gaatatagcagacgtatat

SEQ ID NO: 97 - DNA, 19 bp, Homo sapiens
cgatcaagctactttatgt

SEQ ID NO: 98 - DNA, 19 bp, Homo sapiens
cctggtatgaatagacaga

SEQ ID NO: 99 - DNA, 19 bp, Homo sapiens
cagtaatacattccattgt

SEQ ID NO: 100 - DNA, 19 bp, Homo sapiens
gtgaacctttgagctttca

SEQ ID NO: 101 - DNA, 19 bp, Homo sapiens
gctgaaaatgactgaatat

SEQ ID NO: 102 - DNA, 19 bp, Homo sapiens
ctgaaaatgactgaatata

SEQ ID NO: 103 - DNA, 19 bp, Homo sapiens
gaaaatgactgaatataaa

SEQ ID NO: 104 - DNA, 19 bp, Homo sapiens
cgatacagctaattcagaa

SEQ ID NO: 105 - DNA, 19 bp, Homo sapiens
cagctaattcagaatcatt

SEQ ID NO: 106 - DNA, 19 bp, Homo sapiens
gctaattcagaatcatttt

SEQ ID NO: 107 - DNA, 19 bp, Homo sapiens
gaatcattttgtggacgaa

SEQ ID NO: 108 - DNA, 19 bp, Homo sapiens
cgaatatgatccaacaata

SEQ ID NO: 109 - DNA, 19 bp, Homo sapiens
gatccaacaatagaggatt

SEQ ID NO: 110 - DNA, 19 bp, Homo sapiens
caacaatagaggattccta

SEQ ID NO: 111 - DNA, 19 bp, Homo sapiens
ggattcctacaggaagcaa

SEQ ID NO: 112 - DNA, 19 bp, Homo sapiens
cctacaggaagcaagtagt

SEQ ID NO: 113 - DNA, 19 bp, Homo sapiens
ctacaggaagcaagtagta

SEQ ID NO: 114 - DNA, 19 bp, Homo sapiens
caggaagcaagtagtaatt

SEQ ID NO: 115 - DNA, 19 bp, Homo sapiens
gcaagtagtaattgatgga

SEQ ID NO: 116 - DNA, 19 bp, Homo sapiens
gtagtaattgatggagaaa

SEQ ID NO: 117 - DNA, 19 bp, Homo sapiens
gatggagaaacctgtctct

SEQ ID NO: 118 - DNA, 19 bp, Homo sapiens
gagaaacctgtctcttgga

SEQ ID NO: 119 - DNA, 19 bp, Homo sapiens
gaaacctgtctcttggata

SEQ ID NO: 120 - DNA, 19 bp, Homo sapiens
gtctcttggatattctcga

SEQ ID NO: 121 - DNA, 19 bp, Homo sapiens
ctcttggatattctcgaca

SEQ ID NO: 122 - DNA, 19 bp, Homo sapiens
cttggatattctcgacaca

SEQ ID NO: 123 - DNA, 19 bp, Homo sapiens
ggatattctcgacacagca

SEQ ID NO: 124 - DNA, 19 bp, Homo sapiens
ctcgacacagcaggtcaag

SEQ ID NO: 125 - DNA, 19 bp, Homo sapiens
gcaggtcaagaggagtaca

SEQ ID NO: 126 - DNA, 19 bp, Homo sapiens
caagaggagtacagtgcaa

SEQ ID NO: 127 - DNA, 19 bp, Homo sapiens
gaggagtacagtgcaatga

SEQ ID NO: 128 - DNA, 18 bp, Homo sapiens
caatgagggaccagtaca

SEQ ID NO: 129 - DNA, 19 bp, Homo sapiens
ggaccagtacatgaggact

SEQ ID NO: 130 - DNA, 19 bp, Homo sapiens
gggagggctttctttgtgt

SEQ ID NO: 131 - DNA, 19 bp, Homo sapiens
gagggctttctttgtgtat

SEQ ID NO: 132 - DNA, 19 bp, Homo sapiens
gggctttctttgtgtattt

SEQ ID NO: 133 - DNA, 19 bp, Homo sapiens
ctttgtgtatttgccataa

SEQ ID NO: 134 - DNA, 18 bp, Homo sapiens
gtatttgccataaataat

SEQ ID NO: 135 - DNA, 19 bp, Homo sapiens
ctaaatcatttgaagatat

SEQ ID NO: 136 - DNA, 19 bp, Homo sapiens
gaagatattcaccattata

SEQ ID NO: 137 - DNA, 19 bp, Homo sapiens
ccattatagagaacaaatt

SEQ ID NO: 138 - DNA, 19 bp, Homo sapiens
cattatagagaacaaatta

SEQ ID NO: 139 - DNA, 19 bp, Homo sapiens
gagaacaaattaaaagagt

SEQ ID NO: 140 - DNA, 19 bp, Homo sapiens
gaacaaattaaaagagtta

SEQ ID NO: 141 - DNA, 19 bp, Homo sapiens
gagttaaggactctgaaga

SEQ ID NO: 142 - DNA, 19 bp, Homo sapiens
gttaaggactctgaagatg

SEQ ID NO: 143 - DNA, 19 bp, Homo sapiens
ggactctgaagatgtacct

SEQ ID NO: 144 - DNA, 19 bp, Homo sapiens
gactctgaagatgtaccta

SEQ ID NO: 145 - DNA, 19 bp, Homo sapiens
ctgaagatgtacctatggt

SEQ ID NO: 146 - DNA, 19 bp, Homo sapiens
cctatggtcctagtaggaa

SEQ ID NO: 147 - DNA, 19 bp, Homo sapiens
ctatggtcctagtaggaaa

SEQ ID NO: 148 - DNA, 19 bp, Homo sapiens
gtcctagtaggaaataaat

SEQ ID NO: 149 - DNA, 19 bp, Homo sapiens
gtaggaaataaatgtgatt

SEQ ID NO: 150 - DNA, 19 bp, Homo sapiens
ccttctagaacagtagaca

SEQ ID NO: 151 - DNA, 19 bp, Homo sapiens
ctagaacagtagacacaaa

SEQ ID NO: 152 - DNA, 19 bp, Homo sapiens
gaacagtagacacaaaaca

SEQ ID NO: 153 - DNA, 19 bp, Homo sapiens
caaaacaggctcaggactt

SEQ ID NO: 154 - DNA, 19 bp, Homo sapiens
caggctcaggacttagcaa

SEQ ID NO: 155 - DNA, 19 bp, Homo sapiens
ggctcaggacttagcaaga

SEQ ID NO: 156 - DNA, 19 bp, Homo sapiens
caggacttagcaagaagtt

SEQ ID NO: 157 - DNA, 19 bp, Homo sapiens
cttagcaagaagttatgga

SEQ ID NO: 158 - DNA, 19 bp, Homo sapiens
gcaagaagttatggaattc

SEQ ID NO: 159 - DNA, 19 bp, Homo sapiens
gaagttatggaattccttt

SEQ ID NO: 160 - DNA, 19 bp, Homo sapiens
gttatggaattccttttat

SEQ ID NO: 161 - DNA, 19 bp, Homo sapiens
ggaattccttttattgaaa

SEQ ID NO: 162 - DNA, 19 bp, Homo sapiens
gaaacatcagcaaagacaa

SEQ ID NO: 163 - DNA, 19 bp, Homo sapiens
catcagcaaagacaagaca

SEQ ID NO: 164 - DNA, 19 bp, Homo sapiens
gcaaagacaagacagggtg

SEQ ID NO: 165 - DNA, 19 bp, Homo sapiens
caaagacaagacagggtgt

SEQ ID NO: 166 - DNA, 19 bp, Homo sapiens
gacaagacagggtgttgat

SEQ ID NO: 167 - DNA, 19 bp, Homo sapiens
caagacagggtgttgatga

SEQ ID NO: 168 - DNA, 19 bp, Homo sapiens
cagggtgttgatgatgcct

SEQ ID NO: 169 - DNA, 19 bp, Homo sapiens
ggtgttgatgatgccttct

SEQ ID NO: 170 - DNA, 19 bp, Homo sapiens
gtgttgatgatgccttcta

SEQ ID NO: 171 - DNA, 19 bp, Homo sapiens
gttgatgatgccttctata

SEQ ID NO: 172 - DNA, 19 bp, Homo sapiens
atgatgccttctatacatt

SEQ ID NO: 173 - DNA, 19 bp, Homo sapiens
gatgccttctatacattag

SEQ ID NO: 174 - DNA, 19 bp, Homo sapiens
gccttctatacattagttc

SEQ ID NO: 175 - DNA, 19 bp, Homo sapiens
cttctatacattagttcga

SEQ ID NO: 176 - DNA, 19 bp, Homo sapiens
ctatacattagttcgagaa

SEQ ID NO: 177 - DNA, 19 bp, Homo sapiens
cgagaaattcgaaaacata

SEQ ID NO: 178 - DNA, 19 bp, Homo sapiens
gagaaattcgaaaacataa

SEQ ID NO: 179 - DNA, 19 bp, Homo sapiens
cgaaaacataaagaaaaga

SEQ ID NO: 180 - DNA, 19 bp, Homo sapiens
gaaaacataaagaaaagat

SEQ ID NO: 181 - DNA, 19 bp, Homo sapiens
cataaagaaaagatgagca

SEQ ID NO: 182 - DNA, 19 bp, Homo sapiens
gcaaagatggtaaaaagaa

SEQ ID NO: 183 - DNA, 19 bp, Homo sapiens
caaagatggtaaaaagaag

SEQ ID NO: 184 - DNA, 19 bp, Homo sapiens
gatggtaaaaagaagaaaa

SEQ ID NO: 185 - DNA, 19 bp, Homo sapiens
ggtaaaaagaagaaaaaga

SEQ ID NO: 186 - DNA, 19 bp, Homo sapiens
gtaaaaagaagaaaaagaa

SEQ ID NO: 187 - DNA, 19 bp, Homo sapiens
gaagaaaaagaagtcaaag

SEQ ID NO: 188 - DNA, 19 bp, Homo sapiens
gaaaaagaagtcaaagaca

SEQ ID NO: 189 - DNA, 19 bp, Homo sapiens
gaagtcaaagacaaagtgt

SEQ ID NO: 190 - DNA, 19 bp, Homo sapiens
gtcaaagacaaagtgtgta

SEQ ID NO: 191 - DNA, 19 bp, Homo sapiens
caaagacaaagtgtgtaat

SEQ ID NO: 192 - DNA, 19 bp, Homo sapiens
gacaaagtgtgtaattatg

SEQ ID NO: 193 - DNA, 19 bp, Homo sapiens
caaagtgtgtaattatgta

SEQ ID NO: 194 - DNA, 19 bp, Homo sapiens
caatttgtacttttttctt

SEQ ID NO: 195 - DNA, 19 bp, Homo sapiens
gtacttttttcttaaggca

SEQ ID NO: 196 - DNA, 19 bp, Homo sapiens
cttttttcttaaggcatac

SEQ ID NO: 197 - DNA, 19 bp, Homo sapiens
cttaaggcatactagtaca

SEQ ID NO: 198 - DNA, 19 bp, Homo sapiens
catactagtacaagtggta

SEQ ID NO: 199 - DNA, 19 bp, Homo sapiens
ctagtacaagtggtaattt

SEQ ID NO: 200 - DNA, 19 bp, Homo sapiens
gtacaagtggtaatttttg

SEQ ID NO: 201 - DNA, 19 bp, Homo sapiens
gtggtaatttttgtacatt

SEQ ID NO: 202 - DNA, 19 bp, Homo sapiens
gtaatttttgtacattaca

SEQ ID NO: 203 - RNA, 19 nt, Homo sapiens
guacauuacacuaaauuau

SEQ ID NO: 204 - DNA, 19 bp, Homo sapiens
cattacactaaattattag

SEQ ID NO: 205 - DNA, 19 bp, Homo sapiens
ctaaattattagcatttgt

SEQ ID NO: 206 - DNA, 19 bp, Homo sapiens
gcatttgttttagcattac

SEQ ID NO: 207 - DNA, 19 bp, Homo sapiens
gttttagcattacctaatt

SEQ ID NO: 208 - DNA, 19 bp, Homo sapiens
cctgctccatgcagactgt

SEQ ID NO: 209 - DNA, 19 bp, Homo sapiens
ctgctccatgcagactgtt

SEQ ID NO: 210 - DNA, 19 bp, Homo sapiens
gctccatgcagactgttag

SEQ ID NO: 211 - DNA, 19 bp, Homo sapiens
ccatgcagactgttagctt

SEQ ID NO: 212 - DNA, 20 bp, Homo sapiens
gactgttagcttttacctta

SEQ ID NO: 213 - RNA, 19 nt, Homo sapiens
guuagcuuuuaccuuaaau

SEQ ID NO: 214 - RNA, 19 nt, Homo sapiens
gcuuuuaccuuaaaugcuu

SEQ ID NO: 215 - RNA, 19 nt, Homo sapiens
cuuuuaccuuaaaugcuua

SEQ ID NO: 216 - RNA, 19 nt, Homo sapiens
guuuuuuuuuccucuaagu

SEQ ID NO: 217 - RNA, 19 nt, Homo sapiens
ccaguauucccagaguuuu

SEQ ID NO: 218 - RNA, 19 nt, Homo sapiens
cagaguuuugguuuuugaa

SEQ ID NO: 219 - RNA, 19 nt, Homo sapiens
gaguuuugguuuuugaacu

SEQ ID NO: 220 - RNA, 19 nt, Homo sapiens
cuagcaaugccugugaaaa

SEQ ID NO: 221 - RNA, 19 nt, Homo sapiens
cugugaaaaagaaacugaa

SEQ ID NO: 222 - RNA, 19 nt, Homo sapiens
gugaaaaagaaacugaaua

SEQ ID NO: 223 - RNA, 19 nt, Homo sapiens
cugaauaccuaagauuucu

SEQ ID NO: 224 - RNA, 19 nt, Homo sapiens
gaauaccuaagauuucugu

SEQ ID NO: 225 - RNA, 19 nt, Homo sapiens
caguugauuacuucuuauu

SEQ ID NO: 226 - RNA, 19 nt, Homo sapiens
guugauuacuucuuauuuu

SEQ ID NO: 227 - RNA, 19 nt, Homo sapiens
gauuacuucuuauuuuucu

SEQ ID NO: 228 - RNA, 19 nt, Homo sapiens
cuuauuuuucuuaccaauu

SEQ ID NO: 229 - RNA, 19 nt, Homo sapiens
cuuaccaauugugaauguu

SEQ ID NO: 230 - RNA, 19 nt, Homo sapiens
gaauguuggugugaaacaa

SEQ ID NO: 231 - RNA, 19 nt, Homo sapiens
gugugaaacaaauuaauga

SEQ ID NO: 232 - RNA, 19 nt, Homo sapiens
ccuauucuguguuuuaucu

SEQ ID NO: 233 - RNA, 19 nt, Homo sapiens
cuauucuguguuuuaucua

SEQ ID NO: 234 - RNA, 19 nt, Homo sapiens
cauaaauggauuaauuacu

SEQ ID NO: 235 - RNA, 19 nt, Homo sapiens
cuucuaauugguuuuuacu

SEQ ID NO: 236 - RNA, 19 nt, Homo sapiens
cuaauugguuuuuacugaa

SEQ ID NO: 237 - RNA, 19 nt, Homo sapiens
guuuuuacugaaacauuga

SEQ ID NO: 238 - RNA, 19 nt, Homo sapiens
gcaucauguccuauaguuu

SEQ ID NO: 239 - RNA, 19 nt, Homo sapiens
guucacaaagguuuugucu

SEQ ID NO: 240 - RNA, 19 nt, Homo sapiens
gccauuuccuuuucacauu

SEQ ID NO: 241 - RNA, 19 nt, Homo sapiens
ccauuuccuuuucacauua

SEQ ID NO: 242 - DNA, 19 bp, Homo sapiens
ctccatgcagactgttagc

SEQ ID NO: 243 - DNA, 19 bp, Homo sapiens
catgcagactgttagcttt

SEQ ID NO: 244 - DNA, 19 bp, Homo sapiens
gcagactgttagcttttac

SEQ ID NO: 245 - DNA, 19 bp, Homo sapiens
ctgttagcttttaccttaa

SEQ ID NO: 246 - DNA, 19 bp, Homo sapiens
ccttaaatgcttattttaa

SEQ ID NO: 247 - DNA, 19 bp, Homo sapiens
gacagtggaagtttttttt

SEQ ID NO: 248 - DNA, 19 bp, Homo sapiens
acagtggaagttttttttt

SEQ ID NO: 249 - DNA, 19 bp, Homo sapiens
cctctaagtgccagtattc

SEQ ID NO: 250 - DNA, 19 bp, Homo sapiens
ctaagtgccagtattccca

SEQ ID NO: 251 - DNA, 19 bp, Homo sapiens
gtgccagtattcccagagt

SEQ ID NO: 252 - DNA, 19 bp, Homo sapiens
gccagtattcccagagttt

SEQ ID NO: 253 - DNA, 19 bp, Homo sapiens
ccagtattcccagagtttt

SEQ ID NO: 254 - DNA, 19 bp, Homo sapiens
cagtattcccagagttttg

SEQ ID NO: 255 - DNA, 19 bp, Homo sapiens
gtattcccagagttttggt

SEQ ID NO: 256 - DNA, 19 bp, Homo sapiens
cccagagttttggtttttg

SEQ ID NO: 257 - DNA, 19 bp, Homo sapiens
ccagagttttggtttttga

SEQ ID NO: 258 - DNA, 19 bp, Homo sapiens
agagttttggtttttgaac

SEQ ID NO: 259 - DNA, 19 bp, Homo sapiens
gttttggtttttgaactag

SEQ ID NO: 260 - DNA, 19 bp, Homo sapiens
ggtttttgaactagcaatg

SEQ ID NO: 261 - DNA, 19 bp, Homo sapiens
gaactagcaatgcctgtga

SEQ ID NO: 262 - DNA, 19 bp, Homo sapiens
gcaatgcctgtgaaaaaga

SEQ ID NO: 263 - DNA, 19 bp, Homo sapiens
caatgcctgtgaaaaagaa

SEQ ID NO: 264 - DNA, 19 bp, Homo sapiens
gcctgtgaaaaagaaactg

SEQ ID NO: 265 - DNA, 19 bp, Homo sapiens
cctgtgaaaaagaaactga

SEQ ID NO: 266 - DNA, 19 bp, Homo sapiens
tgaaaaagaaactgaatac

SEQ ID NO: 267 - DNA, 19 bp, Homo sapiens
gaaactgaatacctaagat

SEQ ID NO: 268 - DNA, 19 bp, Homo sapiens
ctgtcttggggtttttggt

SEQ ID NO: 269 - DNA, 19 bp, Homo sapiens
gtcttggggtttttggtgc

SEQ ID NO: 270 - DNA, 19 bp, Homo sapiens
cttggggtttttggtgcat

SEQ ID NO: 271 - DNA, 19 bp, Homo sapiens
gcatgcagtgtttttggtg

SEQ ID NO: 272 - DNA, 19 bp, Homo sapiens
gtttttggtgcatgcagtt

SEQ ID NO: 273 - DNA, 19 bp, Homo sapiens
ccttttcacattagataaa

SEQ ID NO: 274 - DNA, 19 bp, Homo sapiens
cttttcacattagataaat

SEQ ID NO: 275 - DNA, 19 bp, Homo sapiens
gtatgaaatggggattatt

SEQ ID NO: 276 - DNA, 19 bp, Homo sapiens
ccattttggggctatattt

SEQ ID NO: 277 - DNA, 19 bp, Homo sapiens
cattttggggctatattta

SEQ ID NO: 278 - DNA, 19 bp, Homo sapiens
gaaaagattttaacaagta

SEQ ID NO: 279 - DNA, 19 bp, Homo sapiens
caagtataaaaaattctca

SEQ ID NO: 280 - DNA, 19 bp, Homo sapiens
cataggaattaaatgtagt

SEQ ID NO: 281 - DNA, 19 bp, Homo sapiens
gctctttcatagtataact

SEQ ID NO: 282 - DNA, 19 bp, Homo sapiens
ctctttcatagtataactt

SEQ ID NO: 283 - DNA, 19 bp, Homo sapiens
ctttcatagtataacttta

SEQ ID NO: 284 - DNA, 19 bp, Homo sapiens
ctttaaatcttttcttcaa

SEQ ID NO: 285 - DNA, 19 bp, Homo sapiens
cttcaacttgagtctttga

SEQ ID NO: 286 - DNA, 19 bp, Homo sapiens
caacttgagtctttgaaga

SEQ ID NO: 287 - DNA, 19 bp, Homo sapiens
gagtctttgaagatagttt

SEQ ID NO: 288 - DNA, 19 bp, Homo sapiens
gtctttgaagatagtttta

SEQ ID NO: 289 - DNA, 19 bp, Homo sapiens
ctttgaagatagttttaat

SEQ ID NO: 290 - DNA, 19 bp, Homo sapiens
cagttatagcttattaggt

SEQ ID NO: 291 - DNA, 19 bp, Homo sapiens
gcttattaggtgttgaaga

SEQ ID NO: 292 - DNA, 19 bp, Homo sapiens
gctttcatagagagtttca

SEQ ID NO: 293 - DNA, 19 bp, Homo sapiens
catgcattggttagtcaaa

SEQ ID NO: 294 - DNA, 19 bp, Homo sapiens
cattgcttttgtttcttaa

SEQ ID NO: 295 - DNA, 19 bp, Homo sapiens
gcttttgtttcttaagaaa

SEQ ID NO: 296 - DNA, 19 bp, Homo sapiens
cttttgtttcttaagaaaa

SEQ ID NO: 297 - DNA, 19 bp, Homo sapiens
cttaagaaaacaaactctt

SEQ ID NO: 298 - DNA, 19 bp, Homo sapiens
gaaaacaaactctttttta

SEQ ID NO: 299 - DNA, 19 bp, Homo sapiens
caatgaagtgaaaaagttt

SEQ ID NO: 300 - DNA, 19 bp, Homo sapiens
gaagtgaaaaagttttaca

SEQ ID NO: 301 - DNA, 19 bp, Homo sapiens
ctcttaacactggttaaat

SEQ ID NO: 302 - DNA, 19 bp, Homo sapiens
cttaacactggttaaatta

SEQ ID NO: 303 - DNA, 19 bp, Homo sapiens
gttaaattaacattgcata

SEQ ID NO: 304 - DNA, 19 bp, Homo sapiens
gcataaacacttttcaagt

SEQ ID NO: 305 - DNA, 19 bp, Homo sapiens
caatccttttgataaattt

SEQ ID NO: 306 - DNA, 19 bp, Homo sapiens
gtgacttaggttctagata

SEQ ID NO: 307 - DNA, 19 bp, Homo sapiens
cttttaggactctgatttt

SEQ ID NO: 308 - DNA, 19 bp, Homo sapiens
catcacttactatccattt

SEQ ID NO: 309 - DNA, 19 bp, Homo sapiens
cacttactatccatttctt

SEQ ID NO: 310 - DNA, 19 bp, Homo sapiens
cttactatccatttcttca

SEQ ID NO: 311 - DNA, 19 bp, Homo sapiens
ctatccatttcttcatgtt

SEQ ID NO: 312 - DNA, 19 bp, Homo sapiens
ccatttcttcatgttaaaa

SEQ ID NO: 313 - DNA, 19 bp, Homo sapiens
gaagtcatctcaaactctt

SEQ ID NO: 314 - DNA, 19 bp, Homo sapiens
catctcaaactcttagttt

SEQ ID NO: 315 - DNA, 19 bp, Homo sapiens
ctcaaactcttagtttttt

SEQ ID NO: 316 - DNA, 19 bp, Homo sapiens
ctatgtaatttatattcca

SEQ ID NO: 317 - DNA, 19 bp, Homo sapiens
cataaggatacacttattt

SEQ ID NO: 318 - DNA, 19 bp, Homo sapiens
gcacaatctgtaaattttt

SEQ ID NO: 319 - DNA, 19 bp, Homo sapiens
ctatgttacaccatcttca

SEQ ID NO: 320 - RNA, 15 nt, Homo sapiens
caguuauagcuuauu

SEQ ID NO: 321 - RNA, 15 nt, Homo sapiens
ccuaguaggaaauaa

SEQ ID NO: 322 - RNA, 15 nt, Homo sapiens
agacccaguaugaaa

SEQ ID NO: 323 - RNA, 15 nt, Homo sapiens
gugccaagacauuaa

SEQ ID NO: 324 - RNA, 15 nt, Homo sapiens
cucuucuuccauauu

SEQ ID NO: 325 - RNA, 15 nt, Homo sapiens
aauggaaacuauuau

SEQ ID NO: 326 - RNA, 15 nt, Homo sapiens
guugauuacuucuua

SEQ ID NO: 327 - RNA, 15 nt, Homo sapiens
cuuagcaagaaguua

SEQ ID NO: 328 - RNA, 15 nt, Homo sapiens
cagcacaaucuguaa

SEQ ID NO: 329 - RNA, 15 nt, Homo sapiens
cuuuccacugcuauu

SEQ ID NO: 330 - RNA, 15 nt, Homo sapiens
ggugugaaacaaauu

SEQ ID NO: 331 - RNA, 15 nt, Homo sapiens
uacagcuaauucaga

SEQ ID NO: 332 - RNA, 15 nt, Homo sapiens
cuaauucagaaucau

SEQ ID NO: 333 - RNA, 15 nt, Homo sapiens
aauucagaaucauuu

SEQ ID NO: 334 - RNA, 15 nt, Homo sapiens
auaugauccaacaau

SEQ ID NO: 335 - RNA, 15 nt, Homo sapiens
gguaacaguaauaca

SEQ ID NO: 336 - RNA, 15 nt, Homo sapiens
caggacuuagcaaga

SEQ ID NO: 337 - RNA, 15 nt, Homo sapiens
ugugccagcucuaua

SEQ ID NO: 338 - RNA, 15 nt, Homo sapiens
cuauauuuacaugcu

SEQ ID NO: 339 - RNA, 15 nt, Homo sapiens
gucucuuggauauuc

SEQ ID NO: 340 - RNA, 15 nt, Homo sapiens
gggcuuucuuugugu

SEQ ID NO: 341 - RNA, 15 nt, Homo sapiens
gauaucuccaugaag

SEQ ID NO: 342 - RNA, 15 nt, Homo sapiens
cauuauagagaacaa

SEQ ID NO: 343 - RNA, 15 nt, Homo sapiens
cuguaauaucuuacu

SEQ ID NO: 344 - RNA, 15 nt, Homo sapiens
ugacgauacagcuaa

SEQ ID NO: 345 - RNA, 15 nt, Homo sapiens
gaugcuuugaacauc

SEQ ID NO: 346 - RNA, 15 nt, Homo sapiens
cccugaugaauguaa

SEQ ID NO: 347 - RNA, 15 nt, Homo sapiens
uauuugccauaaaua

SEQ ID NO: 348 - RNA, 15 nt, Homo sapiens
aaauaauacuaaauc

SEQ ID NO: 349 - RNA, 15 nt, Homo sapiens
aaucauuugaagaua

SEQ ID NO: 350 - RNA, 15 nt, Homo sapiens
gauauucaccauuau

SEQ ID NO: 351 - RNA, 15 nt, Homo sapiens
uuuaacaaaagauuu

SEQ ID NO: 352 - RNA, 15 nt, Homo sapiens
ccuaauauauacaua

SEQ ID NO: 353 - RNA, 15 nt, Homo sapiens
aaaagaaacugaaua

SEQ ID NO: 354 - RNA, 15 nt, Homo sapiens
agcacaaucuguaaa

SEQ ID NO: 355 - RNA, 15 nt, Homo sapiens
cuuucauaguauaac

SEQ ID NO: 356 - RNA, 15 nt, Homo sapiens
cuaguguggucugua

SEQ ID NO: 357 - RNA, 15 nt, Homo sapiens
guguggucuguaaua

SEQ ID NO: 358 - RNA, 15 nt, Homo sapiens
gacguauauuguauc

SEQ ID NO: 359 - RNA, 15 nt, Homo sapiens
cccaaguaggcauuc

SEQ ID NO: 360 - RNA, 15 nt, Homo sapiens
cgaauaugauccaac

SEQ ID NO: 361 - RNA, 15 nt, Homo sapiens
gcaaguaguaauuga

SEQ ID NO: 362 - RNA, 15 nt, Homo sapiens
uccugaugaugauuc

SEQ ID NO: 363 - RNA, 15 nt, Homo sapiens
gaccucaagugauuc

SEQ ID NO: 364 - RNA, 15 nt, Homo sapiens
ucccuaccuuccaca

SEQ ID NO: 365 - RNA, 15 nt, Homo sapiens
auuuccuuuucacau

SEQ ID NO: 366 - RNA, 15 nt, Homo sapiens
guauauuguaucauu

SEQ ID NO: 367 - RNA, 15 nt, Homo sapiens
cauuuccuuuucaca

SEQ ID NO: 368 - RNA, 15 nt, Homo sapiens
ggagaauucuagaaa

SEQ ID NO: 369 - RNA, 15 nt, Homo sapiens
cccaaguaggcauuc

SEQ ID NO: 370 - RNA, 15 nt, Homo sapiens
cgaauaugauccaac

SEQ ID NO: 371 - RNA, 15 nt, Homo sapiens
gcaaguaguaauuga

SEQ ID NO: 372 - RNA, 15 nt, Homo sapiens
uccugaugaugauuc

SEQ ID NO: 373 - RNA, 15 nt, Homo sapiens
cuguacuacuccuaa

SEQ ID NO: 374 - RNA, 15 nt, Homo sapiens
gaccucaagugauuc

SEQ ID NO: 375 - RNA, 15 nt, Homo sapiens
cuuagguagugcuag

SEQ ID NO: 376 - RNA, 15 nt, Homo sapiens
ucagacugcucuuuc

SEQ ID NO: 377 - RNA, 15 nt, Homo sapiens
ugcucuuucauagua

SEQ ID NO: 378 - RNA, 15 nt, Homo sapiens
gaugaauguaaaguu

SEQ ID NO: 379 - RNA, 15 nt, Homo sapiens
gucugauccauauuu

SEQ ID NO: 380 - RNA, 15 nt, Homo sapiens
gagcaaagaugguaa

SEQ ID NO: 381 - RNA, 15 nt, Homo sapiens
gaggugaaguuuaua

SEQ ID NO: 382 - RNA, 15 nt, Homo sapiens
aggguguuaagacuu

SEQ ID NO: 383 - RNA, 15 nt, Homo sapiens
ggcaucauguccuau

SEQ ID NO: 384 - RNA, 15 nt, Homo sapiens
ugaauguucccaagu

SEQ ID NO: 385 - RNA, 15 nt, Homo sapiens
aguaggaaauaaaug

SEQ ID NO: 386 - RNA, 15 nt, Homo sapiens
ugaacuaguucacag

SEQ ID NO: 387 - RNA, 15 nt, Homo sapiens
gaaaucuucaugcaa

SEQ ID NO: 388 - RNA, 15 nt, Homo sapiens
guggauaucuccaug

SEQ ID NO: 389 - RNA, 15 nt, Homo sapiens
agcaaguaguaauug

SEQ ID NO: 390 - RNA, 15 nt, Homo sapiens
acguauauuguauca

SEQ ID NO: 391 - RNA, 15 nt, Homo sapiens
uugacgauacagcua

SEQ ID NO: 392 - RNA, 15 nt, Homo sapiens
ggugacuuagguucu

SEQ ID NO: 393 - RNA, 15 nt, Homo sapiens
uaguucucuuaacac

SEQ ID NO: 394 - RNA, 15 nt, Homo sapiens
uaugucagauauuca

SEQ ID NO: 395 - RNA, 15 nt, Homo sapiens
ccuuugagcuuucau

SEQ ID NO: 396 - RNA, 15 nt, Homo sapiens
acaaggaaacuucua

SEQ ID NO: 397 - RNA, 15 nt, Homo sapiens
cgaucaagcuacuuu

SEQ ID NO: 398 - RNA, 15 nt, Homo sapiens
ugccaauuucuuacu

SEQ ID NO: 399 - RNA, 15 nt, Homo sapiens
gacaaaucaagagca

SEQ ID NO: 400 - RNA, 15 nt, Homo sapiens
aucucaaacucuuag

SEQ ID NO: 401 - RNA, 15 nt, Homo sapiens
gaugccuucuauaca

SEQ ID NO: 402 - RNA, 15 nt, Homo sapiens
guaugaauagacaga

SEQ ID NO: 403 - RNA, 15 nt, Homo sapiens
ugagucacaucagaa

SEQ ID NO: 404 - RNA, 15 nt, Homo sapiens
gucaccauugcacaa

SEQ ID NO: 405 - RNA, 15 nt, Homo sapiens
aagcucagcacaauc

SEQ ID NO: 406 - RNA, 15 nt, Homo sapiens
auuauuauagcaacc

SEQ ID NO: 407 - RNA, 15 nt, Homo sapiens
ggaagaaggugacuu

SEQ ID NO: 408 - RNA, 15 nt, Homo sapiens
cucugaagauguacc

SEQ ID NO: 409 - RNA, 15 nt, Homo sapiens
aguagcugggauuac

SEQ ID NO: 410 - RNA, 15 nt, Homo sapiens
gaguucuugaagaau

SEQ ID NO: 411 - RNA, 15 nt, Homo sapiens
gacgaauaugaucca

SEQ ID NO: 412 - RNA, 15 nt, Homo sapiens
uggauaucuccauga

SEQ ID NO: 413 - RNA, 15 nt, Homo sapiens
aaggaaacuucuaug

SEQ ID NO: 414 - RNA, 15 nt, Homo sapiens
uauagcagacguaua

SEQ ID NO: 415 - RNA, 15 nt, Homo sapiens
ucaagcuacuuuaug

SEQ ID NO: 416 - RNA, 15 nt, Homo sapiens
gguaugaauagacag

SEQ ID NO: 417 - RNA, 15 nt, Homo sapiens
uaauacauuccauug

SEQ ID NO: 418 - RNA, 15 nt, Homo sapiens
aaccuuugagcuuuc

SEQ ID NO: 419 - RNA, 15 nt, Homo sapiens
gaaaaugacugaaua

SEQ ID NO: 420 - RNA, 15 nt, Homo sapiens
aaaaugacugaauau

SEQ ID NO: 421 - RNA, 15 nt, Homo sapiens
aaugacugaauauaa

SEQ ID NO: 422 - RNA, 15 nt, Homo sapiens
uacagcuaauucaga

SEQ ID NO: 423 - RNA, 15 nt, Homo sapiens
cuaauucagaaucau

SEQ ID NO: 424 - RNA, 15 nt, Homo sapiens
aauucagaaucauuu

SEQ ID NO: 425 - RNA, 15 nt, Homo sapiens
ucauuuuguggacga

SEQ ID NO: 426 - RNA, 15 nt, Homo sapiens
auaugauccaacaau

SEQ ID NO: 427 - RNA, 15 nt, Homo sapiens
ccaacaauagaggau

SEQ ID NO: 428 - RNA, 15 nt, Homo sapiens
caauagaggauuccu

SEQ ID NO: 429 - RNA, 15 nt, Homo sapiens
uuccuacaggaagca

SEQ ID NO: 430 - RNA, 15 nt, Homo sapiens
acaggaagcaaguag

SEQ ID NO: 431 - RNA, 15 nt, Homo sapiens
caggaagcaaguagu

SEQ ID NO: 432 - RNA, 15 nt, Homo sapiens
gaagcaaguaguaau

SEQ ID NO: 433 - RNA, 15 nt, Homo sapiens
aguaguaauugaugg

SEQ ID NO: 434 - RNA, 15 nt, Homo sapiens
guaauugauggagaa

SEQ ID NO: 435 - RNA, 15 nt, Homo sapiens
ggagaaaccugucuc

SEQ ID NO: 436 - RNA, 15 nt, Homo sapiens
aaaccugucucuugg

SEQ ID NO: 437 - RNA, 15 nt, Homo sapiens
accugucucuuggau

SEQ ID NO: 438 - RNA, 15 nt, Homo sapiens
ucuuggauauucucg

SEQ ID NO: 439 - RNA, 15 nt, Homo sapiens
uuggauauucucgac

SEQ ID NO: 440 - RNA, 15 nt, Homo sapiens
ggauauucucgacac

SEQ ID NO: 441 - RNA, 15 nt, Homo sapiens
uauucucgacacagc

SEQ ID NO: 442 - RNA, 15 nt, Homo sapiens
gacacagcaggucaa

SEQ ID NO: 443 - RNA, 15 nt, Homo sapiens
ggucaagaggaguac

SEQ ID NO: 444 - RNA, 15 nt, Homo sapiens
gaggaguacagugca

SEQ ID NO: 445 - RNA, 15 nt, Homo sapiens
gaguacagugcaaug

SEQ ID NO: 446 - RNA, 14 nt, Homo sapiens
ugagggaccaguac

SEQ ID NO: 447 - RNA, 15 nt, Homo sapiens
ccaguacaugaggac

SEQ ID NO: 448 - RNA, 15 nt, Homo sapiens
agggcuuucuuugug

SEQ ID NO: 449 - RNA, 15 nt, Homo sapiens
ggcuuucuuugugua

SEQ ID NO: 450 - RNA, 15 nt, Homo sapiens
cuuucuuuguguauu

SEQ ID NO: 451 - RNA, 15 nt, Homo sapiens
uguguauuugccaua

SEQ ID NO: 452 - RNA, 14 nt, Homo sapiens
uuugccauaaauaa

SEQ ID NO: 453 - RNA, 15 nt, Homo sapiens
aaucauuugaagaua

SEQ ID NO: 454 - RNA, 15 nt, Homo sapiens
gauauucaccauuau

SEQ ID NO: 455 - RNA, 15 nt, Homo sapiens
uuauagagaacaaau

SEQ ID NO: 456 - RNA, 15 nt, Homo sapiens
uauagagaacaaauu

SEQ ID NO: 457 - RNA, 15 nt, Homo sapiens
aacaaauuaaaagag

SEQ ID NO: 458 - RNA, 15 nt, Homo sapiens
caaauuaaaagaguu

SEQ ID NO: 459 - RNA, 15 nt, Homo sapiens
uuaaggacucugaag

SEQ ID NO: 460 - RNA, 15 nt, Homo sapiens
aaggacucugaagau

SEQ ID NO: 461 - RNA, 15 nt, Homo sapiens
cucugaagauguacc

SEQ ID NO: 462 - RNA, 15 nt, Homo sapiens
ucugaagauguaccu

SEQ ID NO: 463 - RNA, 15 nt, Homo sapiens
aagauguaccuaugg

SEQ ID NO: 464 - RNA, 15 nt, Homo sapiens
augguccuaguagga

SEQ ID NO: 465 - RNA, 15 nt, Homo sapiens
ugguccuaguaggaa

SEQ ID NO: 466 - RNA, 15 nt, Homo sapiens
cuaguaggaaauaaa

SEQ ID NO: 467 - RNA, 15 nt, Homo sapiens
ggaaauaaaugugau

SEQ ID NO: 468 - RNA, 15 nt, Homo sapiens
ucuagaacaguagac

SEQ ID NO: 469 - RNA, 15 nt, Homo sapiens
gaacaguagacacaa

SEQ ID NO: 470 - RNA, 15 nt, Homo sapiens
caguagacacaaaac

SEQ ID NO: 471 - RNA, 15 nt, Homo sapiens
aacaggcucaggacu

SEQ ID NO: 472 - RNA, 15 nt, Homo sapiens
gcucaggacuuagca

SEQ ID NO: 473 - RNA, 15 nt, Homo sapiens
ucaggacuuagcaag

SEQ ID NO: 474 - RNA, 15 nt, Homo sapiens
gacuuagcaagaagu

SEQ ID NO: 475 - RNA, 15 nt, Homo sapiens
agcaagaaguuaugg

SEQ ID NO: 476 - RNA, 15 nt, Homo sapiens
agaaguuauggaauu

SEQ ID NO: 477 - RNA, 15 nt, Homo sapiens
guuauggaauuccuu

SEQ ID NO: 478 - RNA, 15 nt, Homo sapiens
auggaauuccuuuua

SEQ ID NO: 479 - RNA, 15 nt, Homo sapiens
auuccuuuuauugaa

SEQ ID NO: 480 - RNA, 15 nt, Homo sapiens
acaucagcaaagaca

SEQ ID NO: 481 - RNA, 15 nt, Homo sapiens
cagcaaagacaagac

SEQ ID NO: 482 - RNA, 15 nt, Homo sapiens
aagacaagacagggu

SEQ ID NO: 483 - RNA, 15 nt, Homo sapiens
agacaagacagggug

SEQ ID NO: 484 - RNA, 15 nt, Homo sapiens
aagacaggguguuga

SEQ ID NO: 485 - RNA, 15 nt, Homo sapiens
gacaggguguugaug

SEQ ID NO: 486 - RNA, 15 nt, Homo sapiens
gguguugaugaugcc

SEQ ID NO: 487 - RNA, 15 nt, Homo sapiens
guugaugaugccuuc

SEQ ID NO: 488 - RNA, 15 nt, Homo sapiens
uugaugaugccuucu

SEQ ID NO: 489 - RNA, 15 nt, Homo sapiens
gaugaugccuucuau

SEQ ID NO: 490 - RNA, 15 nt, Homo sapiens
augccuucuauacau

SEQ ID NO: 491 - RNA, 15 nt, Homo sapiens
gccuucuauacauua

SEQ ID NO: 492 - RNA, 15 nt, Homo sapiens
uucuauacauuaguu

SEQ ID NO: 493 - RNA, 15 nt, Homo sapiens
cuauacauuaguucg

SEQ ID NO: 494 - RNA, 15 nt, Homo sapiens
uacauuaguucgaga

SEQ ID NO: 495 - RNA, 15 nt, Homo sapiens
gaaauucgaaaacau

SEQ ID NO: 496 - RNA, 15 nt, Homo sapiens
aaauucgaaaacaua

SEQ ID NO: 497 - RNA, 15 nt, Homo sapiens
aaacauaaagaaaag

SEQ ID NO: 498 - RNA, 15 nt, Homo sapiens
aacauaaagaaaaga

SEQ ID NO: 499 - RNA, 15 nt, Homo sapiens
aaagaaaagaugagc

SEQ ID NO: 500 - RNA, 15 nt, Homo sapiens
aagaugguaaaaaga

SEQ ID NO: 501 - RNA, 15 nt, Homo sapiens
agaugguaaaaagaa

SEQ ID NO: 502 - RNA, 15 nt, Homo sapiens
gguaaaaagaagaaa

SEQ ID NO: 503 - RNA, 15 nt, Homo sapiens
aaaaagaagaaaaag

SEQ ID NO: 504 - RNA, 15 nt, Homo sapiens
aaaagaagaaaaaga

SEQ ID NO: 505 - RNA, 15 nt, Homo sapiens
gaaaaagaagucaaa

SEQ ID NO: 506 - RNA, 15 nt, Homo sapiens
aaagaagucaaagac

SEQ ID NO: 507 - RNA, 15 nt, Homo sapiens
gucaaagacaaagug

SEQ ID NO: 508 - RNA, 15 nt, Homo sapiens
aaagacaaagugugu

SEQ ID NO: 509 - RNA, 15 nt, Homo sapiens
agacaaaguguguaa

SEQ ID NO: 510 - RNA, 15 nt, Homo sapiens
aaaguguguaauuau

SEQ ID NO: 511 - RNA, 15 nt, Homo sapiens
aguguguaauuaugu

SEQ ID NO: 512 - RNA, 15 nt, Homo sapiens
uuuguacuuuuuucu

SEQ ID NO: 513 - RNA, 15 nt, Homo sapiens
cuuuuuucuuaaggc

SEQ ID NO: 514 - RNA, 15 nt, Homo sapiens
uuuucuuaaggcaua

SEQ ID NO: 515 - RNA, 15 nt, Homo sapiens
aaggcauacuaguac

SEQ ID NO: 516 - RNA, 15 nt, Homo sapiens
acuaguacaaguggu

SEQ ID NO: 517 - RNA, 15 nt, Homo sapiens
guacaagugguaauu

SEQ ID NO: 518 - RNA, 15 nt, Homo sapiens
caagugguaauuuuu

SEQ ID NO: 519 - RNA, 15 nt, Homo sapiens
guaauuuuuguacau

SEQ ID NO: 520 - RNA, 15 nt, Homo sapiens
auuuuuguacauuac

SEQ ID NO: 521 - RNA, 15 nt, Homo sapiens
cauuacacuaaauua

SEQ ID NO: 522 - RNA, 15 nt, Homo sapiens
uacacuaaauuauua

SEQ ID NO: 523 - RNA, 15 nt, Homo sapiens
aauuauuagcauuug

SEQ ID NO: 524 - RNA, 15 nt, Homo sapiens
uuuguuuuagcauua

SEQ ID NO: 525 - RNA, 15 nt, Homo sapiens
uuagcauuaccuaau

SEQ ID NO: 526 - RNA, 15 nt, Homo sapiens
gcuccaugcagacug

SEQ ID NO: 527 - RNA, 15 nt, Homo sapiens
cuccaugcagacugu

SEQ ID NO: 528 - RNA, 15 nt, Homo sapiens
ccaugcagacuguua

SEQ ID NO: 529 - RNA, 15 nt, Homo sapiens
ugcagacuguuagcu

SEQ ID NO: 530 - RNA, 16 nt, Homo sapiens
uguuagcuuuuaccuu

SEQ ID NO: 531 - RNA, 15 nt, Homo sapiens
agcuuuuaccuuaaa

SEQ ID NO: 532 - RNA, 15 nt, Homo sapiens
uuuaccuuaaaugcu

SEQ ID NO: 533 - RNA, 15 nt, Homo sapiens
uuaccuuaaaugcuu

SEQ ID NO: 534 - RNA, 15 nt, Homo sapiens
uuuuuuuccucuaag

SEQ ID NO: 535 - RNA, 15 nt, Homo sapiens
guauucccagaguuu

SEQ ID NO: 536 - RNA, 15 nt, Homo sapiens
aguuuugguuuuuga

SEQ ID NO: 537 - RNA, 15 nt, Homo sapiens
uuuugguuuuugaac

SEQ ID NO: 538 - RNA, 15 nt, Homo sapiens
gcaaugccugugaaa

SEQ ID NO: 539 - RNA, 15 nt, Homo sapiens
ugaaaaagaaacuga

SEQ ID NO: 540 - RNA, 15 nt, Homo sapiens
aaaaagaaacugaau

SEQ ID NO: 541 - RNA, 15 nt, Homo sapiens
aauaccuaagauuuc

SEQ ID NO: 542 - RNA, 15 nt, Homo sapiens
uaccuaagauuucug

SEQ ID NO: 543 - RNA, 15 nt, Homo sapiens
uugauuacuucuuau

SEQ ID NO: 544 - RNA, 15 nt, Homo sapiens
gauuacuucuuauuu

SEQ ID NO: 545 - RNA, 15 nt, Homo sapiens
uacuucuuauuuuuc

SEQ ID NO: 546 - RNA, 15 nt, Homo sapiens
auuuuucuuaccaau

SEQ ID NO: 547 - RNA, 15 nt, Homo sapiens
accaauugugaaugu

SEQ ID NO: 548 - RNA, 15 nt, Homo sapiens
uguuggugugaaaca

SEQ ID NO: 549 - RNA, 15 nt, Homo sapiens
ugaaacaaauuaaug

SEQ ID NO: 550 - RNA, 15 nt, Homo sapiens
auucuguguuuuauc

SEQ ID NO: 551 - RNA, 15 nt, Homo sapiens
uucuguguuuuaucu

SEQ ID NO: 552 - RNA, 15 nt, Homo sapiens
aaauggauuaauuac

SEQ ID NO: 553 - RNA, 15 nt, Homo sapiens
cuaauugguuuuuac

SEQ ID NO: 554 - RNA, 15 nt, Homo sapiens
auugguuuuuacuga

SEQ ID NO: 555 - RNA, 15 nt, Homo sapiens
uuuacugaaacauug

SEQ ID NO: 556 - RNA, 15 nt, Homo sapiens
ucauguccuauaguu

SEQ ID NO: 557 - RNA, 15 nt, Homo sapiens
cacaaagguuuuguc

SEQ ID NO: 558 - RNA, 15 nt, Homo sapiens
auuuccuuuucacau

SEQ ID NO: 559 - RNA, 15 nt, Homo sapiens
uuuccuuuucacauu

SEQ ID NO: 560 - RNA, 15 nt, Homo sapiens
caugcagacuguuag

SEQ ID NO: 561 - RNA, 15 nt, Homo sapiens
gcagacuguuagcuu

SEQ ID NO: 562 - RNA, 15 nt, Homo sapiens
gacuguuagcuuuua

SEQ ID NO: 563 - RNA, 15 nt, Homo sapiens
uuagcuuuuaccuua

SEQ ID NO: 564 - RNA, 15 nt, Homo sapiens
uaaaugcuuauuuua

SEQ ID NO: 565 - RNA, 15 nt, Homo sapiens
aguggaaguuuuuuu

SEQ ID NO: 566 - RNA, 15 nt, Homo sapiens
guggaaguuuuuuuu

SEQ ID NO: 567 - RNA, 15 nt, Homo sapiens
cuaagugccaguauu

SEQ ID NO: 568 - RNA, 15 nt, Homo sapiens
agugccaguauuccc

SEQ ID NO: 569 - RNA, 15 nt, Homo sapiens
ccaguauucccagag

SEQ ID NO: 570 - RNA, 15 nt, Homo sapiens
aguauucccagaguu

SEQ ID NO: 571 - RNA, 15 nt, Homo sapiens
guauucccagaguuu

SEQ ID NO: 572 - RNA, 15 nt, Homo sapiens
uauucccagaguuuu

SEQ ID NO: 573 - RNA, 15 nt, Homo sapiens
uucccagaguuuugg

SEQ ID NO: 574 - RNA, 15 nt, Homo sapiens
agaguuuugguuuuu

SEQ ID NO: 575 - RNA, 15 nt, Homo sapiens
gaguuuugguuuuug

SEQ ID NO: 576 - RNA, 15 nt, Homo sapiens
guuuugguuuuugaa

SEQ ID NO: 577 - RNA, 15 nt, Homo sapiens
uugguuuuugaacua

SEQ ID NO: 578 - RNA, 15 nt, Homo sapiens
uuuugaacuagcaau

SEQ ID NO: 579 - RNA, 15 nt, Homo sapiens
cuagcaaugccugug

SEQ ID NO: 580 - RNA, 15 nt, Homo sapiens
augccugugaaaaag

SEQ ID NO: 581 - RNA, 15 nt, Homo sapiens
ugccugugaaaaaga

SEQ ID NO: 582 - RNA, 15 nt, Homo sapiens
ugugaaaaagaaacu

SEQ ID NO: 583 - RNA, 15 nt, Homo sapiens
gugaaaaagaaacug

SEQ ID NO: 584 - RNA, 15 nt, Homo sapiens
aaaagaaacugaaua

SEQ ID NO: 585 - RNA, 15 nt, Homo sapiens
acugaauaccuaaga

SEQ ID NO: 586 - RNA, 15 nt, Homo sapiens
ucuugggguuuuugg

SEQ ID NO: 587 - RNA, 15 nt, Homo sapiens
uugggguuuuuggug

SEQ ID NO: 588 - RNA, 15 nt, Homo sapiens
gggguuuuuggugca

SEQ ID NO: 589 - RNA, 15 nt, Homo sapiens
ugcaguguuuuuggu

SEQ ID NO: 590 - RNA, 15 nt, Homo sapiens
uuuggugcaugcagu

SEQ ID NO: 591 - RNA, 15 nt, Homo sapiens
uuucacauuagauaa

SEQ ID NO: 592 - RNA, 15 nt, Homo sapiens
uucacauuagauaaa

SEQ ID NO: 593 - RNA, 15 nt, Homo sapiens
ugaaauggggauuau

SEQ ID NO: 594 - RNA, 15 nt, Homo sapiens
uuuuggggcuauauu

SEQ ID NO: 595 - RNA, 15 nt, Homo sapiens
uuuggggcuauauuu

SEQ ID NO: 596 - RNA, 15 nt, Homo sapiens
aagauuuuaacaagu

SEQ ID NO: 597 - RNA, 15 nt, Homo sapiens
guauaaaaaauucuc

SEQ ID NO: 598 - RNA, 15 nt, Homo sapiens
aggaauuaaauguag

SEQ ID NO: 599 - RNA, 15 nt, Homo sapiens
cuuucauaguauaac

SEQ ID NO: 600 - RNA, 15 nt, Homo sapiens
uuucauaguauaacu

SEQ ID NO: 601 - RNA, 15 nt, Homo sapiens
ucauaguauaacuuu

SEQ ID NO: 602 - RNA, 15 nt, Homo sapiens
uaaaucuuuucuuca

SEQ ID NO: 603 - RNA, 15 nt, Homo sapiens
caacuugagucuuug

SEQ ID NO: 604 - RNA, 15 nt, Homo sapiens
cuugagucuuugaag

SEQ ID NO: 605 - RNA, 15 nt, Homo sapiens
ucuuugaagauaguu

SEQ ID NO: 606 - RNA, 15 nt, Homo sapiens
uuugaagauaguuuu

SEQ ID NO: 607 - RNA, 15 nt, Homo sapiens
ugaagauaguuuuaa

SEQ ID NO: 608 - RNA, 15 nt, Homo sapiens
uuauagcuuauuagg

SEQ ID NO: 609 - RNA, 15 nt, Homo sapiens
uauuagguguugaag

SEQ ID NO: 610 - RNA, 15 nt, Homo sapiens
uucauagagaguuuc

SEQ ID NO: 611 - RNA, 15 nt, Homo sapiens
gcauugguuagucaa

SEQ ID NO: 612 - RNA, 15 nt, Homo sapiens
ugcuuuuguuucuua

SEQ ID NO: 613 - RNA, 15 nt, Homo sapiens
uuuguuucuuaagaa

SEQ ID NO: 614 - RNA, 15 nt, Homo sapiens
uuguuucuuaagaaa

SEQ ID NO: 615 - RNA, 15 nt, Homo sapiens
aagaaaacaaacucu

SEQ ID NO: 616 - RNA, 15 nt, Homo sapiens
aacaaacucuuuuuu

SEQ ID NO: 617 - RNA, 15 nt, Homo sapiens
ugaagugaaaaaguu

SEQ ID NO: 618 - RNA, 15 nt, Homo sapiens
gugaaaaaguuuuac

SEQ ID NO: 619 - RNA, 15 nt, Homo sapiens
uuaacacugguuaaa

SEQ ID NO: 620 - RNA, 15 nt, Homo sapiens
aacacugguuaaauu

SEQ ID NO: 621 - RNA, 15 nt, Homo sapiens
aaauuaacauugcau

SEQ ID NO: 622 - RNA, 15 nt, Homo sapiens
uaaacacuuuucaag

SEQ ID NO: 623 - RNA, 15 nt, Homo sapiens
uccuuuugauaaauu

SEQ ID NO: 624 - RNA, 15 nt, Homo sapiens
acuuagguucuagau

SEQ ID NO: 625 - RNA, 15 nt, Homo sapiens
uuaggacucugauuu

SEQ ID NO: 626 - RNA, 15 nt, Homo sapiens
cacuuacuauccauu

SEQ ID NO: 627 - RNA, 15 nt, Homo sapiens
uuacuauccauuucu

SEQ ID NO: 628 - RNA, 15 nt, Homo sapiens
acuauccauuucuuc

SEQ ID NO: 629 - RNA, 15 nt, Homo sapiens
uccauuucuucaugu

SEQ ID NO: 630 - RNA, 15 nt, Homo sapiens
uuucuucauguuaaa

SEQ ID NO: 631 - RNA, 15 nt, Homo sapiens
gucaucucaaacucu

SEQ ID NO: 632 - RNA, 15 nt, Homo sapiens
cucaaacucuuaguu

SEQ ID NO: 633 - RNA, 15 nt, Homo sapiens
aaacucuuaguuuuu

SEQ ID NO: 634 - RNA, 15 nt, Homo sapiens
uguaauuuauauucc

SEQ ID NO: 635 - RNA, 15 nt, Homo sapiens
aaggauacacuuauu

SEQ ID NO: 636 - RNA, 15 nt, Homo sapiens
caaucuguaaauuuu

SEQ ID NO: 637 - RNA, 15 nt, Homo sapiens
uguuacaccaucuuc

SEQ ID NO: 638 - RNA, 21 nt, Homo sapiens
aauaauaagcuauaacuggcc

SEQ ID NO: 639 - RNA, 21 nt, Homo sapiens
aauuuauuuccuacuaggacc

SEQ ID NO: 640 - RNA, 21 nt, Homo sapiens
aaauuucauacugggucugcc

SEQ ID NO: 641 - RNA, 21 nt, Homo sapiens
aaauuaaugucuuggcacacc

SEQ ID NO: 642 - RNA, 21 nt, Homo sapiens
aauaauauggaagaagagucc

SEQ ID NO: 643 - RNA, 21 nt, Homo sapiens
aauauaauaguuuccauugcc

SEQ ID NO: 644 - RNA, 21 nt, Homo sapiens
aaauaagaaguaaucaacugc

SEQ ID NO: 645 - RNA, 21 nt, Homo sapiens
aaauaacuucuugcuaagucc

SEQ ID NO: 646 - RNA, 21 nt, Homo sapiens
aauuuacagauugugcugagc

SEQ ID NO: 647 - RNA, 21 nt, Homo sapiens
aauaauagcaguggaaaggag

SEQ ID NO: 648 - RNA, 21 nt, Homo sapiens
aauaauuuguuucacaccaac

SEQ ID NO: 649 - RNA, 21 nt, Homo sapiens
aauucugaauuagcuguaucg

SEQ ID NO: 650 - RNA, 21 nt, Homo sapiens
aaaaugauucugaauuagcug

SEQ ID NO: 651 - RNA, 21 nt, Homo sapiens
aaaaaaugauucugaauuagc

SEQ ID NO: 652 - RNA, 21 nt, Homo sapiens
aauauuguuggaucauauucg

SEQ ID NO: 653 - RNA, 21 nt, Homo sapiens
aaauguauuacuguuaccagg

SEQ ID NO: 654 - RNA, 21 nt, Homo sapiens
aauucuugcuaaguccugagc

SEQ ID NO: 655 - RNA, 21 nt, Homo sapiens
aauuauagagcuggcacagag

SEQ ID NO: 656 - RNA, 21 nt, Homo sapiens
aauagcauguaaauauagccc

SEQ ID NO: 657 - RNA, 21 nt, Homo sapiens
aaagaauauccaagagacagg

SEQ ID NO: 658 - RNA, 21 nt, Homo sapiens
aauacacaaagaaagcccucc

SEQ ID NO: 659 - RNA, 21 nt, Homo sapiens
aaacuucauggagauauccac

SEQ ID NO: 660 - RNA, 21 nt, Homo sapiens
aauuuguucucuauaauggug

SEQ ID NO: 661 - RNA, 21 nt, Homo sapiens
aauaguaagauauuacagacc

SEQ ID NO: 662 - RNA, 21 nt, Homo sapiens
aaauuagcuguaucgucaagg

SEQ ID NO: 663 - RNA, 21 nt, Homo sapiens
aaagauguucaaagcaucagc

SEQ ID NO: 664 - RNA, 21 nt, Homo sapiens
aauuuacauucaucagggaug

SEQ ID NO: 665 - RNA, 21 nt, Homo sapiens
aauuauuuauggcaaauacac

SEQ ID NO: 666 - RNA, 21 nt, Homo sapiens
aaugauuuaguauuauuuaug

SEQ ID NO: 667 - RNA, 21 nt, Homo sapiens
aaauaucuucaaaugauuuag

SEQ ID NO: 668 - RNA, 21 nt, Homo sapiens
aauauaauggugaauaucuuc

SEQ ID NO: 669 - RNA, 21 nt, Homo sapiens
aacaaaucuuuuguuaaacca

SEQ ID NO: 670 - RNA, 21 nt, Homo sapiens
aacuauguauauauuaggaca

SEQ ID NO: 671 - RNA, 21 nt, Homo sapiens
aaguauucaguuucuuuuuca

SEQ ID NO: 672 - RNA, 21 nt, Homo sapiens
aaauuuacagauugugcugag

SEQ ID NO: 673 - RNA, 21 nt, Homo sapiens
aaaguuauacuaugaaagagc

SEQ ID NO: 674 - RNA, 21 nt, Homo sapiens
aauuacagaccacacuagcac

SEQ ID NO: 675 - RNA, 21 nt, Homo sapiens
aaauauuacagaccacacuag

SEQ ID NO: 676 - RNA, 21 nt, Homo sapiens
aaugauacaauauacgucugc

SEQ ID NO: 677 - RNA, 21 nt, Homo sapiens
aaagaaugccuacuugggaac

SEQ ID NO: 678 - RNA, 21 nt, Homo sapiens
aauguuggaucauauucgucc

SEQ ID NO: 679 - RNA, 21 nt, Homo sapiens
aaaucaauuacuacuugcuuc

SEQ ID NO: 680 - RNA, 21 nt, Homo sapiens
aaagaaucaucaucaggaagc

SEQ ID NO: 681 - RNA, 21 nt, Homo sapiens
aaugaaucacuugaggucagg

SEQ ID NO: 682 - RNA, 21 nt, Homo sapiens
aaauguggaagguagggaggc

SEQ ID NO: 683 - RNA, 21 nt, Homo sapiens
aaaaugugaaaaggaaauggc

SEQ ID NO: 684 - RNA, 21 nt, Homo sapiens
aaaaaugauacaauauacguc

SEQ ID NO: 685 - RNA, 21 nt, Homo sapiens
aaaugugaaaaggaaauggcc

SEQ ID NO: 686 - RNA, 21 nt, Homo sapiens
aaauuucuagaauucuccccc

SEQ ID NO: 687 - RNA, 21 nt, Homo sapiens
aaagaaugccuacuugggaac

SEQ ID NO: 688 - RNA, 21 nt, Homo sapiens
aauguuggaucauauucgucc

SEQ ID NO: 689 - RNA, 21 nt, Homo sapiens
aaaucaauuacuacuugcuuc

SEQ ID NO: 690 - RNA, 21 nt, Homo sapiens
aaagaaucaucaucaggaagc

SEQ ID NO: 691 - RNA, 21 nt, Homo sapiens
aaauuaggaguaguacaguuc

SEQ ID NO: 692 - RNA, 21 nt, Homo sapiens
aaugaaucacuugaggucagg

SEQ ID NO: 693 - RNA, 21 nt, Homo sapiens
aaacuagcacuaccuaaggac

SEQ ID NO: 694 - RNA, 21 nt, Homo sapiens
aaugaaagagcagucugacac

SEQ ID NO: 695 - RNA, 21 nt, Homo sapiens
aaauacuaugaaagagcaguc

SEQ ID NO: 696 - RNA, 21 nt, Homo sapiens
aauaacuuuacauucaucagg

SEQ ID NO: 697 - RNA, 21 nt, Homo sapiens
aauaaauauggaucagacuug

SEQ ID NO: 698 - RNA, 21 nt, Homo sapiens
aauuuaccaucuuugcucauc

SEQ ID NO: 699 - RNA, 21 nt, Homo sapiens
aaauauaaacuucaccucuug

SEQ ID NO: 700 - RNA, 21 nt, Homo sapiens
aauaagucuuaacacccuacc

SEQ ID NO: 701 - RNA, 21 nt, Homo sapiens
aauauaggacaugaugccuag

SEQ ID NO: 702 - RNA, 21 nt, Homo sapiens
aauacuugggaacauucacuc

SEQ ID NO: 703 - RNA, 21 nt, Homo sapiens
aaacauuuauuuccuacuagg

SEQ ID NO: 704 - RNA, 21 nt, Homo sapiens
aaucugugaacuaguucaggc

SEQ ID NO: 705 - RNA, 21 nt, Homo sapiens
aaauugcaugaagauuucugg

SEQ ID NO: 706 - RNA, 21 nt, Homo sapiens
aaucauggagauauccacagc

SEQ ID NO: 707 - RNA, 21 nt, Homo sapiens
aaucaauuacuacuugcuucc

SEQ ID NO: 708 - RNA, 21 nt, Homo sapiens
aaaugauacaauauacgucug

SEQ ID NO: 709 - RNA, 21 nt, Homo sapiens
aauuagcuguaucgucaaggc

SEQ ID NO: 710 - RNA, 21 nt, Homo sapiens
aauagaaccuaagucaccuuc

SEQ ID NO: 711 - RNA, 21 nt, Homo sapiens
aaaguguuaagagaacuagcc

SEQ ID NO: 712 - RNA, 21 nt, Homo sapiens
aaaugaauaucugacauacac

SEQ ID NO: 713 - RNA, 21 nt, Homo sapiens
aauaugaaagcucaaagguuc

SEQ ID NO: 714 - RNA, 21 nt, Homo sapiens
aaauagaaguuuccuugucug

SEQ ID NO: 715 - RNA, 21 nt, Homo sapiens
aauaaaguagcuugaucgaag

SEQ ID NO: 716 - RNA, 21 nt, Homo sapiens
aauaguaagaaauuggcacuc

SEQ ID NO: 717 - RNA, 21 nt, Homo sapiens
aaaugcucuugauuugucagc

SEQ ID NO: 718 - RNA, 21 nt, Homo sapiens
aaacuaagaguuugagaugac

SEQ ID NO: 719 - RNA, 21 nt, Homo sapiens
aaauguauagaaggcaucauc

SEQ ID NO: 720 - RNA, 21 nt, Homo sapiens
aauucugucuauucauaccag

SEQ ID NO: 721 - RNA, 21 nt, Homo sapiens
aauuucugaugugacucagug

SEQ ID NO: 722 - RNA, 21 nt, Homo sapiens
aaauugugcaauggugacaac

SEQ ID NO: 723 - RNA, 21 nt, Homo sapiens
aaagauugugcugagcuugac

SEQ ID NO: 724 - RNA, 21 nt, Homo sapiens
aaugguugcuauaauaauccc

SEQ ID NO: 725 - RNA, 21 nt, Homo sapiens
aauaagucaccuucuuccuag

SEQ ID NO: 726 - RNA, 21 nt, Homo sapiens
aaagguacaucuucagagucc

SEQ ID NO: 727 - RNA, 21 nt, Homo sapiens
aauguaaucccagcuacucag

SEQ ID NO: 728 - RNA, 21 nt, Homo sapiens
aauauucuucaagaacucaug

SEQ ID NO: 729 - RNA, 21 nt, Homo sapiens
aauuggaucauauucguccac

SEQ ID NO: 730 - RNA, 21 nt, Homo sapiens
aauucauggagauauccacag

SEQ ID NO: 731 - RNA, 21 nt, Homo sapiens
aaacauagaaguuuccuuguc

SEQ ID NO: 732 - RNA, 21 nt, Homo sapiens
aaauauacgucugcuauauuc

SEQ ID NO: 733 - RNA, 21 nt, Homo sapiens
aaacauaaaguagcuugaucg

SEQ ID NO: 734 - RNA, 21 nt, Homo sapiens
aaucugucuauucauaccagg

SEQ ID NO: 735 - RNA, 21 nt, Homo sapiens
aaacaauggaauguauuacug

SEQ ID NO: 736 - RNA, 21 nt, Homo sapiens
aaugaaagcucaaagguucac

SEQ ID NO: 737 - RNA, 21 nt, Homo sapiens
aaauauucagucauuuucagc

SEQ ID NO: 738 - RNA, 21 nt, Homo sapiens
aauauauucagucauuuucag

SEQ ID NO: 739 - RNA, 21 nt, Homo sapiens
aauuuauauucagucauuuuc

SEQ ID NO: 740 - RNA, 21 nt, Homo sapiens
aauucugaauuagcuguaucg

SEQ ID NO: 741 - RNA, 21 nt, Homo sapiens
aaaaugauucugaauuagcug

SEQ ID NO: 742 - RNA, 21 nt, Homo sapiens
aaaaaaugauucugaauuagc

SEQ ID NO: 743 - RNA, 21 nt, Homo sapiens
aauucguccacaaaaugauuc

SEQ ID NO: 744 - RNA, 21 nt, Homo sapiens
aauauuguuggaucauauucg

SEQ ID NO: 745 - RNA, 21 nt, Homo sapiens
aaaauccucuauuguuggauc

SEQ ID NO: 746 - RNA, 21 nt, Homo sapiens
aauaggaauccucuauuguug

SEQ ID NO: 747 - RNA, 21 nt, Homo sapiens
aauugcuuccuguaggaaucc

SEQ ID NO: 748 - RNA, 21 nt, Homo sapiens
aaacuacuugcuuccuguagg

SEQ ID NO: 749 - RNA, 21 nt, Homo sapiens
aauacuacuugcuuccuguag

SEQ ID NO: 750 - RNA, 21 nt, Homo sapiens
aaaauuacuacuugcuuccug

SEQ ID NO: 751 - RNA, 21 nt, Homo sapiens
aauccaucaauuacuacuugc

SEQ ID NO: 752 - RNA, 21 nt, Homo sapiens
aauuucuccaucaauuacuac

SEQ ID NO: 753 - RNA, 21 nt, Homo sapiens
aaagagacagguuucuccauc

SEQ ID NO: 754 - RNA, 21 nt, Homo sapiens
aauccaagagacagguuucuc

SEQ ID NO: 755 - RNA, 21 nt, Homo sapiens
aauauccaagagacagguuuc

SEQ ID NO: 756 - RNA, 21 nt, Homo sapiens
aaucgagaauauccaagagac

SEQ ID NO: 757 - RNA, 21 nt, Homo sapiens
aaugucgagaauauccaagag

SEQ ID NO: 758 - RNA, 21 nt, Homo sapiens
aaugugucgagaauauccaag

SEQ ID NO: 759 - RNA, 21 nt, Homo sapiens
aaugcugugucgagaauaucc

SEQ ID NO: 760 - RNA, 21 nt, Homo sapiens
aacuugaccugcugugucgag

SEQ ID NO: 761 - RNA, 21 nt, Homo sapiens
aauguacuccucuugaccugc

SEQ ID NO: 762 - RNA, 21 nt, Homo sapiens
aauugcacuguacuccucuug

SEQ ID NO: 763 - RNA, 21 nt, Homo sapiens
aaucauugcacuguacuccuc

SEQ ID NO: 764 - RNA, 20 nt, Homo sapiens
aauguacuggucccucauug

SEQ ID NO: 765 - RNA, 21 nt, Homo sapiens
aaaguccucauguacuggucc

SEQ ID NO: 766 - RNA, 21 nt, Homo sapiens
aaacacaaagaaagcccuccc

SEQ ID NO: 767 - RNA, 21 nt, Homo sapiens
aaauacacaaagaaagcccuc

SEQ ID NO: 768 - RNA, 21 nt, Homo sapiens
aaaaauacacaaagaaagccc

SEQ ID NO: 769 - RNA, 21 nt, Homo sapiens
aauuauggcaaauacacaaag

SEQ ID NO: 770 - RNA, 20 nt, Homo sapiens
aaauuauuuauggcaaauac

SEQ ID NO: 771 - RNA, 21 nt, Homo sapiens
aaauaucuucaaaugauuuag

SEQ ID NO: 772 - RNA, 21 nt, Homo sapiens
aauauaauggugaauaucuuc

SEQ ID NO: 773 - RNA, 21 nt, Homo sapiens
aaaauuuguucucuauaaugg

SEQ ID NO: 774 - RNA, 21 nt, Homo sapiens
aauaauuuguucucuauaaug

SEQ ID NO: 775 - RNA, 21 nt, Homo sapiens
aaacucuuuuaauuuguucuc

SEQ ID NO: 776 - RNA, 21 nt, Homo sapiens
aauaacucuuuuaauuuguuc

SEQ ID NO: 777 - RNA, 21 nt, Homo sapiens
aaucuucagaguccuuaacuc

SEQ ID NO: 778 - RNA, 21 nt, Homo sapiens
aacaucuucagaguccuuaac

SEQ ID NO: 779 - RNA, 21 nt, Homo sapiens
aaagguacaucuucagagucc

SEQ ID NO: 780 - RNA, 21 nt, Homo sapiens
aauagguacaucuucagaguc

SEQ ID NO: 781 - RNA, 21 nt, Homo sapiens
aaaccauagguacaucuucag

SEQ ID NO: 782 - RNA, 21 nt, Homo sapiens
aauuccuacuaggaccauagg

SEQ ID NO: 783 - RNA, 21 nt, Homo sapiens
aauuuccuacuaggaccauag

SEQ ID NO: 784 - RNA, 21 nt, Homo sapiens
aaauuuauuuccuacuaggac

SEQ ID NO: 785 - RNA, 21 nt, Homo sapiens
aaaaucacauuuauuuccuac

SEQ ID NO: 786 - RNA, 21 nt, Homo sapiens
aaugucuacuguucuagaagg

SEQ ID NO: 787 - RNA, 21 nt, Homo sapiens
aauuugugucuacuguucuag

SEQ ID NO: 788 - RNA, 21 nt, Homo sapiens
aauguuuugugucuacuguuc

SEQ ID NO: 789 - RNA, 21 nt, Homo sapiens
aaaaguccugagccuguuuug

SEQ ID NO: 790 - RNA, 21 nt, Homo sapiens
aauugcuaaguccugagccug

SEQ ID NO: 791 - RNA, 21 nt, Homo sapiens
aaucuugcuaaguccugagcc

SEQ ID NO: 792 - RNA, 21 nt, Homo sapiens
aaaacuucuugcuaaguccug

SEQ ID NO: 793 - RNA, 21 nt, Homo sapiens
aauccauaacuucuugcuaag

SEQ ID NO: 794 - RNA, 21 nt, Homo sapiens
aagaauuccauaacuucuugc

SEQ ID NO: 795 - RNA, 21 nt, Homo sapiens
aaaaaggaauuccauaacuuc

SEQ ID NO: 796 - RNA, 21 nt, Homo sapiens
aaauaaaaggaauuccauaac

SEQ ID NO: 797 - RNA, 21 nt, Homo sapiens
aauuucaauaaaaggaauucc

SEQ ID NO: 798 - RNA, 21 nt, Homo sapiens
aauugucuuugcugauguuuc

SEQ ID NO: 799 - RNA, 21 nt, Homo sapiens
aaugucuugucuuugcugaug

SEQ ID NO: 800 - RNA, 21 nt, Homo sapiens
aacacccugucuugucuuugc

SEQ ID NO: 801 - RNA, 21 nt, Homo sapiens
aaacacccugucuugucuuug

SEQ ID NO: 802 - RNA, 21 nt, Homo sapiens
aaaucaacacccugucuuguc

SEQ ID NO: 803 - RNA, 21 nt, Homo sapiens
aaucaucaacacccugucuug

SEQ ID NO: 804 - RNA, 21 nt, Homo sapiens
aaaggcaucaucaacacccug

SEQ ID NO: 805 - RNA, 21 nt, Homo sapiens
aaagaaggcaucaucaacacc

SEQ ID NO: 806 - RNA, 21 nt, Homo sapiens
aauagaaggcaucaucaacac

SEQ ID NO: 807 - RNA, 21 nt, Homo sapiens
aauauagaaggcaucaucaac

SEQ ID NO: 808 - RNA, 21 nt, Homo sapiens
aaaauguauagaaggcaucau

SEQ ID NO: 809 - RNA, 21 nt, Homo sapiens
aacuaauguauagaaggcauc

SEQ ID NO: 810 - RNA, 21 nt, Homo sapiens
aagaacuaauguauagaaggc

SEQ ID NO: 811 - RNA, 21 nt, Homo sapiens
aaucgaacuaauguauagaag

SEQ ID NO: 812 - RNA, 21 nt, Homo sapiens
aauucucgaacuaauguauag

SEQ ID NO: 813 - RNA, 21 nt, Homo sapiens
aauauguuuucgaauuucucg

SEQ ID NO: 814 - RNA, 21 nt, Homo sapiens
aauuauguuuucgaauuucuc

SEQ ID NO: 815 - RNA, 21 nt, Homo sapiens
aaucuuuucuuuauguuuucg

SEQ ID NO: 816 - RNA, 21 nt, Homo sapiens
aaaucuuuucuuuauguuuuc

SEQ ID NO: 817 - RNA, 21 nt, Homo sapiens
aaugcucaucuuuucuuuaug

SEQ ID NO: 818 - RNA, 21 nt, Homo sapiens
aauucuuuuuaccaucuuugc

SEQ ID NO: 819 - RNA, 21 nt, Homo sapiens
aacuucuuuuuaccaucuuug

SEQ ID NO: 820 - RNA, 21 nt, Homo sapiens
aauuuucuucuuuuuaccauc

SEQ ID NO: 821 - RNA, 21 nt, Homo sapiens
aaucuuuuucuucuuuuuacc

SEQ ID NO: 822 - RNA, 21 nt, Homo sapiens
aauucuuuuucuucuuuuuac

SEQ ID NO: 823 - RNA, 21 nt, Homo sapiens
aacuuugacuucuuuuucuuc

SEQ ID NO: 824 - RNA, 21 nt, Homo sapiens
aaugucuuugacuucuuuuuc

SEQ ID NO: 825 - RNA, 21 nt, Homo sapiens
aaacacuuugucuuugacuuc

SEQ ID NO: 826 - RNA, 21 nt, Homo sapiens
aauacacacuuugucuuugac

SEQ ID NO: 827 - RNA, 21 nt, Homo sapiens
aaauuacacacuuugucuuug

SEQ ID NO: 828 - RNA, 21 nt, Homo sapiens
aacauaauuacacacuuuguc

SEQ ID NO: 829 - RNA, 21 nt, Homo sapiens
aauacauaauuacacacuuug

SEQ ID NO: 830 - RNA, 21 nt, Homo sapiens
aaaagaaaaaaguacaaauug

SEQ ID NO: 831 - RNA, 21 nt, Homo sapiens
aaugccuuaagaaaaaaguac

SEQ ID NO: 832 - RNA, 21 nt, Homo sapiens
aaguaugccuuaagaaaaaag

SEQ ID NO: 833 - RNA, 21 nt, Homo sapiens
aauguacuaguaugccuuaag

SEQ ID NO: 834 - RNA, 21 nt, Homo sapiens
aauaccacuuguacuaguaug

SEQ ID NO: 835 - RNA, 21 nt, Homo sapiens
aaaaauuaccacuuguacuag

SEQ ID NO: 836 - RNA, 21 nt, Homo sapiens
aacaaaaauuaccacuuguac

SEQ ID NO: 837 - RNA, 21 nt, Homo sapiens
aaaauguacaaaaauuaccac

SEQ ID NO: 838 - RNA, 21 nt, Homo sapiens
aauguaauguacaaaaauuac

SEQ ID NO: 839 - RNA, 21 nt, Homo sapiens
aaauaauuuaguguaauguac

SEQ ID NO: 840 - RNA, 21 nt, Homo sapiens
aacuaauaauuuaguguaaug

SEQ ID NO: 841 - RNA, 21 nt, Homo sapiens
aaacaaaugcuaauaauuuag

SEQ ID NO: 842 - RNA, 21 nt, Homo sapiens
aaguaaugcuaaaacaaaugc

SEQ ID NO: 843 - RNA, 21 nt, Homo sapiens
aaaauuagguaaugcuaaaac

SEQ ID NO: 844 - RNA, 21 nt, Homo sapiens
aaacagucugcauggagcagg

SEQ ID NO: 845 - RNA, 21 nt, Homo sapiens
aaaacagucugcauggagcag

SEQ ID NO: 846 - RNA, 21 nt, Homo sapiens
aacuaacagucugcauggagc

SEQ ID NO: 847 - RNA, 21 nt, Homo sapiens
aaaagcuaacagucugcaugg

SEQ ID NO: 848 - RNA, 22 nt, Homo sapiens
aauaagguaaaagcuaacaguc

SEQ ID NO: 849 - RNA, 21 nt, Homo sapiens
aaauuuaagguaaaagcuaac

SEQ ID NO: 850 - RNA, 21 nt, Homo sapiens
aaaagcauuuaagguaaaagc

SEQ ID NO: 851 - RNA, 21 nt, Homo sapiens
aauaagcauuuaagguaaaag

SEQ ID NO: 852 - RNA, 21 nt, Homo sapiens
aaacuuagaggaaaaaaaaac

SEQ ID NO: 853 - RNA, 21 nt, Homo sapiens
aaaaaacucugggaauacugg

SEQ ID NO: 854 - RNA, 21 nt, Homo sapiens
aauucaaaaaccaaaacucug

SEQ ID NO: 855 - RNA, 21 nt, Homo sapiens
aaaguucaaaaaccaaaacuc

SEQ ID NO: 856 - RNA, 21 nt, Homo sapiens
aauuuucacaggcauugcuag

SEQ ID NO: 857 - RNA, 21 nt, Homo sapiens
aauucaguuucuuuuucacag

SEQ ID NO: 858 - RNA, 21 nt, Homo sapiens
aauauucaguuucuuuuucac

SEQ ID NO: 859 - RNA, 21 nt, Homo sapiens
aaagaaaucuuagguauucag

SEQ ID NO: 860 - RNA, 21 nt, Homo sapiens
aaacagaaaucuuagguauuc

SEQ ID NO: 861 - RNA, 21 nt, Homo sapiens
aaaauaagaaguaaucaacug

SEQ ID NO: 862 - RNA, 21 nt, Homo sapiens
aaaaaauaagaaguaaucaac

SEQ ID NO: 863 - RNA, 21 nt, Homo sapiens
aaagaaaaauaagaaguaauc

SEQ ID NO: 864 - RNA, 21 nt, Homo sapiens
aaaauugguaagaaaaauaag

SEQ ID NO: 865 - RNA, 21 nt, Homo sapiens
aaaacauucacaauugguaag

SEQ ID NO: 866 - RNA, 21 nt, Homo sapiens
aauuguuucacaccaacauuc

SEQ ID NO: 867 - RNA, 21 nt, Homo sapiens
aaucauuaauuuguuucacac

SEQ ID NO: 868 - RNA, 21 nt, Homo sapiens
aaagauaaaacacagaauagg

SEQ ID NO: 869 - RNA, 21 nt, Homo sapiens
aauagauaaaacacagaauag

SEQ ID NO: 870 - RNA, 21 nt, Homo sapiens
aaaguaauuaauccauuuaug

SEQ ID NO: 871 - RNA, 21 nt, Homo sapiens
aaaguaaaaaccaauuagaag

SEQ ID NO: 872 - RNA, 21 nt, Homo sapiens
aauucaguaaaaaccaauuag

SEQ ID NO: 873 - RNA, 21 nt, Homo sapiens
aaucaauguuucaguaaaaac

SEQ ID NO: 874 - RNA, 21 nt, Homo sapiens
aaaaacuauaggacaugaugc

SEQ ID NO: 875 - RNA, 21 nt, Homo sapiens
aaagacaaaaccuuugugaac

SEQ ID NO: 876 - RNA, 21 nt, Homo sapiens
aaaaugugaaaaggaaauggc

SEQ ID NO: 877 - RNA, 21 nt, Homo sapiens
aauaaugugaaaaggaaaugg

SEQ ID NO: 878 - RNA, 21 nt, Homo sapiens
aagcuaacagucugcauggag

SEQ ID NO: 879 - RNA, 21 nt, Homo sapiens
aaaaagcuaacagucugcaug

SEQ ID NO: 880 - RNA, 21 nt, Homo sapiens
aaguaaaagcuaacagucugc

SEQ ID NO: 881 - RNA, 21 nt, Homo sapiens
aauuaagguaaaagcuaacag

SEQ ID NO: 882 - RNA, 21 nt, Homo sapiens
aauuaaaauaagcauuuaagg

SEQ ID NO: 883 - RNA, 21 nt, Homo sapiens
aaaaaaaaaacuuccacuguc

SEQ ID NO: 884 - RNA, 21 nt, Homo sapiens
aaaaaaaaaaacuuccacugu

SEQ ID NO: 885 - RNA, 21 nt, Homo sapiens
aagaauacuggcacuuagagg

SEQ ID NO: 886 - RNA, 21 nt, Homo sapiens
aaugggaauacuggcacuuag

SEQ ID NO: 887 - RNA, 21 nt, Homo sapiens
aaacucugggaauacuggcac

SEQ ID NO: 888 - RNA, 21 nt, Homo sapiens
aaaaacucugggaauacuggc

SEQ ID NO: 889 - RNA, 21 nt, Homo sapiens
aaaaaacucugggaauacugg

SEQ ID NO: 890 - RNA, 21 nt, Homo sapiens
aacaaaacucugggaauacug

SEQ ID NO: 891 - RNA, 21 nt, Homo sapiens
aaaccaaaacucugggaauac

SEQ ID NO: 892 - RNA, 21 nt, Homo sapiens
aacaaaaaccaaaacucuggg

SEQ ID NO: 893 - RNA, 21 nt, Homo sapiens
aaucaaaaaccaaaacucugg

SEQ ID NO: 894 - RNA, 21 nt, Homo sapiens
aaguucaaaaaccaaaacucu

SEQ ID NO: 895 - RNA, 21 nt, Homo sapiens
aacuaguucaaaaaccaaaac

SEQ ID NO: 896 - RNA, 21 nt, Homo sapiens
aacauugcuaguucaaaaacc

SEQ ID NO: 897 - RNA, 21 nt, Homo sapiens
aaucacaggcauugcuaguuc

SEQ ID NO: 898 - RNA, 21 nt, Homo sapiens
aaucuuuuucacaggcauugc

SEQ ID NO: 899 - RNA, 21 nt, Homo sapiens
aauucuuuuucacaggcauug

SEQ ID NO: 900 - RNA, 21 nt, Homo sapiens
aacaguuucuuuuucacaggc

SEQ ID NO: 901 - RNA, 21 nt, Homo sapiens
aaucaguuucuuuuucacagg

SEQ ID NO: 902 - RNA, 21 nt, Homo sapiens
aaguauucaguuucuuuuuca

SEQ ID NO: 903 - RNA, 21 nt, Homo sapiens
aaaucuuagguauucaguuuc

SEQ ID NO: 904 - RNA, 21 nt, Homo sapiens
aaaccaaaaaccccaagacag

SEQ ID NO: 905 - RNA, 21 nt, Homo sapiens
aagcaccaaaaaccccaagac

SEQ ID NO: 906 - RNA, 21 nt, Homo sapiens
aaaugcaccaaaaaccccaag

SEQ ID NO: 907 - RNA, 21 nt, Homo sapiens
aacaccaaaaacacugcaugc

SEQ ID NO: 908 - RNA, 21 nt, Homo sapiens
aaaacugcaugcaccaaaaac

SEQ ID NO: 909 - RNA, 21 nt, Homo sapiens
aauuuaucuaaugugaaaagg

SEQ ID NO: 910 - RNA, 21 nt, Homo sapiens
aaauuuaucuaaugugaaaag

SEQ ID NO: 911 - RNA, 21 nt, Homo sapiens
aaaauaauccccauuucauac

SEQ ID NO: 912 - RNA, 21 nt, Homo sapiens
aaaaauauagccccaaaaugg

SEQ ID NO: 913 - RNA, 21 nt, Homo sapiens
aauaaauauagccccaaaaug

SEQ ID NO: 914 - RNA, 21 nt, Homo sapiens
aauacuuguuaaaaucuuuuc

SEQ ID NO: 915 - RNA, 21 nt, Homo sapiens
aaugagaauuuuuuauacuug

SEQ ID NO: 916 - RNA, 21 nt, Homo sapiens
aaacuacauuuaauuccuaug

SEQ ID NO: 917 - RNA, 21 nt, Homo sapiens
aaaguuauacuaugaaagagc

SEQ ID NO: 918 - RNA, 21 nt, Homo sapiens
aaaaguuauacuaugaaagag

SEQ ID NO: 919 - RNA, 21 nt, Homo sapiens
aauaaaguuauacuaugaaag

SEQ ID NO: 920 - RNA, 21 nt, Homo sapiens
aauugaagaaaagauuuaaag

SEQ ID NO: 921 - RNA, 21 nt, Homo sapiens
aaucaaagacucaaguugaag

SEQ ID NO: 922 - RNA, 21 nt, Homo sapiens
aaucuucaaagacucaaguug

SEQ ID NO: 923 - RNA, 21 nt, Homo sapiens
aaaaacuaucuucaaagacuc

SEQ ID NO: 924 - RNA, 21 nt, Homo sapiens
aauaaaacuaucuucaaagac

SEQ ID NO: 925 - RNA, 21 nt, Homo sapiens
aaauuaaaacuaucuucaaag

SEQ ID NO: 926 - RNA, 21 nt, Homo sapiens
aaaccuaauaagcuauaacug

SEQ ID NO: 927 - RNA, 21 nt, Homo sapiens
aaucuucaacaccuaauaagc

SEQ ID NO: 928 - RNA, 21 nt, Homo sapiens
aaugaaacucucuaugaaagc

SEQ ID NO: 929 - RNA, 21 nt, Homo sapiens
aauuugacuaaccaaugcaug

SEQ ID NO: 930 - RNA, 21 nt, Homo sapiens
aauuaagaaacaaaagcaaug

SEQ ID NO: 931 - RNA, 21 nt, Homo sapiens
aauuucuuaagaaacaaaagc

SEQ ID NO: 932 - RNA, 21 nt, Homo sapiens
aauuuucuuaagaaacaaaag

SEQ ID NO: 933 - RNA, 21 nt, Homo sapiens
aaaagaguuuguuuucuuaag

SEQ ID NO: 934 - RNA, 21 nt, Homo sapiens
aauaaaaaagaguuuguuuuc

SEQ ID NO: 935 - RNA, 21 nt, Homo sapiens
aaaaacuuuuucacuucauug

SEQ ID NO: 936 - RNA, 21 nt, Homo sapiens
aauguaaaacuuuuucacuuc

SEQ ID NO: 937 - RNA, 21 nt, Homo sapiens
aaauuuaaccaguguuaagag

SEQ ID NO: 938 - RNA, 21 nt, Homo sapiens
aauaauuuaaccaguguuaag

SEQ ID NO: 939 - RNA, 21 nt, Homo sapiens
aauaugcaauguuaauuuaac

SEQ ID NO: 940 - RNA, 21 nt, Homo sapiens
aaacuugaaaaguguuuaugc

SEQ ID NO: 941 - RNA, 21 nt, Homo sapiens
aaaaauuuaucaaaaggauug

SEQ ID NO: 942 - RNA, 21 nt, Homo sapiens
aauaucuagaaccuaagucac

SEQ ID NO: 943 - RNA, 21 nt, Homo sapiens
aaaaaaucagaguccuaaaag

SEQ ID NO: 944 - RNA, 21 nt, Homo sapiens
aaaaauggauaguaagugaug

SEQ ID NO: 945 - RNA, 21 nt, Homo sapiens
aaaagaaauggauaguaagug

SEQ ID NO: 946 - RNA, 21 nt, Homo sapiens
aaugaagaaauggauaguaag

SEQ ID NO: 947 - RNA, 21 nt, Homo sapiens
aaaacaugaagaaauggauag

SEQ ID NO: 948 - RNA, 21 nt, Homo sapiens
aauuuuaacaugaagaaaugg

SEQ ID NO: 949 - RNA, 21 nt, Homo sapiens
aaaagaguuugagaugacuuc

SEQ ID NO: 950 - RNA, 21 nt, Homo sapiens
aaaaacuaagaguuugagaug

SEQ ID NO: 951 - RNA, 21 nt, Homo sapiens
aaaaaaaacuaagaguuugag

SEQ ID NO: 952 - RNA, 21 nt, Homo sapiens
aauggaauauaaauuacauag

SEQ ID NO: 953 - RNA, 21 nt, Homo sapiens
aaaaauaaguguauccuuaug

SEQ ID NO: 954 - RNA, 21 nt, Homo sapiens
aaaaaaauuuacagauugugc

SEQ ID NO: 955 - RNA, 21 nt, Homo sapiens
aaugaagaugguguaacauag

SEQ ID NO: 956 - DNA, 19 bp, Aequorea victoria
ggttatgtacaggaacgca